SEQUENCE LISTING (102 sequences)

SEQ ID NO: 1 - DNA, 54 bp, synthetic construct
  source 1..54: mol_type = other DNA
atggatttccaggtccagattttctccttcctgctgatttccgcaagcgtcatt

SEQ ID NO: 2 - protein, 18 aa, synthetic construct
MDFQVQIFSFLLISASVI

SEQ ID NO: 3 - DNA, 30 bp, synthetic construct
  source 1..30: mol_type = other DNA
gaacagaaactgattagcgaagaagacctg

SEQ ID NO: 4 - protein, 10 aa, synthetic construct
EQKLISEEDL

SEQ ID NO: 5 - DNA, 27 bp, synthetic construct
  source 1..27: mol_type = other DNA
actagtggcggaggaggatcactcgag

SEQ ID NO: 6 - protein, 9 aa, synthetic construct
TSGGGGSLE

SEQ ID NO: 7 - DNA, 75 bp, synthetic construct
  source 1..75: mol_type = other DNA
aaccccgggggaggaggagggagcgggggaggaggcagcggcgggggaggctctggaggaggagggagcggatcc

SEQ ID NO: 8 - protein, 25 aa, synthetic construct
NPGGGGGSGGGGSGGGGSGGGGSGS

SEQ ID NO: 9 - DNA, 66 bp, synthetic construct
  source 1..66: mol_type = other DNA
agcggacaggtgctgctggaatccaatatcaaagtcctgcccacttggtctacccccgtgcagcct

SEQ ID NO: 10 - protein, 22 aa, synthetic construct
SGQVLLESNIKVLPTWSTPVQP

SEQ ID NO: 11 - DNA, 51 bp, synthetic construct
  source 1..51: mol_type = other DNA
gccgaagcagcagcaaaggaggccgcagcgaaggaagcagctgcgaaggcc

SEQ ID NO: 12 - protein, 17 aa, synthetic construct
AEAAAKEAAAKEAAAKA

SEQ ID NO: 13 - DNA, 81 bp, synthetic construct
  source 1..81: mol_type = other DNA
gccgaggcagctgcaaaggaagctgcggcgaaggaggccgcagcgaaagaagcagcggcaaaagaagcagccgccaaagcc

SEQ ID NO: 14 - protein, 27 aa, synthetic construct
AEAAAKEAAAKEAAAKEAAAKEAAAKA

SEQ ID NO: 15 - DNA, 576 bp, synthetic construct
  source 1..576: mol_type = other DNA
atcgtagtgttggcatttcaaaaagcgtctagcatcgtctataagaaggaaggtgaacaagtcgagttttctttcccccttgcatttacggtggaaaagcttacgggtagcggcgagctgtggtggcaagctgaacgggcttcaagctcaaaatcttggattacttttgacttgaagaacaaagaggtgagtgtcaaaagagttactcaggacccaaagcttcaaatggggaagaaacttccgctgcacctgacgttgcctcaggccctgcctcaatatgccggctcaggcaatctgaccctcgcgctggaagctaagaccggaaaattgcaccaggaagtcaatttggttgtgatgcgcgccactcagctccaaaaaaatctcacttgcgaggtatgggggcctacgagcccaaaacttatgctgtctttgaagcttgaaaacaaggaagcgaaagtttctaagcgcgagaaagcggtatgggttttgaatcctgaggctggaatgtggcaatgcctcctgagcgatagcgggcaggtgctgttggagagcaacatcaaggttttgccagcagcc

SEQ ID NO: 16 - protein, 192 aa, synthetic construct
IVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPAA

SEQ ID NO: 17 - DNA, 60 bp, synthetic construct
  source 1..60: mol_type = other DNA
atggagacccccgcccagctgctgttcctgctgctgctgtggctgcccgacaccaccggc

SEQ ID NO: 18 - protein, 20 aa, synthetic construct
METPAQLLFLLLLWLPDTTG

SEQ ID NO: 19 - DNA, 63 bp, synthetic construct
  source 1..63: mol_type = other DNA
atggccctgccagtgaccgccctgctgctgccactggccctgctgctgcacgccgccagaccc

SEQ ID NO: 20 - protein, 21 aa, synthetic construct
MALPVTALLLPLALLLHAARP

SEQ ID NO: 21 - DNA, 54 bp, synthetic construct
  source 1..54: mol_type = other DNA
ggatctaccagcggatccggcaagcctggcagcggagagggatccacaaaggga

SEQ ID NO: 22 - protein, 18 aa, synthetic construct
GSTSGSGKPGSGEGSTKG

SEQ ID NO: 23 - DNA, 45 bp, synthetic construct
  source 1..45: mol_type = other DNA
ggcggcggcggaagtggaggaggaggctcaggcggaggagggagc

SEQ ID NO: 24 - protein, 15 aa, synthetic construct
GGGGSGGGGSGGGGS

SEQ ID NO: 25 - DNA, 60 bp, synthetic construct
  source 1..60: mol_type = other DNA
ggaggaggagggagcgggggaggaggcagcggcgggggaggctctggaggaggagggagc

SEQ ID NO: 26 - protein, 20 aa, synthetic construct
GGGGSGGGGSGGGGSGGGGS

SEQ ID NO: 27 - DNA, 15 bp, synthetic construct
  source 1..15: mol_type = other DNA
ggaggaggagggagc

SEQ ID NO: 28 - protein, 5 aa, synthetic construct
GGGGS

SEQ ID NO: 29 - DNA, 63 bp, synthetic construct
  source 1..63: mol_type = other DNA
atggccctgccagtgaccgccctgctgctgccactggccctgctgctgcacgccgcccggcct

SEQ ID NO: 30 - protein, 21 aa, synthetic construct
MALPVTALLLPLALLLHAARP

SEQ ID NO: 31 - DNA, 750 bp, synthetic construct
  source 1..750: mol_type = other DNA
atggacatccagatgactcagaccacaagctccctgtctgcaagtctgggcgaccgggtgacaatctcctgcagagcctctcaggatattaggaactacctgaattggtatcagcagaaacctgatggcacagtcaagctgctgatctactataccagccggctgcactcaggcgtgccaagcaaattctcaggaagcggctccgggactgactactccctgaccatctctaacctggagcaggaagatattgctacctatttctgccagcagggcaatacactgccctggacttttgccggaggcaccaaactggagatcaaggggggaggcgggagtggaggcgggggatcaggaggaggaggcagcggaggaggagggtccgaggtccagctgcagcagagcggaccagaactggtgaagcccggagcaagtatgaaaatctcctgtaaggcctcaggatacagcttcaccggctatacaatgaactgggtgaaacagtcccatggcaagaacctggaatggatggggctgattaatccttacaaaggcgtcagcacctataatcagaagtttaaagacaaggccacactgactgtggataagtctagttcaaccgcttacatggagctgctgtccctgacatctgaagacagtgccgtgtactattgtgctcggtctggctactatggggacagtgattggtacttcgatgtctggggacagggcactaccctgaccgtgttttct

SEQ ID NO: 32 - protein, 250 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 33 - DNA, 769 bp, synthetic construct
  source 1..769: mol_type = other DNA
atggccgacatcgtgctgacacagagccccgccatcatgtctgccagccctggcgagaaagtgaccatgacctgtagcgccagcagcagcgtgtcctacatgaactggtatcagcagaagtccggcaccagccccaagcggtggatctacgacacaagcaagctggcctctggcgtgcccgcccactttagaggctctggcagcggcacaagctacagcctgaccatcagcggcatggaagccgaggatgccgccacctactactgccagcagtggtccagcaaccccttcacctttggctccggcacaaagctggaaatcaaccgggccgacaccgcccctacaggcggcggaggatctggcggaggcggatctgggggcggaggaagtggggggggaggatctatggctcaggtgcagctgcagcagtctggcgccgaactggctagacctggcgcctccgtgaagatgagctgcaaggccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgtcctccctgaccagcgaggacagcgccgtgtactactgcgcccggtactacgacgaccactactccctggactactggggccagggcaccacactgaccgtgtctagta

SEQ ID NO: 34 - protein, 256 aa, synthetic construct
MADIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTGGGGSGGGGSGGGGSGGGGSMAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 35 - DNA, 747 bp, synthetic construct
  source 1..747: mol_type = other DNA
cagaccgtggtgacccaggagcccagcctgaccgtgagccccggcggcaccgtgaccctgacctgcggcagcagcaccggcgccgtgaccagcggctactaccccaactgggtgcagcagaagcccggccaggcccccaggggcctgatcggcggcaccaagttcctggcccccggcacccccgccaggttcagcggcagcctgctgggcggcaaggccgccctgaccctgagcggcgtgcagcccgaggacgaggccgagtactactgcgccctgtggtacagcaacaggtgggtgttcggcggcggcaccaagctgaccgtgctgggcggcggcggcagcggcggcggcggcagcggcggcggcggcagcgaggtgcagctgctggagagcggcggcggcctggtgcagcccggcggcagcctgaagctgagctgcgccgccagcggcttcaccttcaacatctacgccatgaactgggtgaggcaggcccccggcaagggcctggagtgggtggccaggatcaggagcaagtacaacaactacgccacctactacgccgacagcgtgaagagcaggttcaccatcagcagggacgacagcaagaacaccgcctacctgcagatgaacaacctgaagaccgaggacaccgccgtgtactactgcgtgaggcacggcaacttcggcaacagctacgtgagcttcttcgcctactggggccagggcaccctggtgaccgtgagcagc

SEQ ID NO: 36 - protein, 249 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGSGGGGSGGGGSEVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 37 - DNA, 720 bp, synthetic construct
  source 1..720: mol_type = other DNA
gacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagggcggcggcggcagcggcggcggcggcagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagc

SEQ ID NO: 38 - protein, 240 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 39 - DNA, 735 bp, synthetic construct
  source 1..735: mol_type = other DNA
atggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcg

SEQ ID NO: 40 - protein, 245 aa, synthetic construct
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 41 - DNA, 735 bp, synthetic construct
  source 1..735: mol_type = other DNA
atggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcg

SEQ ID NO: 42 - protein, 245 aa, synthetic construct
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSS

SEQ ID NO: 43 - DNA, 750 bp, synthetic construct
  source 1..750: mol_type = other DNA
atggacatccagatgactcagaccacaagctccctgtctgcaagtctgggcgaccgggtgacaatctcctgcagagcctctcaggatattaggaactacctgaattggtatcagcagaaacctgatggcacagtcaagctgctgatctactataccagccggctgcactcaggcgtgccaagcaaattctcaggaagcggctccgggactgactactccctgaccatctctaacctggagcaggaagatattgctacctatttctgccagcagggcaatacactgccctggacttttgccggaggcaccaaactggagatcaaggggggaggcgggagtggaggcgggggatcaggaggaggaggcagcggaggaggagggtccgaggtccagctgcagcagagcggaccagaactggtgaagcccggagcaagtatgaaaatctcctgtaaggcctcaggatacagcttcaccggctatacaatgaactgggtgaaacagtcccatggcaagaacctggaatggatggggctgattaatcctaccaaaggcgtcagcacctataatcagaagtttaaagacaaggccacactgactgtggataagtctagttcaaccgcttacatggagctgctgtccctgacatctgaagacagtgccgtgtactattgtgctcggtctggctactatggggacagtgattggtacttcgatgtctggggacagggcactaccctgaccgtgttttct

SEQ ID NO: 44 - protein, 250 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPTKGVSTYNQKFKDKATLTVDKSSSTAYMELLSLTSEDSAVYYCARSGYYGDSDWYFDVWGQGTTLTVFS

SEQ ID NO: 45 - DNA, 252 bp, synthetic construct
  source 1..252: mol_type = other DNA
agcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 46 - protein, 84 aa, synthetic construct
SGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 47 - DNA, 237 bp, synthetic construct
  source 1..237: mol_type = other DNA
ctcgagctgaggcccgaggcttctagacctgctgccggcggagccgtgcacaccagaggcctggacttcgccagcgacatctacatctgggcccctctggccggcacctgtggcgtgctgctgctgagcctggtcatcaccctgtactgcaaccaccggaaccggcggagagtgtgcaagtgccccagacccgtggtcaagagcggcgacaagcccagcctgagcgccagatacgtg

SEQ ID NO: 48 - protein, 79 aa, synthetic construct
LELRPEASRPAAGGAVHTRGLDFASDIYIWAPLAGTCGVLLLSLVITLYCNHRNRRRVCKCPRPVVKSGDKPSLSARYV

SEQ ID NO: 49 - DNA, 234 bp, synthetic construct
  source 1..234: mol_type = other DNA
ctcgagctgaggcccgaggcttctagacctgctgccggcggagccgtgcacaccagaggcctggacttcgccagcgacatctacatctgggcccctctggccggcacctgtggcgtgctgctgctgagcctggtcatcaccctgtacctgtgctgcagacggcggagagtgtgcaagtgccccagacccgtggtcaagagcggcgacaagcccagcctgagcgccagatacgtg

SEQ ID NO: 50 - protein, 78 aa, synthetic construct
LELRPEASRPAAGGAVHTRGLDFASDIYIWAPLAGTCGVLLLSLVITLYLCCRRRRVCKCPRPVVKSGDKPSLSARYV

SEQ ID NO: 51 - DNA, 213 bp, synthetic construct
  source 1..213: mol_type = other DNA
ctcgagaagaagtccaccctgaagaaacgggtgtcccggctgcccagacccgagacacagaagggccccctgagcagccctatcaccctgggactgctggtggccggcgtgctggtgctgctggtgtctctgggagtggccatccacctgtgctgccggcggagaagggcctgcaagtgccccagactgcggttcatgaagcagttctacaag

SEQ ID NO: 52 - protein, 71 aa, synthetic construct
LEKKSTLKKRVSRLPRPETQKGPLSSPITLGLLVAGVLVLLVSLGVAIHLCCRRRRACKCPRLRFMKQFYK

SEQ ID NO: 53 - DNA, 1926 bp, synthetic construct
  source 1..1926: mol_type = other DNA
atggctctgcctgtgacagctctgttgctgcctctggctctgctgctgcatgctgctagacctgaggtgcagctggtgggcagcggcggcggcctggtgcagcccgagggcagcctgaagctgagctgcgccgccagcggcttcaccttcaagaccaacgccatgaactgggtgaggcaggcccccggcaagggcctggagtgggtggccaggatcaggaacaagaccaacaactacgccacctactacgccgacagcgtgaaggacaggttcaccatcagcagggacgacagccagagcatgctgtacctgcagatgaacaacctgaagaccgaggacaccgccatgtacttctgcgtggccggcaacagcttcgcctactggggccagggcaccctggtgaccgtgagcgccgggtcaacctctggtagcggtaagcctggctccggcgaaggctccacaaagggtgacatcgtgatgagccagagccccagcagcctggccgtgagcgtgggcgagaaggtgaccgtgaactgcaagagcagccagagcctgctgtacagcaacaaccagaagaactacctggcctggtaccagcagaagcccggccagagccccaagctgctgatctactgggccagcaccagggagtacggcgtgcccgacaggttcaccggcagcggcagcggcaccgacttcaccctgaccatcagcagcgtgaaggccgaggacctggccgtgtactactgccagcagtactacaactaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 54 - protein, 642 aa, synthetic construct
MALPVTALLLPLALLLHAARPEVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSAGSTSGSGKPGSGEGSTKGDIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 55 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagaaggtgaccgtgaactgcaagagcagccagagcctgctgtacagcaacaaccagaagaactacctggcctggtaccagcagaagcccggccagagccccaagctgctgatctactgggccagcaccagggagtacggcgtgcccgacaggttcaccggcagcggcagcggcaccgacttcaccctgaccatcagcagcgtgaaggccgaggacctggccgtgtactactgccagcagtactacaactaccccctgaccttcggcgccggcaccaagctggagctgaaggggtcaacctctggtagcggtaagcctggctccggcgaaggctccacaaagggtgaggtgcagctggtgggcagcggcggcggcctggtgcagcccgagggcagcctgaagctgagctgcgccgccagcggcttcaccttcaagaccaacgccatgaactgggtgaggcaggcccccggcaagggcctggagtgggtggccaggatcaggaacaagaccaacaactacgccacctactacgccgacagcgtgaaggacaggttcaccatcagcagggacgacagccagagcatgctgtacctgcagatgaacaacctgaagaccgaggacaccgccatgtacttctgcgtggccggcaacagcttcgcctactggggccagggcaccctggtgaccgtgagcgccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 56 - protein, 642 aa, synthetic construct
MALPVTALLLPLALLLHAARPDIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELKGSTSGSGKPGSGEGSTKGEVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSAEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 57 - DNA, 1926 bp, synthetic construct
  source 1..1926: mol_type = other DNA
atggctctgcctgtgacagctctgttgctgcctctggctctgctgctgcatgctgctagacctgaggtgcagctggtggagaccggcggcggcctggtgcagcccaagggcagcctgaagctgagctgcgccgccagcggcttcaccttcaacaccaacgccatgaactgggtgaggcaggcccccggcaagggcctgcagtgggtggccagggtgaggaacaagaccaacaactacgccacctactacgccgacagcgtgaaggacaggttcaccatcagcagggacgacagccagagcatgctgtacctgcagatgaacaacctgaagaccgaggacaccgccatctactactgcgtgggcggcaacagcttcgcctactggggccagggcaccctggtgaccgtgagcgccgggtcaacctctggtagcggtaagcctggctccggcgaaggctccacaaagggtgacatcgtgatgagccagagccccagcagcctggccgtgagcgtgggcgagaaggtgaccatgagctgcaagagcagccagagcctgctgtacagcaacaaccagaagaactacctggcctggtaccaccagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacagctaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 58 - protein, 642 aa, synthetic construct
MALPVTALLLPLALLLHAARPEVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSAGSTSGSGKPGSGEGSTKGDIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 59 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagaaggtgaccatgagctgcaagagcagccagagcctgctgtacagcaacaaccagaagaactacctggcctggtaccaccagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacagctaccccctgaccttcggcgccggcaccaagctggagctgaaggggtcaacctctggtagcggtaagcctggctccggcgaaggctccacaaagggtgaggtgcagctggtggagaccggcggcggcctggtgcagcccaagggcagcctgaagctgagctgcgccgccagcggcttcaccttcaacaccaacgccatgaactgggtgaggcaggcccccggcaagggcctgcagtgggtggccagggtgaggaacaagaccaacaactacgccacctactacgccgacagcgtgaaggacaggttcaccatcagcagggacgacagccagagcatgctgtacctgcagatgaacaacctgaagaccgaggacaccgccatctactactgcgtgggcggcaacagcttcgcctactggggccagggcaccctggtgaccgtgagcgccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 60 - protein, 642 aa, synthetic construct
MALPVTALLLPLALLLHAARPDIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELKGSTSGSGKPGSGEGSTKGEVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSAEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 61 - DNA, 1926 bp, synthetic construct
  source 1..1926: mol_type = other DNA
atggctctgcctgtgacagctctgttgctgcctctggctctgctgctgcatgctgctagacctgaggtgcagctggtggagaccggcggcggcctggtgcagcccgagggcagcctgaagctgagctgcgccaccagcggcttcaacttcaacaccaacgccatgaactgggtgaggcaggcccccggcaagggcctggagtgggtggccagggtgaggaacaagaccaacaactacgccacctactacgccgacagcgtgaaggacaggttcaccatcagcagggacgacagccagaggatggtgttcctgcagatgaacaacctgaagaccgaggacaccgccatctactactgcgtggccggcaacagcttcgcctactggggccagggcaccctggtgaccgtgagccccgggtcaacctctggtagcggtaagcctggctccggcgaaggctccacaaagggtgacatcgtgatgagccagagccccagcagcctggccgtgagcgtgggcgagagggtgaccatgaactgcaagagcagccagagcctgctgtacagcagcaaccagaagaactacctggcctggtaccagcagaagcccggccagagccccaagctgctgatctactgggccagcaccagggagagcggcgtgcccgacaggttcaccggcagcgtgagcggcaccgacttcaccctgaccatcagcagcgtgaaggccgaggacctggccgtgtactactgccagcagtactacaactaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 62 - protein, 642 aa, synthetic construct
MALPVTALLLPLALLLHAARPEVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSPGSTSGSGKPGSGEGSTKGDIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 63 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagagggtgaccatgaactgcaagagcagccagagcctgctgtacagcagcaaccagaagaactacctggcctggtaccagcagaagcccggccagagccccaagctgctgatctactgggccagcaccagggagagcggcgtgcccgacaggttcaccggcagcgtgagcggcaccgacttcaccctgaccatcagcagcgtgaaggccgaggacctggccgtgtactactgccagcagtactacaactaccccctgaccttcggcgccggcaccaagctggagctgaaggggtcaacctctggtagcggtaagcctggctccggcgaaggctccacaaagggtgaggtgcagctggtggagaccggcggcggcctggtgcagcccgagggcagcctgaagctgagctgcgccaccagcggcttcaacttcaacaccaacgccatgaactgggtgaggcaggcccccggcaagggcctggagtgggtggccagggtgaggaacaagaccaacaactacgccacctactacgccgacagcgtgaaggacaggttcaccatcagcagggacgacagccagaggatggtgttcctgcagatgaacaacctgaagaccgaggacaccgccatctactactgcgtggccggcaacagcttcgcctactggggccagggcaccctggtgaccgtgagccccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 64 - protein, 642 aa, synthetic construct
MALPVTALLLPLALLLHAARPDIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELKGSTSGSGKPGSGEGSTKGEVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSPEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 65 - DNA, 1926 bp, synthetic construct
  source 1..1926: mol_type = other DNA
atggctctgcctgtgacagctctgttgctgcctctggctctgctgctgcatgctgctagacctgaggtgcagctggtgggcagcggcggcggcctggtgcagcccgagggcagcctgaagctgagctgcgccgccagcggcttcaccttcaagaccaacgccatgaactgggtgaggcaggcccccggcaagggcctggagtgggtggccaggatcaggaacaagaccaacaactacgccacctactacgccgacagcgtgaaggacaggttcaccatcagcagggacgacagccagagcatgctgtacctgcagatgaacaacctgaagaccgaggacaccgccatgtacttctgcgtggccggcaacagcttcgcctactggggccagggcaccctggtgaccgtgagcgccgggtcaacctctggtagcggtaagcctggctccggcgaaggctccacaaagggtgacatcgtgatgagccagagccccagcagcctggccgtgagcgtgggcgagaaggtgaccgtgaactgcaagagcagccagagcctgctgtacagcaacaaccagaagaactacctggcctggtaccagcagaagcccggccagagccccaagctgctgatctactgggccagcaccagggagtacggcgtgcccgacaggttcaccggcagcggcagcggcaccgacttcaccctgaccatcagcagcgtgaaggccgaggacctggccgtgtactactgccagcagtactacaactaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 66 - protein, 642 aa, synthetic construct
MALPVTALLLPLALLLHAARPEVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSAGSTSGSGKPGSGEGSTKGDIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 67 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagaaggtgaccgtgaactgcaagagcagccagagcctgctgtacagcaacaaccagaagaactacctggcctggtaccagcagaagcccggccagagccccaagctgctgatctactgggccagcaccagggagtacggcgtgcccgacaggttcaccggcagcggcagcggcaccgacttcaccctgaccatcagcagcgtgaaggccgaggacctggccgtgtactactgccagcagtactacaactaccccctgaccttcggcgccggcaccaagctggagctgaaggggtcaacctctggtagcggtaagcctggctccggcgaaggctccacaaagggtgaggtgcagctggtgggcagcggcggcggcctggtgcagcccgagggcagcctgaagctgagctgcgccgccagcggcttcaccttcaagaccaacgccatgaactgggtgaggcaggcccccggcaagggcctggagtgggtggccaggatcaggaacaagaccaacaactacgccacctactacgccgacagcgtgaaggacaggttcaccatcagcagggacgacagccagagcatgctgtacctgcagatgaacaacctgaagaccgaggacaccgccatgtacttctgcgtggccggcaacagcttcgcctactggggccagggcaccctggtgaccgtgagcgccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 68 - protein, 642 aa, synthetic construct
MALPVTALLLPLALLLHAARPDIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELKGSTSGSGKPGSGEGSTKGEVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSAEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 69 - DNA, 1926 bp, synthetic construct
  source 1..1926: mol_type = other DNA
atggctctgcctgtgacagctctgttgctgcctctggctctgctgctgcatgctgctagacctgaggtgcagctggtggagaccggcggcggcctggtgcagcccaagggcagcctgaagctgagctgcgccgccagcggcttcaccttcaacaccaacgccatgaactgggtgaggcaggcccccggcaagggcctgcagtgggtggccagggtgaggaacaagaccaacaactacgccacctactacgccgacagcgtgaaggacaggttcaccatcagcagggacgacagccagagcatgctgtacctgcagatgaacaacctgaagaccgaggacaccgccatctactactgcgtgggcggcaacagcttcgcctactggggccagggcaccctggtgaccgtgagcgccgggtcaacctctggtagcggtaagcctggctccggcgaaggctccacaaagggtgacatcgtgatgagccagagccccagcagcctggccgtgagcgtgggcgagaaggtgaccatgagctgcaagagcagccagagcctgctgtacagcaacaaccagaagaactacctggcctggtaccaccagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacagctaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 70 - protein, 642 aa, synthetic construct
MALPVTALLLPLALLLHAARPEVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSAGSTSGSGKPGSGEGSTKGDIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 71 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagaaggtgaccatgagctgcaagagcagccagagcctgctgtacagcaacaaccagaagaactacctggcctggtaccaccagaagcccggccagagccccaagctgctgatctactgggccagcaccagggagagcggcgtgcccgacaggttcaccggcagcggcagcggcaccgacttcaccctgaccatcagcagcgtgaaggccgaggacctggccgtgtactactgccagcagtactacagctaccccctgaccttcggcgccggcaccaagctggagctgaaggggtcaacctctggtagcggtaagcctggctccggcgaaggctccacaaagggtgaggtgcagctggtggagaccggcggcggcctggtgcagcccaagggcagcctgaagctgagctgcgccgccagcggcttcaccttcaacaccaacgccatgaactgggtgaggcaggcccccggcaagggcctgcagtgggtggccagggtgaggaacaagaccaacaactacgccacctactacgccgacagcgtgaaggacaggttcaccatcagcagggacgacagccagagcatgctgtacctgcagatgaacaacctgaagaccgaggacaccgccatctactactgcgtgggcggcaacagcttcgcctactggggccagggcaccctggtgaccgtgagcgccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 72 - protein, 642 aa, synthetic construct
MALPVTALLLPLALLLHAARPDIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELKGSTSGSGKPGSGEGSTKGEVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSAEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 73 - DNA, 1926 bp, synthetic construct
  source 1..1926: mol_type = other DNA
atggctctgcctgtgacagctctgttgctgcctctggctctgctgctgcatgctgctagacctgaggtgcagctggtggagaccggcggcggcctggtgcagcccgagggcagcctgaagctgagctgcgccaccagcggcttcaacttcaacaccaacgccatgaactgggtgaggcaggcccccggcaagggcctggagtgggtggccagggtgaggaacaagaccaacaactacgccacctactacgccgacagcgtgaaggacaggttcaccatcagcagggacgacagccagaggatggtgttcctgcagatgaacaacctgaagaccgaggacaccgccatctactactgcgtggccggcaacagcttcgcctactggggccagggcaccctggtgaccgtgagccccgggtcaacctctggtagcggtaagcctggctccggcgaaggctccacaaagggtgacatcgtgatgagccagagccccagcagcctggccgtgagcgtgggcgagagggtgaccatgaactgcaagagcagccagagcctgctgtacagcagcaaccagaagaactacctggcctggtaccagcagaagcccggccagagccccaagctgctgatctactgggccagcaccagggagagcggcgtgcccgacaggttcaccggcagcgtgagcggcaccgacttcaccctgaccatcagcagcgtgaaggccgaggacctggccgtgtactactgccagcagtactacaactaccccctgaccttcggcgccggcaccaagctggagctgaaggagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 74 - protein, 642 aa, synthetic construct
MALPVTALLLPLALLLHAARPEVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSPGSTSGSGKPGSGEGSTKGDIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 75 - DNA, 1926 bp, synthetic construct
  source 1..1926: mol_type = other DNA
atggctctgcctgtgacagctctgttgctgcctctggctctgctgctgcatgctgctagacctgacatcgtgatgagccagagccccagcagcctggccgtgagcgtgggcgagagggtgaccatgaactgcaagagcagccagagcctgctgtacagcagcaaccagaagaactacctggcctggtaccagcagaagcccggccagagccccaagctgctgatctactgggccagcaccagggagagcggcgtgcccgacaggttcaccggcagcgtgagcggcaccgacttcaccctgaccatcagcagcgtgaaggccgaggacctggccgtgtactactgccagcagtactacaactaccccctgaccttcggcgccggcaccaagctggagctgaaggggtcaacctctggtagcggtaagcctggctccggcgaaggctccacaaagggtgaggtgcagctggtggagaccggcggcggcctggtgcagcccgagggcagcctgaagctgagctgcgccaccagcggcttcaacttcaacaccaacgccatgaactgggtgaggcaggcccccggcaagggcctggagtgggtggccagggtgaggaacaagaccaacaactacgccacctactacgccgacagcgtgaaggacaggttcaccatcagcagggacgacagccagaggatggtgttcctgcagatgaacaacctgaagaccgaggacaccgccatctactactgcgtggccggcaacagcttcgcctactggggccagggcaccctggtgaccgtgagccccgagcagaaactgatcagcgaggaagatctgaatcctggcggaggcggaggaagtggtggcggaggttctggtggcggtggatcaggcggtggcggatctggatctatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgacatctggcggcggaggatctctggaatctggacaggtgctgctggaaagcaacatcaaggtgctgcccacctggtctaccccagttcagcctatggctctgattgtgcttggcggagttgccggcctgctgctctttatcggcctgggcatcttcttttgcgtgcggtgcagacatcggcggagacaggctgagagaatgagccagatcaagcggctgctgagcgagaagaaaacctgtcagtgccctcaccggttccagaaaacatgcagccccatc

SEQ ID NO: 76 - protein, 642 aa, synthetic construct
MALPVTALLLPLALLLHAARPDIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELKGSTSGSGKPGSGEGSTKGEVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSPEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 77 - protein, 5 aa, synthetic construct
TNAMN

SEQ ID NO: 78 - protein, 19 aa, synthetic construct
RIRNKTNNYATYYADSVKD

SEQ ID NO: 79 - protein, 6 aa, synthetic construct
GNSFAY

SEQ ID NO: 80 - protein, 17 aa, synthetic construct
KSSQSLLYSNNQKNYLA

SEQ ID NO: 81 - protein, 7 aa, synthetic construct
WASTREY

SEQ ID NO: 82 - protein, 9 aa, synthetic construct
QQYYNYPLT

SEQ ID NO: 83 - protein, 8 aa, synthetic construct
GFTFKTNA

SEQ ID NO: 84 - protein, 10 aa, synthetic construct
IRNKTNNYAT

SEQ ID NO: 85 - protein, 8 aa, synthetic construct
VAGNSFAY

SEQ ID NO: 86 - protein, 12 aa, synthetic construct
QSLLYSNNQKNY

SEQ ID NO: 87 - skipped

SEQ ID NO: 88 - protein, 19 aa, synthetic construct
RVRNKTNNYATYYADSVKD

SEQ ID NO: 89 - protein, 7 aa, synthetic construct
WASTRES

SEQ ID NO: 90 - protein, 9 aa, synthetic construct
QQYYSYPLT

SEQ ID NO: 91 - protein, 8 aa, synthetic construct
GFTFNTNA

SEQ ID NO: 92 - protein, 10 aa, synthetic construct
VRNKTNNYAT

SEQ ID NO: 93 - protein, 8 aa, synthetic construct
VGGNSFAY

SEQ ID NO: 94 - protein, 17 aa, synthetic construct
KSSQSLLYSSNQKNYLA

SEQ ID NO: 95 - protein, 8 aa, synthetic construct
GFNFNTNA

SEQ ID NO: 96 - protein, 12 aa, synthetic construct
QSLLYSSNQKNY

SEQ ID NO: 97 - protein, 248 aa, synthetic construct
EVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSAGSTSGSGKPGSGEGSTKGDIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELK

SEQ ID NO: 98 - protein, 248 aa, synthetic construct
DIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELKGSTSGSGKPGSGEGSTKGEVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSA

SEQ ID NO: 99 - protein, 248 aa, synthetic construct
EVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSAGSTSGSGKPGSGEGSTKGDIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELK

SEQ ID NO: 100 - protein, 248 aa, synthetic construct
DIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELKGSTSGSGKPGSGEGSTKGEVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSA

SEQ ID NO: 101 - protein, 248 aa, synthetic construct
EVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSPGSTSGSGKPGSGEGSTKGDIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELK

SEQ ID NO: 102 - protein, 248 aa, synthetic construct
DIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELKGSTSGSGKPGSGEGSTKGEVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSP

(19) United States
(12) Patent Application Publication
(10) Pub. No.: US 2025/0043021 A1
(43) Pub. Date: Feb. 6, 2025

(54) GPC3 T CELL-ANTIGEN COUPLERS AND USES THEREOF

(71) Applicant: Triumvira Immunologics USA, Inc., Austin, TX (US)

(72) Inventors: Andreas Bader, Austin, TX (US); Christopher W. Helsen, Oakville (CA); Philbert Ip, Hamilton (CA); Tania Benatar, Thornhill (CA)

(21) Appl. No.: 18/577,221

(22) PCT Filed: Jul. 7, 2022
(86) PCT No.: PCT/US2022/036393

§ 371 Date: Jan. 5, 2024

Related U.S. Application Data
(60) Provisional application No. 63/203,080, filed on Jul. 7, 2021.

(51) Int. Cl. C07K 16/30; A61K 39/00; C07K 14/725; C12N 5/0783

(52) U.S. Cl. CPC C07K 16/303; A61K 39/4632; A61K 39/4637; A61K 39/464474; C07K 14/7051; C12N 5/0636; A61K 2239/50; A61K 2239/53; A61K 2239/55; C07K 2319/03; C12N 2510/00

(57) ABSTRACT

GPC3 T cell antigen coupler (TAC) polypeptides having (i) an antigen-binding domain that binds GPC3, (ii) an antigen-binding domain that binds a protein associated with a TCR complex, and (iii) a T cell receptor signaling domain polypeptide are provided.

### CROSS-REFERENCE TO RELATED APPLICATIONS

This application claims the benefit of and priority to U.S. Provisional Patent Application No. 63/203,080, filed Jul. 7, 2021, the disclosures of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on xxxx xx, 2022, is named xxxxxxxxx.txt and is xxx,xxx bytes in size.

### SUMMARY

Disclosed herein, in certain embodiments, are polynucleotides encoding a GPC3 (Glypican 3) T cell-antigen coupler (GPC3-TAC) polypeptide.

Disclosed herein, in certain embodiments, are nucleic acids encoding a Glypican 3 (GPC3) T cell-antigen coupler (GPC3-TAC) polypeptide, the nucleic acid comprising: (a) a first polynucleotide encoding an antigen-binding domain that binds GPC3; (b) a second polynucleotide encoding an antigen-binding domain that binds a protein associated with a TCR complex; and (c) a third polynucleotide encoding a TCR co-receptor cytosolic domain and transmembrane domain; wherein components encoded by (a), components encoded by (b), and components encoded by (c) are fused directly to each other, or joined by at least one linker. In some embodiments, the first polynucleotide, the second polynucleotide, and the third polynucleotide are in order. In some embodiments, the antigen-binding domain that binds GPC3 is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds GPC3 is a designed ankyrin repeat (DARPin) polypeptide, a single chain variable fragment (scFv), or a nanobody. In some embodiments, the antigen-binding domain that binds GPC3 is a nanobody. In some embodiments, the antigen-binding domain that binds GPC3 comprises an antigen-binding domain derived from an antibody selected from codrituzumab/GC33, GPC3-C02, A1836A, 1G12, YP8, YP9, or YP9.1. In some embodiments, the antigen-binding domain that binds GPC3 comprises an amino acid sequence having at least 80%, at least 85%, at least 90%, at least 95%, at least 96%, at least 97%, at least 98%, at least 99%, or 100% sequence identity with the amino acid sequence of the antigen-binding domain(s) of codrituzumab/GC33, GPC3-C02, A1836A, 1G12, YP8, YP9, or YP9.1. In some embodiments, the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1. In some embodiments, the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1, and the non-CDR sequences of the antigen-binding domain that binds GPC3 have at least 80%, at least 85%, at least 90%, at least 95%, at least 96%, at least 97%, at least 98%, or at least 99% sequence identity with the non-CDR sequences of the amino acid sequence of codrituzumab/GC33, GPC3-C02, A1836A, 1G12, YP8, YP9, or YP9.1. In some embodiments, the antigen-binding domain comprises: (a) an HCDR1 of SEQ ID NO: 77, an HCDR2 of SEQ ID NO: 78, and an HCDR3 of SEQ ID NO: 79; and a light-chain variable domain comprising an LCDR1 of SEQ ID NO: 80, an LCDR2 of SEQ ID NO: 81, and an LCDR3 of SEQ ID NO: 82; (b) an HCDR1 of SEQ ID NO: 83, an HCDR2 of SEQ ID NO: 84, and an HCDR3 of SEQ ID NO: 85; and a light-chain variable domain comprising an LCDR1 of SEQ ID NO: 86, an LCDR2 of SEQ ID NO: 87, and an LCDR3 of SEQ ID NO: 82; (c) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 80, an LCDR2 of SEQ ID NO: 89, and an LCDR3 of SEQ ID NO: 90; (d) an HCDR1 of SEQ ID NO: 91, an HCDR2 of SEQ ID NO: 92, and an HCDR3 of SEQ ID NO: 93; and a light-chain variable domain comprising an LCDR1 of SEQ ID NO: 86, an LCDR2 of SEQ ID NO: 87, and an LCDR3 of SEQ ID NO: 90; (e) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 94, an LCDR2 of SEQ ID NO: 89, and an LCDR3 of SEQ ID NO: 82; or (f) an HCDR1 of SEQ ID NO: 95, an HCDR2 of SEQ ID NO: 92, and an HCDR3 of SEQ ID NO: 85; and a light-chain variable domain comprising an LCDR1 of SEQ ID NO: 96, an LCDR2 of SEQ ID NO: 87, and an LCDR3 of SEQ ID NO: 82. In some embodiments, the antigen-binding domain comprises an amino acid sequence having at least 80%, at least 85%, at least 90%, at least 95%, at least 96%, at least 97%, at least 98%, at least 99%, or 100% sequence identity with an amino acid sequence selected from SEQ ID NOs: 97-102. In some embodiments, the protein associated with the TCR complex is a CD3 protein. In some embodiments, the CD3 protein is a CD3γ protein, CD3δ protein and/or CD3ε protein. In some embodiments, the CD3 protein is a CD3ε protein. In some embodiments, the CD3 protein is a CD3ε protein. In some embodiments, the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds the protein associated with the TCR complex is derived from an antibody selected from UCHT1 OKT3, F6A, and L2K. In some embodiments, the antigen-binding domain that binds the protein associated with the TCR complex is a UCHT1 antigen-binding domain. In some embodiments, the UCHT1 antigen-binding domain is an scFv of UCHT1. In some embodiments, the UCHT1 antigen-binding domain comprises a Y to T mutation at a position corresponding to amino acid 182 of SEQ ID NO: 32 (Y182T). In some embodiments, the UCHT1 antigen-binding domain comprises a humanized variant of UCHT1 (huUCHT1). In some embodiments, the UCHT1 antigen-binding domain comprises a humanized variant of UCHT1 comprising a Y to T mutation at a position corresponding to amino acid 177 of SEQ ID NO: 40 (huUCHT1 (Y177T)). In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the antigen-binding domain that binds the protein associated with the TCR complex is an OKT3 antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3). In some embodiments, the antigen-binding domain that binds the protein associated with the TCR complex is a F6A antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A). In some embodiments, the antigen-binding domain that binds the protein associated with the TCR complex is a L2K antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K). In some embodiments, the transmembrane domain is a CD4 transmembrane domain and the cytosolic domain is a CD4 cytosolic domain. In some embodiments, the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain). In some embodiments, the transmembrane domain is a CD8 transmembrane domain and the cytosolic domain is a CD8 cytosolic domain. In some embodiments, the component encoded by (a) and the component encoded by (c) are fused to the component encoded by (b). In some embodiments, the component encoded by (b) and the component encoded by (c) are fused to the component encoded by (a). In some embodiments, at least one linker joins the component encoded by (a) to the component encoded by (b). In some embodiments, the at least one linker is a glycine and/or serine-rich linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker). In some embodiments, the nucleic acid sequence does not encode a co-stimulatory domain. In some embodiments, the nucleic acid sequence does not encode an activation domain. In some embodiments, the nucleic acid sequence further encodes a leader sequence. In some embodiments, the leader sequence comprises an amino acid sequence having at least 80%, at least 85%, at least 90%, at least 95%, at least 96%, at least 97%, at least 98%, at least 99%, or 100% sequence identity with the amino acid sequence of SEQ ID NO: 2 (muIgG leader), SEQ ID NO: 18 (huIgG leader), SEQ ID NO: 20 (huCD8a-1 leader) or SEQ ID NO: 30 (huCD8a-2 leader).

Disclosed herein, in certain embodiments, are nucleic acids encoding GPC3 TAC polypeptides, the nucleic acid sequences comprising a sequence having at least 80% sequence identity with a nucleic acid sequence selected from any one of SEQ ID NOs: 53, 55, 57, 59, 61, 63, 65, 67, 69, 71, 73, or 75.

Disclosed herein, in certain embodiments, are nucleic acids encoding GPC3 TAC polypeptides, the nucleic acid sequences comprising a sequence selected from any one of SEQ ID NOs: 53, 55, 57, 59, 61, 63, 65, 67, 69, 71, 73, or 75.

Disclosed herein, in certain embodiments, are expression vectors comprising a nucleic acid encoding a GPC3 TAC described herein. In some embodiments, the expression vector further comprises a promoter functional in a mammalian cell. In some embodiments, the expression vector is a lentiviral vector. In some embodiments, the lentiviral vector is a VSV-G pseudotyped lentivirus. In some embodiments, the expression vector is a γ retroviral vector. In some embodiments, the γ retroviral vector is a GALV pseudotyped γ-retrovirus.

Disclosed herein, in certain embodiments, are GPC3-TAC polypeptides, comprising: (a) an antigen-binding domain that binds GPC3; (b) an antigen-binding domain that binds a protein associated with a TCR complex; and (c) a TCR co-receptor cytosolic domain and transmembrane domain; wherein component (a), component (b), and component (c) are fused directly to each other, or joined by at least one linker. In some embodiments, the first polynucleotide, the second polynucleotide, and the third polynucleotide are in order. In some embodiments, the antigen-binding domain that binds GPC3-TAC is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds GPC3-TAC is a designed ankyrin repeat (DARPin) polypeptide, a single chain variable fragment (scFv), or a nanobody. In some embodiments, the antigen-binding domain that binds GPC3 is a nanobody. In some embodiments, the antigen-binding domain that binds GPC3 comprises an antigen-binding domain derived from an antibody selected from codrituzumab/GC33, GPC3-C02, A1836A, 1G12, YP8, YP9, or YP9.1. In some embodiments, the antigen-binding domain that binds GPC3 comprises an amino acid sequence having at least 80%, at least 85%, at least 90%, at least 95%, at least 96%, at least 97%, at least 98%, at least 99%, or 100% sequence identity with the amino acid sequence of the antigen-binding domain(s) of codrituzumab/GC33, GPC3-C02, A1836A, 1G12, YP8, YP9, or YP9.1. In some embodiments, the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1. In some embodiments, the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1, and the non-CDR sequences of the antigen-binding domain that binds GPC3 have at least 80%, at least 85%, at least 90%, at least 95%, at least 96%, at least 97%, at least 98%, or at least 99% sequence identity with the non-CDR sequences of the amino acid sequence of codrituzumab/GC33, GPC3-C02, A1836A, 1G12, YP8, YP9, or YP9.1. In some embodiments, the antigen-binding domain comprises: (a) an HCDR1 of SEQ ID NO: 77, an HCDR2 of SEQ ID NO: 78, and an HCDR3 of SEQ ID NO: 79; and a light-chain variable domain comprising an LCDR1 of SEQ ID NO: 80, an LCDR2 of SEQ ID NO: 81, and an LCDR3 of SEQ ID NO: 82; (b) an HCDR1 of SEQ ID NO: 83, an HCDR2 of SEQ ID NO: 84, and an HCDR3 of SEQ ID NO: 85; and a light-chain variable domain comprising an LCDR1 of SEQ ID NO: 86, an LCDR2 of SEQ ID NO: 87, and an LCDR3 of SEQ ID NO: 82; (c) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 80, an LCDR2 of SEQ ID NO: 89, and an LCDR3 of SEQ ID NO: 90; (d) an HCDR1 of SEQ ID NO: 91, an HCDR2 of SEQ ID NO: 92, and an HCDR3 of SEQ ID NO: 93; and a light-chain variable domain comprising an LCDR1 of SEQ ID NO: 86, an LCDR2 of SEQ ID NO: 87, and an LCDR3 of SEQ ID NO: 90; (e) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 94, an LCDR2 of SEQ ID NO: 89, and an LCDR3 of SEQ ID NO: 82; or (f) an HCDR1 of SEQ ID NO: 95, an HCDR2 of SEQ ID NO: 92, and an HCDR3 of SEQ ID NO: 85; and a light-chain variable domain comprising an LCDR1 of SEQ ID NO: 96, an LCDR2 of SEQ ID NO: 87, and an LCDR3 of SEQ ID NO: 82. In some embodiments, the antigen-binding domain comprises an amino acid sequence having at least 80%, at least 85%, at least 90%, at least 95%, at least 96%, at least 97%, at least 98%, at least 99%, or 100% sequence identity with an amino acid sequence selected from SEQ ID NOs: 97-102. In some embodiments, the protein associated with the TCR complex is a CD3 protein. In some embodiments, the CD3 protein is a CD3γ protein, CD3δ protein and/or CD3ε protein. In some embodiments, the CD3 protein is a CD3 protein. In some embodiments, the CD3 protein is a CD3ε protein. In some embodiments, the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds the protein associated with the TCR complex is derived from an antibody selected from UCHT1, OKT3, F6A, and L2K. In some embodiments, the antigen-binding domain that binds the protein associated with the TCR complex is a UCHT1 antigen-binding domain. In some embodiments, the UCHT1 antigen-binding domain is an scFv of UCHT1. In some embodiments, the UCHT1 antigen-binding domain comprises a Y to T mutation at a position corresponding to amino acid 182 of SEQ ID NO: 32 (Y182T). In some embodiments, the UCHT1 antigen-binding domain comprises a humanized variant of UCHT1 (huUCHT1). In some embodiments, the UCHT1 antigen-binding domain comprises a humanized variant of UCHT1 comprising a Y to T mutation at a position corresponding to amino acid 177 of SEQ ID NO: 40 (huUCHT1 (Y177T)). In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the antigen-binding domain that binds the protein associated with the TCR complex is an OKT3 antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3). In some embodiments, the antigen-binding domain that binds the protein associated with the TCR complex is a F6A antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A). In some embodiments, the antigen-binding domain that binds the protein associated with the TCR complex is a L2K antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K). In some embodiments, the transmembrane domain is a CD4 transmembrane domain and the cytosolic domain is a CD4 cytosolic domain. In some embodiments, the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain). In some embodiments, the transmembrane domain is a CD8 transmembrane domain and the cytosolic domain is a CD8 cytosolic domain. In some embodiments, component (a) and component (c) are fused to component (b). In some embodiments, component (b) and component (c) are fused to component (a). In some embodiments, at least one linker joins component (a) to component (b). In some embodiments, the at least one linker is a glycine and/or serine-rich linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker). In some embodiments, the GPC3-TAC does not comprise a co-stimulatory domain. In some embodiments, the GPC3-TAC does not comprise an activation domain.

Disclosed herein, in certain embodiments, are GPC3-TAC polypeptides comprising an amino acid sequence having at least 80% sequence identity with an amino acid sequence selected from any one of SEQ ID NOs: 54, 56, 58, 60, 62, 64, 66, 68, 70, 72, 74, or 76.

Disclosed herein, in certain embodiments, are GPC3-TAC polypeptides comprising an amino acid sequence selected from any one of SEQ ID NOs: 54, 56, 58, 60, 62, 64, 66, 68, 70, 72, 74, or 76.

Disclosed herein, in certain embodiments, are T cells comprising a nucleic acid described herein encoding a GPC3 TAC.

Disclosed herein, in certain embodiments, are T cells comprising an expression vector described herein encoding a GPC3 TAC.

Disclosed herein, in certain embodiments, are T cells comprising a GPC3-TAC polypeptide described herein.

Disclosed herein, in certain embodiments, are pharmaceutical compositions comprising a T cell described herein, and a pharmaceutically acceptable excipient.

Disclosed herein, in certain embodiments, are methods of treating a GPC3-expressing cancer in an individual in need thereof, comprising administering to the individual the pharmaceutical composition described herein. In some embodiments, the cancer is a solid cancer. In some embodiments, the cancer is a liver cancer (for example, HCC), gastric carcinoma, ovarian carcinoma (for example, ovarian clear cell carcinoma), melanoma, colorectal carcinoma, thyroid cancer, squamous cell carcinoma of the lung, hepatoblastoma, nephroblastoma (for example, Wilms tumor), yolk sac tumor, sarcoma, liposarcoma, pediatric embryonal tumor, rhabdoid tumor (for example, rhabdomyosarcoma), or neuroblastoma.

### BRIEF DESCRIPTION OF THE DRAWINGS

The invention can be more completely understood with reference to the following drawings.

FIG. 1 depicts surface expression level of indicated GPC3-TACs as measured by flow cytometry.

FIG. 2 depicts relative GPC3 expression (horizontal axes) with cell counts shown on the vertical axes in indicated cell lines.

FIG. 3 depicts activation of T cells expressing the indicated TACs as measured by up-regulation of CD69 following co-culture with indicated target cells.

FIGS. 4A-4B depict a cell trace assay. FIG. 4A depicts the normalized division indices of T cells expressing the indicated TACs following co-culture with indicated target cells. FIG. 4B depicts representative graphs of cell trace violet (CTV) staining of T cells expressing indicated TACs from FIG. 4A following co-culture with N87^{GPC3} cells.

FIG. 5 depicts fluorescence images of N87^{GPC3-GFPeLuc} target cells co-cultured with T cells expressing the indicated TACs at the start of the experiment (top row) and after 5 days of co-culture (bottom row).

### DETAILED DESCRIPTION

Cancer is a major health challenge. According to the American Cancer Society, more than one million people in the United States are diagnosed with cancer each year. While patients with early stage disease are sometimes treated effectively by conventional therapies (surgery, radiation, chemotherapy), few options are available to patients with advanced disease, and those options are typically palliative in nature.

Active immunotherapy seeks to employ the patient's immune system to clear tumors and offers an option to patients who have failed conventional therapies. Generally, this treatment involves infusing patients with large numbers of tumor-specific T cells. To this point, most engineered T cell therapies involving genetic modification of the T cells yield: (i) forced expression of T cell receptor (TCR); or (ii) a chimeric antigen receptor (CAR) specific for antigen targets on the tumor. To date, the chimeric antigen receptors used for engineering T cells consist of: (i) a targeting domain, usually a single-chain fragment variable (scFv); (ii) a transmembrane domain; and (iii) a cytosolic domain that contains signaling elements from the T cell receptor and associated proteins. Such chimeric antigen receptors have also been referred to as “T-body” or “Chimeric Immune Receptor” (CIR), but currently, most researchers use the term “CAR”. One advantage of the CAR approach is that it allows any patient's immune cells to be targeted against any desirable target in a major histocompatibility complex (MHC) independent manner. This is appealing as MHC presentation is often defective in tumor cells.

CARs are considered in modular terms and scientists have spent considerable time investigating the influence of different cytoplasmic signaling domains on CAR function. Conventional CARs generally share two main components: (i) the CD3 zeta cytoplasmic domain, which contains immunotyrosine activation motifs (ITAMs) critical for T cell activation; and (ii) components of costimulatory receptors that trigger important survival pathways such as the Akt pathway.

The first-generation CARs employed a single signaling domain from either CD3ζ or FcεRIγ. Second-generation CARs combined the signaling domain of CD3ζ with the cytoplasmic domain of costimulatory receptors from either the CD28 or TNFR family of receptors. Most CAR-engineered T cells that are currently being tested in the clinic employ second-generation CARs where CD3ζ is coupled to the cytoplasmic domain of either CD28 or CD137. These second generation CARs have demonstrated anti-tumor activity in CD19-positive tumors. Third-generation CARs combined multiple costimulatory domains, but there is concern that third-generation CARs may lose antigen-specificity.

While CAR-engineered T cells have shown considerable promise in clinical application, they rely on a synthetic method for replacing the native activation signal that is provided by the T cell receptor (TCR). Since this synthetic receptor does not deliver all of the signaling components associated with the TCR (ex. ITAMs on CD3γ, CD3δ, CD3ε), it remains unclear whether the T cells are optimally activated by the CAR or how the CAR activation affects T cell differentiation (ex. progression to memory). Furthermore, since the CAR signaling domains are disconnected from their natural regulatory partners by the very nature of the CAR structure, there is an inherent risk that CARs may lead to a low-level of constitutive activation, which could result in off-target toxicities. Therefore, the synthetic nature of the prototypic CAR may disrupt canonical mechanisms that limit TCR activation, and may underpin the severe toxicity often associated with therapeutic doses of conventional CAR T cells.

Given these limitations, it is preferable to re-direct T cells to attack tumors via their natural TCR. An alternate chimeric receptor, termed a T cell Antigen Coupler (TAC or TAC) receptor, has been developed which employs a distinct biology to direct the T cell to attack tumors. While the CAR is a fully synthetic receptor that stitches together components of T cell receptor (TCR) signaling complex, the TAC receptor re-directs the TCR towards tumor targets and recapitulates the native TCR signaling structure. For example, in some embodiments, the TACs disclosed herein activate natural Major Histocompatibility complex (MHC) signaling through the T cell receptor (TCR), while retaining MHC-unrestricted targeting. Further, the TACs disclosed herein recruit the T Cell Receptor (TCR) in combination with co-receptor stimulation. Moreover, in some embodiments, TACs disclosed herein show enhanced activity and safety.

### Certain Terminology

The term “antigen-binding domain,” refers to any substance or molecule that binds, directly or indirectly, to a target (e.g., GPC3). Antigen-binding domains include antibodies or fragments thereof, peptides, peptidomimetics, proteins, glycoproteins, proteoglycans, carbohydrates, lipids, nucleic acids, or small molecules that bind to a target.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The term “T cell” as used herein refers to a type of lymphocyte that plays a central role in cell-mediated immunity. T cells, also referred to as T lymphocytes, are distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells and natural killer T cells.

The term “γδ T cell” or “gamma delta T cell” or “gd T cell” as used herein refers to any lymphocyte having a γδ T cell receptor (TCR) on its surface, including one γ-chain and one δ-chain.

The term “T cell antigen coupler” or TAC is used interchangeably with “trifunctional T cell antigen coupler” or Tri-TAC and refers to an engineered nucleic acid construct or polypeptide comprising (a) an antigen-binding domain that binds a target, (b) an antigen-binding domain that binds a protein associated with a T cell receptor (TCR) complex, and (c) a T cell receptor signaling domain.

The term “polynucleotide” and/or “nucleic acid sequence” and/or “nucleic acid” as used herein refers to a sequence of nucleoside or nucleotide monomers consisting of bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The term “isolated polynucleotide” or “isolated nucleic acid sequence” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and is either double stranded or single stranded, and represents the sense or antisense strand. Further, the term “nucleic acid” includes the complementary nucleic acid sequences.

The term “recombinant nucleic acid” or “engineered nucleic acid” as used herein refers to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids.

The terms “peptide”, “polypeptide,” and “protein” as used herein mean a chain of amino acids. The term protein as used herein further means a large molecule comprising one or more chains of amino acids and, in some embodiments, is a fragment or domain of a protein or a full length protein. Furthermore, as used herein, the term protein either refers to a linear chain of amino acids or to a chain of amino acids that has been processed and folded into a functional protein. The protein structure is divided into four distinct levels: (1) primary structure—referring to the sequence of amino acids in the polypeptide chain, (2) secondary structure—referring to the regular local sub-structures on the polypeptide backbone chain, such as α-helix and β-sheets, (3) tertiary structure—referring to the three-dimensional structure if monomeric and multimeric protein molecules, and (4) quaternary structure—referring to the three-dimensional structure comprising the aggregation of two or more individual polypeptide chains that operate as a single functional unit. The use of peptide or polypeptide herein does not mean that the chain of amino acids is not also a protein (i.e., a chain of amino acids having a secondary, tertiary or quaternary structure).

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that is used to deliver a nucleic acid to the inside of a cell. In some embodiments, a vector is an expression vector comprising expression control sequences (for example, a promoter) operatively linked to a nucleic acid to be expressed in a cell. Vectors known in the art include, but are not limited to, plasmids, phages, cosmids and viruses.

The term “tumor antigen” or “tumor associated antigen” as used herein refers to an antigenic substance produced in tumor cells that triggers an immune response in a host (e.g. which is presented by MHC complexes). In some embodiments, a tumor antigen is on the surface of a tumor cell.

As used herein, the term “transmembrane and cytosolic domain” refers to a polypeptide that comprises a transmembrane domain and a cytosolic domain of a protein associated with the T cell receptor (TCR) complex. In some embodiments, such transmembrane and cytosolic domain may include, but is not limited to, protein domains that (a) associate with the lipid raft and/or (b) bind Lck.

A “TCR co-receptor” as used herein, refers to a molecule that assists the T cell receptor (TCR) in communicating with an antigen-presenting cell and may be considered part of the first signal that leads to the activation of the TCR. Examples of TCR co-receptors include, but are not limited to, CD4, LAG3, and CD8.

A “TCR co-stimulator” or “co-stimulatory domain” as used herein, refers to a molecule that enhances the response of a T cell to an antigen and may be considered as the second signal that leads to the activation of the TCR. Examples of TCR co-stimulators include, but are not limited to, ICOS, CD27, CD28, 4-1BB (CD 137), OX40 (CD134), CD30, CD40, lymphocyte fiction-associated antigen 1 (LFA-1), CD2, CD7, LIGHT, NKG2C, B7-H3, and a ligand that specifically binds CD83.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g. cancer). The term “therapeutic effect” refers to the reduction, elimination, or prevention of the disease, symptoms of the disease, or side effects of the disease in the subject.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

As used herein, the term “selective binding” refers to the higher affinity with which a molecule (e.g. protein such as an antigen-binding domain of TAC) binds its target molecule (e.g. target antigen such as GPC3) over other molecules. Unless indicated otherwise, the terms “selective binding” and “specific binding” are used interchangeably herein.

As used herein, the term GPC3 means the protein glypican 3. GPC3 is one of several glypicans, each of which consists of a core protein attached to long sugar molecules called heparan sulfate chains. Glypicans are anchored to the outer cell membrane, where they interact with a variety of other proteins outside the cell. Glypican 3 interacts with other proteins at the surface of cells to restrain cell proliferation. Specifically, GPC3 blocks the hedgehog signaling pathway. Glypican 3 may act as a tumor suppressor. Glypican 3 may also induce apoptosis. Although GPC3 is known primarily as an inhibitor of cell growth and cell division, in some tissues it appears to have the opposite effect. Research suggests that in certain types of cells, such as cells in the liver, glypican 3 may interact with proteins called growth factors to promote cell growth and cell division.

### T Cell Antigen Couplers (TACs)

Disclosed herein, in certain embodiments, are nucleic acids encoding GPC3 T cell-antigen coupler (TAC) polypeptides. In some embodiments, the nucleic acids encoding the GPC3 TAC comprise: (a) a first polynucleotide encoding an antigen-binding domain that binds GPC3; (b) a second polynucleotide encoding an antigen-binding domain that binds the TCR complex; and (c) a third polynucleotide encoding a transmembrane domain and cytosolic domain. In some embodiments, the nucleic acids comprise, in order (e.g., from 5′ to 3′): (a) the first polynucleotide; (b) the second polynucleotide; and (c) the third polynucleotide encoding a TCR co-receptor cytosolic domain and transmembrane domain. In some embodiments, the nucleic acids encoding the GPC3 TAC do not encode a co-stimulatory domain. In some embodiments, the nucleic acids encoding the GPC3 TAC do not encode a co-activation domain.

Further disclosed herein, in certain embodiments, are GPC3 T cell-antigen coupler (TAC) polypeptides. In some embodiments, the GPC3 TAC polypeptides comprise: (a) an antigen-binding domain that binds GPC3; (b) an antigen-binding domain that binds the TCR complex; and (c) a transmembrane domain and cytosolic domain. In some embodiments, the GPC3 TAC polypeptides comprise, in order (e.g., from N-terminus to C-terminus) (a) the antigen-binding domain that binds GPC3; (b) the antigen-binding domain that binds the TCR complex; and (c) the transmembrane domain and cytosolic domain. In some embodiments, the GPC3 TAC polypeptides do not include a co-stimulatory domain. In some embodiments, the GPC3 TAC polypeptides do not include a co-activation domain.

Further disclosed herein, in certain embodiments, are expression vectors comprising a nucleic acid encoding a GPC3 TAC polypeptide as described herein.

Further disclosed herein, in certain embodiments, are T cells comprising a nucleic acid encoding a GPC3 TAC polypeptide as described herein, T cells comprising an expression vector encoding a GPC3 TAC polypeptide as described herein, or T cells comprising a GPC3 TAC polypeptide as described herein.

Further disclosed herein, in certain embodiments, are methods of treating a cancer in an individual in need thereof, comprising administering to the individual a T cell comprising a GPC3 T cell-antigen coupler (TAC) polypeptide as described herein.

### GPC3 Antigen-Binding Domain

In certain embodiments, the GPC3 TAC polypeptide comprises a GPC3 antigen-binding domain. In some embodiments, the GPC3 antigen-binding domain selectively binds GPC3. In some embodiments, the GPC3 antigen-binding domain binds to GPC3 on a target cell. In some embodiments, a target cell is a cell associated with a disease state, including, but not limited to, cancer. In some embodiments, a target cell is a tumor cell.

In some embodiments, the GPC3 antigen-binding domain is an antibody or a fragment thereof. In some embodiments, the GPC3 antigen-binding domain is selected from single chain antibodies (e.g., single-chain fragment variable antibodies (scFvs)), single domain antibodies (e.g., heavy-chain-only antibodies (VHH), shark heavy-chain-only antibodies (VNAR)), nanobodies, diabodies, minibodies, Fab fragments, Fab′ fragments, F(ab′)₂ fragments, or Fv fragments that bind to GPC3.

In some embodiments, the GPC3 antigen-binding domain is selected from ankyrin repeat proteins (DARPins), affibodies, adnectins, affilins, phylomers, fynomers, affimers, peptide aptamers, lectins, knottins, centyrins, anticalins, peptides, peptidomimetics, proteins, glycoproteins, or proteoglycans that bind to GPC3, or naturally occurring ligands for GPC3. In some embodiments, the GPC3 antigen-binding domain is a non-protein compound that binds to GPC3, including but not limited to carbohydrates, lipids, nucleic acids, or small molecules.

In some embodiments, the GPC3 antigen-binding domain is a designed ankyrin repeat (DARPin) targeted to GPC3. In some embodiments, the GPC3 antigen-binding domain is a single-chain variable fragment (ScFv) targeted to GPC3. In some embodiments, the GPC3 antigen-binding domain is a nanobody targeted to GPC3.

In some embodiments, the GPC3 antigen binding domain is codrituzumab/GC33, or a fragment thereof. In some embodiments, the GPC3 antigen binding domain comprises the heavy chain variable domain of codrituzumab/GC33. In some embodiments, the GPC3 antigen binding domain comprises the light chain variable domain of codrituzumab/GC33. In some embodiments, the GPC3 antigen binding domain comprises the heavy chain variable domain of codrituzumab/GC33 and the light chain variable domain of codrituzumab/GC33. In some embodiments, the GPC3 antigen binding domain is an scFv comprising the heavy chain variable domain of codrituzumab/GC33 and the light chain variable domain of codrituzumab/GC33.

In some embodiments, the GPC3 antigen binding domain is GPC3-C02, or a fragment thereof. In some embodiments, the GPC3 antigen binding domain comprises the heavy chain variable domain of GPC3-C02. In some embodiments, the GPC3 antigen binding domain comprises the light chain variable domain of GPC3-C02. In some embodiments, the GPC3 antigen binding domain comprises the heavy chain variable domain of GPC3-C02 and the light chain variable domain of GPC3-C02. In some embodiments, the GPC3 antigen binding domain is an scFv comprising the heavy chain variable domain of GPC3-C02 and the light chain variable domain of GPC3-C02.

In some embodiments, the GPC3 antigen binding domain is A1836A, or a fragment thereof. In some embodiments, the GPC3 antigen binding domain comprises the heavy chain variable domain of A1836A. In some embodiments, the GPC3 antigen binding domain comprises the light chain variable domain of A1836A. In some embodiments, the GPC3 antigen binding domain comprises the heavy chain variable domain of A1836A and the light chain variable domain of A1836A. In some embodiments, the GPC3 antigen binding domain is an scFv comprising the heavy chain variable domain of A1836A and the light chain variable domain of A1836A.

In some embodiments, the GPC3 antigen binding domain is 1G12, or a fragment thereof. In some embodiments, the GPC3 antigen binding domain comprises the heavy chain variable domain of 1G12. In some embodiments, the GPC3 antigen binding domain comprises the light chain variable domain of 1G12. In some embodiments, the GPC3 antigen binding domain comprises the heavy chain variable domain of 1G12 and the light chain variable domain of 1G12. In some embodiments, the GPC3 antigen binding domain is an scFv comprising the heavy chain variable domain of 1G12 and the light chain variable domain of 1G12.

In some embodiments, the GPC3 antigen binding domain is YP8, or a functional fragment thereof. In some embodiments, the GPC3 antigen binding domain comprises a heavy chain variable domain comprising a heavy chain complementarity-determining region 1 (HCDR1) of SEQ ID NO: 77, a heavy chain complementarity-determining region 2 (HCDR2) of SEQ ID NO: 78, and a heavy chain complementarity-determining region 3 (HCDR3) of SEQ ID NO: 79; and a light-chain variable domain comprising a light chain complementarity-determining region 1 (LCDR1) of SEQ ID NO: 80, a light chain complementarity-determining region 2 (LCDR2) of SEQ ID NO: 81, and a light chain complementarity-determining region 3 (LCDR3) of SEQ ID NO: 82. In some embodiments, the GPC3 antigen binding domain comprises a heavy chain variable domain comprising an HCDR1 of SEQ ID NO: 83, an HCDR2 of SEQ ID NO: 84, and an HCDR3 of SEQ ID NO: 85; and a light-chain variable domain comprising an LCDR1 of SEQ ID NO: 86, an LCDR2 of SEQ ID NO: 87, and an LCDR3 of SEQ ID NO: 82.

In some embodiments, the GPC3 antigen binding domain is YP9, or a functional fragment thereof. In some embodiments, the GPC3 antigen binding domain comprises a heavy chain variable domain comprising an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 80, an LCDR2 of SEQ ID NO: 89, and an LCDR3 of SEQ ID NO: 90. In some embodiments, the GPC3 antigen binding domain comprises a heavy chain variable domain comprising an HCDR1 of SEQ ID NO: 91, an HCDR2 of SEQ ID NO: 92, and an HCDR3 of SEQ ID NO: 93; and a light-chain variable domain comprising an LCDR1 of SEQ ID NO: 86, an LCDR2 of SEQ ID NO: 87, and an LCDR3 of SEQ ID NO: 90.

In some embodiments, the GPC3 antigen binding domain is YP9.1, or a functional fragment thereof. In some embodiments, the GPC3 antigen binding domain comprises a heavy chain variable domain comprising an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 94, an LCDR2 of SEQ ID NO: 89, and an LCDR3 of SEQ ID NO: 82. In some embodiments, the GPC3 antigen binding domain comprises a heavy chain variable domain comprising an HCDR1 of SEQ ID NO: 95, an HCDR2 of SEQ ID NO: 92, and an HCDR3 of SEQ ID NO: 85; and a light-chain variable domain comprising an LCDR1 of SEQ ID NO: 96, an LCDR2 of SEQ ID NO: 87, and an LCDR3 of SEQ ID NO: 82.

Amino acid sequences of exemplary CDRs of antigen-binding domains that bind GPC3 are provided in Table 1.

TABLE 1
Table of Sequences

| SEQ ID NO | Description | Nucleotide/Amino Acid |
|---|---|---|
| SEQ ID NO: 77 | YP8 HCDR1 (Kabat) | Amino Acid |
| SEQ ID NO: 78 | YP8 HCDR2 (Kabat) | Amino Acid |
| SEQ ID NO: 79 | YP8 HCDR3 (Kabat) | Amino Acid |
| SEQ ID NO: 80 | YP8 LCDR1 (Kabat) | Amino Acid |
| SEQ ID NO: 81 | YP8 LCDR2 (Kabat) | Amino Acid |
| SEQ ID NO: 82 | YP8 LCDR3 (Kabat) | Amino Acid |
| SEQ ID NO: 83 | YP8 HCDR1 (IMGT) | Amino Acid |
| SEQ ID NO: 84 | YP8 HCDR2 (IMGT) | Amino Acid |
| SEQ ID NO: 85 | YP8 HCDR3 (IMGT) | Amino Acid |
| SEQ ID NO: 86 | YP8 LCDR1 (IMGT) | Amino Acid |
| SEQ ID NO: 87 | YP8 LCDR2 (IMGT) | Amino Acid |
| SEQ ID NO: 82 | YP8 LCDR3 (IMGT) | Amino Acid |
| SEQ ID NO: 77 | YP9 HCDR1 (Kabat) | Amino Acid |
| SEQ ID NO: 88 | YP9 HCDR2 (Kabat) | Amino Acid |
| SEQ ID NO: 79 | YP9 HCDR3 (Kabat) | Amino Acid |
| SEQ ID NO: 80 | YP9 LCDR1 (Kabat) | Amino Acid |
| SEQ ID NO: 89 | YP9 LCDR2 (Kabat) | Amino Acid |
| SEQ ID NO: 90 | YP9 LCDR3 (Kabat) | Amino Acid |
| SEQ ID NO: 91 | YP9 HCDR1 (IMGT) | Amino Acid |
| SEQ ID NO: 92 | YP9 HCDR2 (IMGT) | Amino Acid |
| SEQ ID NO: 93 | YP9 HCDR3 (IMGT) | Amino Acid |
| SEQ ID NO: 86 | YP9 LCDR1 (IMGT) | Amino Acid |
| SEQ ID NO: 87 | YP9 LCDR2 (IMGT) | Amino Acid |
| SEQ ID NO: 90 | YP9 LCDR3 (IMGT) | Amino Acid |
| SEQ ID NO: 77 | YP9.1 HCDR1 (Kabat) | Amino Acid |
| SEQ ID NO: 88 | YP9.1 HCDR2 (Kabat) | Amino Acid |
| SEQ ID NO: 79 | YP9.1 HCDR3 (Kabat) | Amino Acid |
| SEQ ID NO: 94 | YP9.1 LCDR1 (Kabat) | Amino Acid |
| SEQ ID NO: 89 | YP9.1 LCDR2 (Kabat) | Amino Acid |
| SEQ ID NO: 82 | YP9.1 LCDR3 (Kabat) | Amino Acid |
| SEQ ID NO: 95 | YP9.1 HCDR1 (IMGT) | Amino Acid |
| SEQ ID NO: 92 | YP9.1 HCDR2 (IMGT) | Amino Acid |
| SEQ ID NO: 85 | YP9.1 HCDR3 (IMGT) | Amino Acid |
| SEQ ID NO: 96 | YP9.1 LCDR1 (IMGT) | Amino Acid |
| SEQ ID NO: 87 | YP9.1 LCDR2 (IMGT) | Amino Acid |
| SEQ ID NO: 82 | YP9.1 LCDR3 (IMGT) | Amino Acid |

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 97. In some embodiments the GPC3 antigen binding domain comprises an amino acid sequence at least 80% identical to the amino acid sequence of SEQ ID NO: 97. In some embodiments the GPC3 antigen binding domain comprises an amino acid sequence at least 85% identical to the amino acid sequence of SEQ ID NO: 97. In some embodiments the GPC3 antigen binding domain comprises an amino acid sequence at least 90% identical to the amino acid sequence of SEQ ID NO: 97. In some embodiments the GPC3 antigen binding domain comprises an amino acid sequence at least 95% identical to the amino acid sequence of SEQ ID NO: 97. In some embodiments the GPC3 antigen binding domain comprises an amino acid sequence at least 96% identical to the amino acid sequence of SEQ ID NO: 97. In some embodiments the GPC3 antigen binding domain comprises an amino acid sequence at least 97% identical to the amino acid sequence of SEQ ID NO: 97. In some embodiments the GPC3 antigen binding domain comprises an amino acid sequence at least 98% identical to the amino acid sequence of SEQ ID NO: 97. In some embodiments the GPC3 antigen binding domain comprises an amino acid sequence at least 99% identical to the amino acid sequence of SEQ ID NO: 97. In some embodiments the GPC3 antigen binding domain comprises the amino acid sequence of SEQ ID NO: 97.

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 98. In some embodiments the GPC3 antigen binding domain comprises an amino acid sequence at least 80% identical to the amino acid sequence of SEQ ID NO: 98. In some embodiments the GPC3 antigen binding domain comprises an amino acid sequence at least 85% identical to the amino acid sequence of SEQ ID NO: 98. In some embodiments the GPC3 antigen binding domain comprises an amino acid sequence at least 90% identical to the amino acid sequence of SEQ ID NO: 98. In some embodiments the GPC3 antigen binding domain comprises an amino acid sequence at least 95% identical to the amino acid sequence of SEQ ID NO: 98. In some embodiments the GPC3 antigen binding domain comprises an amino acid sequence at least 96% identical to the amino acid sequence of SEQ ID NO: 98. In some embodiments the GPC3 antigen binding domain comprises an amino acid sequence at least 97% identical to the amino acid sequence of SEQ ID NO: 98. In some embodiments the GPC3 antigen binding domain comprises an amino acid sequence at least 98% identical to the amino acid sequence of SEQ ID NO: 98. In some embodiments the GPC3 antigen binding domain comprises an amino acid sequence at least 99% identical to the amino acid sequence of SEQ ID NO: 98. In some embodiments the GPC3 antigen binding domain comprises the amino acid sequence of SEQ ID NO: 98.

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 99. In some embodiments the GPC3 antigen binding domain comprises an amino acid sequence at least 80% identical to the amino acid sequence of SEQ ID NO: 99. In some embodiments the GPC3 antigen binding domain comprises an amino acid sequence at least 85% identical to the amino acid sequence of SEQ ID NO: 99. In some embodiments the GPC3 antigen binding domain comprises an amino acid sequence at least 90% identical to the amino acid sequence of SEQ ID NO: 99. In some embodiments the GPC3 antigen binding domain comprises an amino acid sequence at least 95% identical to the amino acid sequence of SEQ ID NO: 99. In some embodiments the GPC3 antigen binding domain comprises an amino acid sequence at least 96% identical to the amino acid sequence of SEQ ID NO: 99. In some embodiments the GPC3 antigen binding domain comprises an amino acid sequence at least 97% identical to the amino acid sequence of SEQ ID NO: 99. In some embodiments the GPC3 antigen binding domain comprises an amino acid sequence at least 98% identical to the amino acid sequence of SEQ ID NO: 99. In some embodiments the GPC3 antigen binding domain comprises an amino acid sequence at least 99% identical to the amino acid sequence of SEQ ID NO: 99. In some embodiments the GPC3 antigen binding domain comprises the amino acid sequence of SEQ ID NO: 99.

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 100. In some embodiments the GPC3 antigen binding domain comprises an amino acid sequence at least 80% identical to the amino acid sequence of SEQ ID NO: 100. In some embodiments the GPC3 antigen binding domain comprises an amino acid sequence at least 85% identical to the amino acid sequence of SEQ ID NO: 100. In some embodiments the GPC3 antigen binding domain comprises an amino acid sequence at least 90% identical to the amino acid sequence of SEQ ID NO: 100. In some embodiments the GPC3 antigen binding domain comprises an amino acid sequence at least 95% identical to the amino acid sequence of SEQ ID NO: 100. In some embodiments the GPC3 antigen binding domain comprises an amino acid sequence at least 96% identical to the amino acid sequence of SEQ ID NO: 100. In some embodiments the GPC3 antigen binding domain comprises an amino acid sequence at least 97% identical to the amino acid sequence of SEQ ID NO: 100. In some embodiments the GPC3 antigen binding domain comprises an amino acid sequence at least 98% identical to the amino acid sequence of SEQ ID NO: 100. In some embodiments the GPC3 antigen binding domain comprises an amino acid sequence at least 99% identical to the amino acid sequence of SEQ ID NO: 100. In some embodiments the GPC3 antigen binding domain comprises the amino acid sequence of SEQ ID NO: 100.

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 101. In some embodiments the GPC3 antigen binding domain comprises an amino acid sequence at least 80% identical to the amino acid sequence of SEQ ID NO: 101. In some embodiments the GPC3 antigen binding domain comprises an amino acid sequence at least 85% identical to the amino acid sequence of SEQ ID NO: 101. In some embodiments the GPC3 antigen binding domain comprises an amino acid sequence at least 90% identical to the amino acid sequence of SEQ ID NO: 101. In some embodiments the GPC3 antigen binding domain comprises an amino acid sequence at least 95% identical to the amino acid sequence of SEQ ID NO: 101. In some embodiments the GPC3 antigen binding domain comprises an amino acid sequence at least 96% identical to the amino acid sequence of SEQ ID NO: 101. In some embodiments the GPC3 antigen binding domain comprises an amino acid sequence at least 97% identical to the amino acid sequence of SEQ ID NO: 101. In some embodiments the GPC3 antigen binding domain comprises an amino acid sequence at least 98% identical to the amino acid sequence of SEQ ID NO: 101. In some embodiments the GPC3 antigen binding domain comprises an amino acid sequence at least 99% identical to the amino acid sequence of SEQ ID NO: 101. In some embodiments the GPC3 antigen binding domain comprises the amino acid sequence of SEQ ID NO: 101.

In some embodiments the GPC3 antigen binding domain comprises an amino acid sequence at least 80%, at least 85%, at least 90%, at least 95%, at least 96%, at least 97%, at least 98%, at least 99%, or 100% identical to the amino acid sequence of SEQ ID NO: 102. In some embodiments the GPC3 antigen binding domain comprises an amino acid sequence at least 80% identical to the amino acid sequence of SEQ ID NO: 102. In some embodiments the GPC3 antigen binding domain comprises an amino acid sequence at least 85% identical to the amino acid sequence of SEQ ID NO: 102. In some embodiments the GPC3 antigen binding domain comprises an amino acid sequence at least 90% identical to the amino acid sequence of SEQ ID NO: 102. In some embodiments the GPC3 antigen binding domain comprises an amino acid sequence at least 95% identical to the amino acid sequence of SEQ ID NO: 102. In some embodiments the GPC3 antigen binding domain comprises an amino acid sequence at least 96% identical to the amino acid sequence of SEQ ID NO: 102. In some embodiments the GPC3 antigen binding domain comprises an amino acid sequence at least 97% identical to the amino acid sequence of SEQ ID NO: 102. In some embodiments the GPC3 antigen binding domain comprises an amino acid sequence at least 98% identical to the amino acid sequence of SEQ ID NO: 102. In some embodiments the GPC3 antigen binding domain comprises an amino acid sequence at least 99% identical to the amino acid sequence of SEQ ID NO: 102. In some embodiments the GPC3 antigen binding domain comprises the amino acid sequence of SEQ ID NO: 102.

Amino acid sequences of exemplary antigen-binding domains that bind GPC3 are provided in Table 2.

TABLE 2
Table of Sequences

| SEQ ID NO | Description | Nucleotide/Amino Acid |
|---|---|---|
| SEQ ID NO: 97 | YP8 VH-VL | Amino Acid |
| SEQ ID NO: 98 | YP8 VL-VH | Amino Acid |
| SEQ ID NO: 99 | YP9 VH-VL | Amino Acid |
| SEQ ID NO: 100 | YP9 VL-VH | Amino Acid |
| SEQ ID NO: 101 | YP9.1 VH-VL | Amino Acid |
| SEQ ID NO: 102 | YP9.1 VL-VH | Amino Acid |

### TCR Complex Protein Antigen-Binding Domain

In certain embodiments, the GPC3 TAC comprises an antigen-binding domain that binds a protein associated with the TCR complex. A “TCR complex protein antigen-binding domain,” also referred to as a “TCR complex antigen-binding domain,” “antigen-binding domain that binds the TCR complex,” or “antigen-binding domain that binds a protein associated with the TCR complex,” refers to any substance or molecule that binds, directly or indirectly, toa protein associated with a TCR complex. In some embodiments, the antigen-binding domain that binds a protein associated with a TCR complex selectively binds to a protein of the TCR. In some embodiments, the antigen-binding domain that binds a protein associated with a TCR complex comprises a substance that specifically binds to a protein of the TCR.

In some embodiments, the TCR complex protein antigen-binding domain is selected from antibodies or fragments thereof, for example, single chain antibodies (e.g., single-chain fragment variable antibodies (scFvs)), single domain antibodies (e.g., heavy-chain-only antibodies (VHH), shark heavy-chain-only antibodies (VNAR)), nanobodies, diabodies, minibodies, Fab fragments, Fab′ fragments, F(ab′)₂ fragments, or Fv fragments that bind to a protein of the TCR. In some embodiments, the TCR complex protein antigen-binding domain is selected from ankyrin repeat proteins (DARPins), affibodies, adnectins, affilins, phylomers; fynomers, affimers, peptide aptamers, lectins, knottins, centyrins, anticalins, peptides, peptidomimetics, proteins, glycoproteins, or proteoglycans that bind to a protein of the TCR, or naturally occurring ligands for a protein of the TCR. In some embodiments, the TCR complex protein antigen-binding domain is a non-protein compound that binds to a protein of the TCR, including but not limited to carbohydrates, lipids, nucleic acids, or small molecules. In some embodiments, the TCR complex protein antigen-binding domain is a designed ankyrin repeat (DARPin) targeted to a protein of the TCR. In some embodiments, the TCR complex protein antigen-binding domain is a single-chain variable fragment (ScFv) targeted to a protein of the TCR. In some embodiments, the TCR complex protein antigen-binding domain is a nanobody targeted to a protein of the TCR.

Proteins associated with the TCR include, but are not limited, to the TCR alpha (α) chain, TCR beta (β) chain, TCR gamma (γ) chain, TCR delta (δ) chain, CD3γ chain, CD3δ chain and CD3ε chains. In some embodiments, an antigen-binding domain that binds a protein associated with the TCR complex is an antibody to the TCR alpha (α) chain, TCR beta (β) chain, TCR gamma (γ) chain, TCR delta (δ) chain, CD3γ chain, CD3δ chain and/or CD3ε chain. In some embodiments, the protein associated with a TCR complex is CD3. In some embodiments, the protein associated with a TCR complex is CD3ε. In some embodiments, the antigen-binding domain that binds CD3 is an antibody, for example, a single chain antibody, for example a single-chain variable fragment (scFv). Examples of CD3 antibodies, include, but are not limited to, UCHT1, OKT3, F6A, L2K, muromonab, otelixizumab, teplizumab, visilizumab, CD3-12, MEM-57, 4D10A6, CD3D, or TR66.

In some embodiments, the antigen-binding domain that binds the TCR complex is UCHT1, or a variant thereof. In some embodiments, the UCHT1 antigen-binding domain is encoded by SEQ ID NO: 31. In some embodiments, the UCHT1 antigen-binding domain comprises SEQ ID NO: 32. In some embodiments, the UCHT1 antigen-binding domain is mutated. In some embodiments, the UCHT1 antigen-binding domain comprises a Y to T mutation at a position corresponding to amino acid 182 of SEQ ID NO: 32 (Y182T). In some embodiments, the UCHT1 (Y182T) antigen-binding domain is encoded by SEQ ID NO: 43. In some embodiments, the UCHT1 (Y182T) antigen-binding domain comprises SEQ ID NO: 44. In some embodiments, the antigen-binding domain that binds the TCR complex is a humanized UCHT1 (huUCHT1). In some embodiments, the huUCHT1 antigen-binding domain is encoded by SEQ ID NO: 39. In some embodiments, the huUCHT1 antigen-binding domain comprises SEQ ID NO: 40. In some embodiments, the huUCHT1 has a Y to T mutation at a position corresponding to amino acid 177 of SEQ ID NO: 40 (Y177T). In some embodiments, the huUCHT1 (Y177T) antigen-binding domain is encoded by SEQ ID NO: 41. In some embodiments, the huUCHT1 antigen-binding domain comprises SEQ ID NO: 42.

In some embodiments, the polynucleotide encoding the antigen-binding domain that binds the protein associated with the TCR complex comprises a nucleotide sequence having at least 70% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 31 (UCHT1). In some embodiments, the polynucleotide encoding the antigen-binding domain that binds the protein associated with the TCR complex comprises the nucleotide sequence of SEQ ID NO: 31 (UCHT1).

In some embodiments, the antigen-binding domain that binds the protein associated with the TCR complex comprises an amino acid sequence having at least 70% sequence identity with the amino acid sequence of SEQ ID NO: 32 (UCHT1). In some embodiments, the antigen-binding domain that binds the protein associated with the TCR complex comprises an amino acid sequence having at least 75% sequence identity with the amino acid sequence of SEQ ID NO: 32 (UCHT1). In some embodiments, the antigen-binding domain that binds the protein associated with the TCR complex comprises an amino acid sequence having at least 80% sequence identity with the amino acid sequence of SEQ ID NO: 32 (UCHT1). In some embodiments, the antigen-binding domain that binds the protein associated with the TCR complex comprises an amino acid sequence having at least 85% sequence identity with the amino acid sequence of SEQ ID NO: 32 (UCHT1). In some embodiments, the antigen-binding domain that binds the protein associated with the TCR complex comprises an amino acid sequence having at least 90% sequence identity with the amino acid sequence of SEQ ID NO: 32 (UCHT1). In some embodiments, the antigen-binding domain that binds the protein associated with the TCR complex comprises an amino acid sequence having at least 95% sequence identity with the amino acid sequence of SEQ ID NO: 32 (UCHT1). In some embodiments, the antigen-binding domain that binds the protein associated with the TCR complex comprises an amino acid sequence having at least 96% sequence identity with the amino acid sequence of SEQ ID NO: 32 (UCHT1). In some embodiments, the antigen-binding domain that binds the protein associated with the TCR complex comprises an amino acid sequence having at least 97% sequence identity with the amino acid sequence of SEQ ID NO: 32 (UCHT1). In some embodiments, the antigen-binding domain that binds the protein associated with the TCR complex comprises an amino acid sequence having at least 98% sequence identity with the amino acid sequence of SEQ ID NO: 32 (UCHT1). In some embodiments, the antigen-binding domain that binds the protein associated with the TCR complex comprises an amino acid sequence having at least 99% sequence identity with the amino acid sequence of SEQ ID NO: 32 (UCHT1). In some embodiments, the antigen-binding domain that binds the protein associated with the TCR complex comprises the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 32 (UCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 75% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 80% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 85% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 90% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 95% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 96% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 97% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 98% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 99% sequence identity with the nucleotide sequence of SEQ ID NO: 43 (UCHT1 (Y182T)). In some embodiments, the polynucleotide encoding the antigen-binding domain that binds the protein associated with the TCR complex comprises the nucleotide sequence of SEQ ID NO: 43 (UCHT1 (Y182T)).

In some embodiments, the antigen-binding domain that binds the protein associated with the TCR complex comprises an amino acid sequence having at least 70% sequence identity with the amino acid sequence of SEQ ID NO: 44 (UCHT1 (Y182T)). In some embodiments, the antigen-binding domain that binds the protein associated with the TCR complex comprises an amino acid sequence having at least 75% sequence identity with the amino acid sequence of SEQ ID NO: 44 (UCHT1 (Y182T)). In some embodiments, the antigen-binding domain that binds the protein associated with the TCR complex comprises an amino acid sequence having at least 80% sequence identity with the amino acid sequence of SEQ ID NO: 44 (UCHT1 (Y182T)). In some embodiments, the antigen-binding domain that binds the protein associated with the TCR complex comprises an amino acid sequence having at least 85% sequence identity with the amino acid sequence of SEQ ID NO: 44 (UCHT1 (Y182T)). In some embodiments, the antigen-binding domain that binds the protein associated with the TCR complex comprises an amino acid sequence having at least 90% sequence identity with the amino acid sequence of SEQ ID NO: 44 (UCHT1 (Y182T)). In some embodiments, the antigen-binding domain that binds the protein associated with the TCR complex comprises an amino acid sequence having at least 95% sequence identity with the amino acid sequence of SEQ ID NO: 44 (UCHT1 (Y182T)). In some embodiments, the antigen-binding domain that binds the protein associated with the TCR complex comprises an amino acid sequence having at least 96% sequence identity with the amino acid sequence of SEQ ID NO: 44 (UCHT1 (Y182T)). In some embodiments, the antigen-binding domain that binds the protein associated with the TCR complex comprises an amino acid sequence having at least 97% sequence identity with the amino acid sequence of SEQ ID NO: 44 (UCHT1 (Y182T)). In some embodiments, the antigen-binding domain that binds the protein associated with the TCR complex comprises an amino acid sequence having at least 98% sequence identity with the amino acid sequence of SEQ ID NO: 44 (UCHT1 (Y182T)). In some embodiments, the antigen-binding domain that binds the protein associated with the TCR complex comprises an amino acid sequence having at least 99% sequence identity with the amino acid sequence of SEQ ID NO: 44 (UCHT1 (Y182T)). In some embodiments, the antigen-binding domain that binds the protein associated with the TCR complex comprises the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 44 (UCHT1 (Y182T)).

In some embodiments, the polynucleotide encoding the antigen-binding domain that binds the protein associated with the TCR complex comprises a nucleotide sequence having at least 70% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 39 (huUCHT1). In some embodiments, the polynucleotide encoding the antigen-binding domain that binds the protein associated with the TCR complex comprises the nucleotide sequence of SEQ ID NO: 39 (huUCHT1).

In some embodiments, the antigen-binding domain that binds the protein associated with the TCR complex comprises an amino acid sequence having at least 70% sequence identity with the amino acid sequence of SEQ ID NO: 40 (huUCHT1). In some embodiments, the antigen-binding domain that binds the protein associated with the TCR complex comprises an amino acid sequence having at least 75% sequence identity with the amino acid sequence of SEQ ID NO: 40 (huUCHT1). In some embodiments, the antigen-binding domain that binds the protein associated with the TCR complex comprises an amino acid sequence having at least 80% sequence identity with the amino acid sequence of SEQ ID NO: 40 (huUCHT1). In some embodiments, the antigen-binding domain that binds the protein associated with the TCR complex comprises an amino acid sequence having at least 85% sequence identity with the amino acid sequence of SEQ ID NO: 40 (huUCHT1). In some embodiments, the antigen-binding domain that binds the protein associated with the TCR complex comprises an amino acid sequence having at least 90% sequence identity with the amino acid sequence of SEQ ID NO: 40 (huUCHT1). In some embodiments, the antigen-binding domain that binds the protein associated with the TCR complex comprises an amino acid sequence having at least 95% sequence identity with the amino acid sequence of SEQ ID NO: 40 (huUCHT1). In some embodiments, the antigen-binding domain that binds the protein associated with the TCR complex comprises an amino acid sequence having at least 96% sequence identity with the amino acid sequence of SEQ ID NO: 40 (huUCHT1). In some embodiments, the antigen-binding domain that binds the protein associated with the TCR complex comprises an amino acid sequence having at least 97% sequence identity with the amino acid sequence of SEQ ID NO: 40 (huUCHT1). In some embodiments, the antigen-binding domain that binds the protein associated with the TCR complex comprises an amino acid sequence having at least 98% sequence identity with the amino acid sequence of SEQ ID NO: 40 (huUCHT1). In some embodiments, the antigen-binding domain that binds the protein associated with the TCR complex comprises an amino acid sequence having at least 99% sequence identity with the amino acid sequence of SEQ ID NO: 40 (huUCHT1). In some embodiments, the antigen-binding domain that binds the protein associated with the TCR complex comprises the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 40 (huUCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 75% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 80% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 85% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 90% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 95% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 96% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 97% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 98% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 99% sequence identity with the nucleotide sequence of SEQ ID NO: 41 (huUCHT1 (Y177T)). In some embodiments, the polynucleotide encoding the antigen-binding domain that binds the protein associated with the TCR complex comprises the nucleotide sequence of SEQ ID NO: 41 (huUCHT1 (Y177T)).

In some embodiments, the antigen-binding domain that binds the protein associated with the TCR complex comprises an amino acid sequence having at least 70% sequence identity with the amino acid sequence of SEQ ID NO: 42 (huUCHT1 (Y177T)). In some embodiments, the antigen-binding domain that binds the protein associated with the TCR complex comprises an amino acid sequence having at least 75% sequence identity with the amino acid sequence of SEQ ID NO: 42 (huUCHT1 (Y177T)). In some embodiments, the antigen-binding domain that binds the protein associated with the TCR complex comprises an amino acid sequence having at least 80% sequence identity with the amino acid sequence of SEQ ID NO: 42 (huUCHT1 (Y177T)). In some embodiments, the antigen-binding domain that binds the protein associated with the TCR complex comprises an amino acid sequence having at least 85% sequence identity with the amino acid sequence of SEQ ID NO: 42 (huUCHT1 (Y177T)). In some embodiments, the antigen-binding domain that binds the protein associated with the TCR complex comprises an amino acid sequence having at least 90% sequence identity with the amino acid sequence of SEQ ID NO: 42 (huUCHT1 (Y177T)). In some embodiments, the antigen-binding domain that binds the protein associated with the TCR complex comprises an amino acid sequence having at least 95% sequence identity with the amino acid sequence of SEQ ID NO: 42 (huUCHT1 (Y177T)). In some embodiments, the antigen-binding domain that binds the protein associated with the TCR complex comprises an amino acid sequence having at least 96% sequence identity with the amino acid sequence of SEQ ID NO: 42 (huUCHT1 (Y177T)). In some embodiments, the antigen-binding domain that binds the protein associated with the TCR complex comprises an amino acid sequence having at least 97% sequence identity with the amino acid sequence of SEQ ID NO: 42 (huUCHT1 (Y177T)). In some embodiments, the antigen-binding domain that binds the protein associated with the TCR complex comprises an amino acid sequence having at least 98% sequence identity with the amino acid sequence of SEQ ID NO: 42 (huUCHT1 (Y177T)). In some embodiments, the antigen-binding domain that binds the protein associated with the TCR complex comprises an amino acid sequence having at least 99% sequence identity with the amino acid sequence of SEQ ID NO: 42 (huUCHT1 (Y177T)). In some embodiments, the antigen-binding domain that binds the protein associated with the TCR complex comprises the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 42 (huUCHT1 (Y177T)).

In some embodiments, the antigen-binding domain that binds to the protein associated with the TCR complex is OKT3. In some embodiments, the murine OKT3 antigen-binding domain is encoded by SEQ ID NO: 33. In some embodiments, the OKT3 antigen-binding domain comprises SEQ ID NO: 34.

In some embodiments, the polynucleotide encoding the antigen-binding domain that binds the protein associated with the TCR complex comprises a nucleotide sequence having at least 70% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 75% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 80% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 85% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 90% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 95% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 96% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 97% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 98% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 99% sequence identity with the nucleotide sequence of SEQ ID NO: 33 (OKT3). In some embodiments, the polynucleotide encoding the antigen-binding domain that binds the protein associated with the TCR complex comprises the nucleotide sequence of SEQ ID NO: 33 (OKT3).

In some embodiments, the antigen-binding domain that binds the protein associated with the TCR complex comprises an amino acid sequence having at least 70% sequence identity with the amino acid sequence of SEQ ID NO: 34 (OKT3). In some embodiments, the antigen-binding domain that binds the protein associated with the TCR complex comprises an amino acid sequence having at least 75% sequence identity with the amino acid sequence of SEQ ID NO: 34 (OKT3). In some embodiments, the antigen-binding domain that binds the protein associated with the TCR complex comprises an amino acid sequence having at least 80% sequence identity with the amino acid sequence of SEQ ID NO: 34 (OKT3). In some embodiments, the antigen-binding domain that binds the protein associated with the TCR complex comprises an amino acid sequence having at least 85% sequence identity with the amino acid sequence of SEQ ID NO: 34 (OKT3). In some embodiments, the antigen-binding domain that binds the protein associated with the TCR complex comprises an amino acid sequence having at least 90% sequence identity with the amino acid sequence of SEQ ID NO: 34 (OKT3). In some embodiments, the antigen-binding domain that binds the protein associated with the TCR complex comprises an amino acid sequence having at least 95% sequence identity with the amino acid sequence of SEQ ID NO: 34 (OKT3). In some embodiments, the antigen-binding domain that binds the protein associated with the TCR complex comprises an amino acid sequence having at least 96% sequence identity with the amino acid sequence of SEQ ID NO: 34 (OKT3). In some embodiments, the antigen-binding domain that binds the protein associated with the TCR complex comprises an amino acid sequence having at least 97% sequence identity with the amino acid sequence of SEQ ID NO: 34 (OKT3). In some embodiments, the antigen-binding domain that binds the protein associated with the TCR complex comprises an amino acid sequence having at least 98% sequence identity with the amino acid sequence of SEQ ID NO: 34 (OKT3). In some embodiments, the antigen-binding domain that binds the protein associated with the TCR complex comprises an amino acid sequence having at least 99% sequence identity with the amino acid sequence of SEQ ID NO: 34 (OKT3). In some embodiments, the antigen-binding domain that binds the protein associated with the TCR complex comprises the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 34 (OKT3).

In some embodiments, the antigen-binding domain that binds to the protein associated with the TCR complex is F6A. In some embodiments, the murine F6A antigen-binding domain is encoded by SEQ ID NO: 35. In some embodiments, the F6A antigen-binding domain comprises SEQ ID NO: 36.

In some embodiments, the polynucleotide encoding the antigen-binding domain that binds the protein associated with the TCR complex comprises a nucleotide sequence having at least 70% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 75% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 80% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 85% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 90% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 95% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 96% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 97% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 98% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 99% sequence identity with the nucleotide sequence of SEQ ID NO: 35 (F6A). In some embodiments, the polynucleotide encoding the antigen-binding domain that binds the protein associated with the TCR complex comprises the nucleotide sequence of SEQ ID NO: 35 (F6A).

In some embodiments, the antigen-binding domain that binds the protein associated with the TCR complex comprises an amino acid sequence having at least 70% sequence identity with the amino acid sequence of SEQ ID NO: 36 (F6A). In some embodiments, the antigen-binding domain that binds the protein associated with the TCR complex comprises an amino acid sequence having at least 75% sequence identity with the amino acid sequence of SEQ ID NO: 36 (F6A). In some embodiments, the antigen-binding domain that binds the protein associated with the TCR complex comprises an amino acid sequence having at least 80% sequence identity with the amino acid sequence of SEQ ID NO: 36 (F6A). In some embodiments, the antigen-binding domain that binds the protein associated with the TCR complex comprises an amino acid sequence having at least 85% sequence identity with the amino acid sequence of SEQ ID NO: 36 (F6A). In some embodiments, the antigen-binding domain that binds the protein associated with the TCR complex comprises an amino acid sequence having at least 90% sequence identity with the amino acid sequence of SEQ ID NO: 36 (F6A). In some embodiments, the antigen-binding domain that binds the protein associated with the TCR complex comprises an amino acid sequence having at least 95% sequence identity with the amino acid sequence of SEQ ID NO: 36 (F6A). In some embodiments, the antigen-binding domain that binds the protein associated with the TCR complex comprises an amino acid sequence having at least 96% sequence identity with the amino acid sequence of SEQ ID NO: 36 (F6A). In some embodiments, the antigen-binding domain that binds the protein associated with the TCR complex comprises an amino acid sequence having at least 97% sequence identity with the amino acid sequence of SEQ ID NO: 36 (F6A). In some embodiments, the antigen-binding domain that binds the protein associated with the TCR complex comprises an amino acid sequence having at least 98% sequence identity with the amino acid sequence of SEQ ID NO: 36 (F6A). In some embodiments, the antigen-binding domain that binds the protein associated with the TCR complex comprises an amino acid sequence having at least 99% sequence identity with the amino acid sequence of SEQ ID NO: 36 (F6A). In some embodiments, the antigen-binding domain that binds the protein associated with the TCR complex comprises the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 36 (F6A).

In some embodiments, the antigen-binding domain that binds to the protein associated with the TCR complex is L2K. In some embodiments, the murine L2K antigen-binding domain is encoded by SEQ ID NO: 37. In some embodiments, the L2K antigen-binding domain comprises SEQ ID NO: 38.

In some embodiments, the polynucleotide encoding the antigen-binding domain that binds the protein associated with the TCR complex comprises a nucleotide sequence having at least 70% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 75% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 80% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 85% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 90% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 95% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 96% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 97% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 98% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 99% sequence identity with the nucleotide sequence of SEQ ID NO: 37 (L2K). In some embodiments, the polynucleotide encoding the antigen-binding domain that binds the protein associated with the TCR complex comprises the nucleotide sequence of SEQ ID NO: 37 (L2K).

In some embodiments, the antigen-binding domain that binds the protein associated with the TCR complex comprises an amino acid sequence having at least 70% sequence identity with the amino acid sequence of SEQ ID NO: 38 (L2K). In some embodiments, the antigen-binding domain that binds the protein associated with the TCR complex comprises an amino acid sequence having at least 75% sequence identity with the amino acid sequence of SEQ ID NO: 38 (L2K). In some embodiments, the antigen-binding domain that binds the protein associated with the TCR complex comprises an amino acid sequence having at least 80% sequence identity with the amino acid sequence of SEQ ID NO: 38 (L2K). In some embodiments, the antigen-binding domain that binds the protein associated with the TCR complex comprises an amino acid sequence having at least 85% sequence identity with the amino acid sequence of SEQ ID NO: 38 (L2K). In some embodiments, the antigen-binding domain that binds the protein associated with the TCR complex comprises an amino acid sequence having at least 90% sequence identity with the amino acid sequence of SEQ ID NO: 38 (L2K). In some embodiments, the antigen-binding domain that binds the protein associated with the TCR complex comprises an amino acid sequence having at least 95% sequence identity with the amino acid sequence of SEQ ID NO: 38 (L2K). In some embodiments, the antigen-binding domain that binds the protein associated with the TCR complex comprises an amino acid sequence having at least 96% sequence identity with the amino acid sequence of SEQ ID NO: 38 (L2K). In some embodiments, the antigen-binding domain that binds the protein associated with the TCR complex comprises an amino acid sequence having at least 97% sequence identity with the amino acid sequence of SEQ ID NO: 38 (L2K). In some embodiments, the antigen-binding domain that binds the protein associated with the TCR complex comprises an amino acid sequence having at least 98% sequence identity with the amino acid sequence of SEQ ID NO: 38 (L2K). In some embodiments, the antigen-binding domain that binds the protein associated with the TCR complex comprises an amino acid sequence having at least 99% sequence identity with the amino acid sequence of SEQ ID NO: 38 (L2K). In some embodiments, the antigen-binding domain that binds the protein associated with the TCR complex comprises the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 38 (L2K).

Amino acid and nucleotide sequences of exemplary antigen-binding domains that bind a protein associated with the TCR complex are provided in Table 3.

TABLE 3
Table of Sequences

| SEQ ID NO | Description | Nucleotide/Amino Acid |
|---|---|---|
| SEQ ID NO: 31 | UCHT1¹ | Nucleotide |
| SEQ ID NO: 32 | UCHT1² | Amino Acid |
| SEQ ID NO: 33 | OKT3 | Nucleotide |
| SEQ ID NO: 34 | OKT3 | Amino Acid |
| SEQ ID NO: 35 | F6A | Nucleotide |
| SEQ ID NO: 36 | F6A | Amino Acid |
| SEQ ID NO: 37 | L2K | Nucleotide |
| SEQ ID NO: 38 | L2K | Amino Acid |
| SEQ ID NO: 39 | huUCHT1 | Nucleotide |
| SEQ ID NO: 40 | huUCHT1 | Amino Acid |
| SEQ ID NO: 41 | huUCHT1 (Y177T) | Nucleotide |
| SEQ ID NO: 42 | huUCHT1 (Y177T) | Amino Acid |
| SEQ ID NO: 43 | UCHT1 (Y182T) | Nucleotide |
| SEQ ID NO: 44 | UCHT1 (Y182T) | Amino Acid |

¹Light chain, nucleotides 1-324; Linker, nucleotides 325-387; Heavy chain, nucleotides 388-750
²Light chain, amino acids 1-108; Linker, amino acids 109-128; Heavy chain, amino acids 129-250

### Transmembrane Domain and Cytosolic Domain

In some embodiments, a GPC3 T cell antigen coupler polypeptide comprises a T cell receptor signaling domain polypeptide. In some embodiments, a GPC3 T cell antigen coupler polypeptide comprises a transmembrane domain of a TCR signaling domain. In some embodiments, a GPC3 T cell antigen coupler polypeptide comprises a cytosolic domain of a TCR signaling domain polypeptide. In some embodiments, a GPC3 T cell antigen coupler polypeptide comprises a transmembrane domain and a cytosolic domain of a TCR signaling domain polypeptide.

In some embodiments, the T cell receptor signaling domain polypeptide comprises a TCR co-receptor domain. In some embodiments, the TCR signaling domain polypeptide comprises a transmembrane domain and/or a cytosolic domain of a TCR co-receptor. In some embodiments, the TCR co-receptor is CD4, CD8, LAG3, or a chimeric variation thereof.

In some embodiments, the TCR co-receptor is CD4. In some embodiments, the GPC3 TAC comprises a transmembrane domain and a cytosolic domain of a CD4 co-receptor. In some embodiments, the polynucleotide encoding the cytosolic and transmembrane domain comprises a nucleotide sequence having at least 70% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 45 (CD4 transmembrane and cytosolic domain). In some embodiments, the polynucleotide encoding the cytosolic and transmembrane domain comprises the nucleotide sequence of SEQ ID NO: 45 (CD4 transmembrane and cytosolic domain). In some embodiments, the cytosolic and transmembrane domain comprise an amino acid sequence having at least 70% sequence identity with the amino acid sequence of SEQ ID NO: 46 (CD4 transmembrane and cytosolic domain). In some embodiments, the cytosolic and transmembrane domain comprise an amino acid sequence having at least 75% sequence identity with the amino acid sequence of SEQ ID NO: 46 (CD4 transmembrane and cytosolic domain). In some embodiments, the cytosolic and transmembrane domain comprise an amino acid sequence having at least 80% sequence identity with the amino acid sequence of SEQ ID NO: 46 (CD4 transmembrane and cytosolic domain). In some embodiments, the cytosolic and transmembrane domain comprise an amino acid sequence having at least 85% sequence identity with the amino acid sequence of SEQ ID NO: 46 (CD4 transmembrane and cytosolic domain). In some embodiments, the cytosolic and transmembrane domain comprise an amino acid sequence having at least 90% sequence identity with the amino acid sequence of SEQ ID NO: 46 (CD4 transmembrane and cytosolic domain). In some embodiments, the cytosolic and transmembrane domain comprise an amino acid sequence having at least 95% sequence identity with the amino acid sequence of SEQ ID NO: 46 (CD4 transmembrane and cytosolic domain). In some embodiments, the cytosolic and transmembrane domain comprise an amino acid sequence having at least 96% sequence identity with the amino acid sequence of SEQ ID NO: 46 (CD4 transmembrane and cytosolic domain). In some embodiments, the cytosolic and transmembrane domain comprise an amino acid sequence having at least 97% sequence identity with the amino acid sequence of SEQ ID NO: 46 (CD4 transmembrane and cytosolic domain). In some embodiments, the cytosolic and transmembrane domain comprise an amino acid sequence having at least 98% sequence identity with the amino acid sequence of SEQ ID NO: 46 (CD4 transmembrane and cytosolic domain). In some embodiments, the cytosolic and transmembrane domain comprise an amino acid sequence having at least 99% sequence identity with the amino acid sequence of SEQ ID NO: 46 (CD4 transmembrane and cytosolic domain). In some embodiments, the cytosolic and transmembrane domain comprise the amino acid sequence of SEQ ID NO: 46 (CD4 transmembrane and cytosolic domain).

In some embodiments, the TCR co-receptor is CD8. In some embodiments, the TCR co-receptor is CD8α. In some embodiments, the polynucleotide encoding the cytosolic and transmembrane domain comprises a nucleotide sequence having at least 70% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 47 (CD8 transmembrane and cytosolic domain). In some embodiments, the polynucleotide encoding the cytosolic and transmembrane domain comprises the nucleotide sequence of SEQ ID NO: 47 (CD8 transmembrane and cytosolic domain). In some embodiments, the cytosolic and transmembrane domain comprise an amino acid sequence having at least 70% sequence identity with the amino acid sequence of SEQ ID NO: 48 (CD8 transmembrane and cytosolic domain). In some embodiments, the cytosolic and transmembrane domain comprise an amino acid sequence having at least 75% sequence identity with the amino acid sequence of SEQ ID NO: 48 (CD8 transmembrane and cytosolic domain). In some embodiments, the cytosolic and transmembrane domain comprise an amino acid sequence having at least 80% sequence identity with the amino acid sequence of SEQ ID NO: 48 (CD8 transmembrane and cytosolic domain). In some embodiments, the cytosolic and transmembrane domain comprise an amino acid sequence having at least 85% sequence identity with the amino acid sequence of SEQ ID NO: 48 (CD8 transmembrane and cytosolic domain). In some embodiments, the cytosolic and transmembrane domain comprise an amino acid sequence having at least 90% sequence identity with the amino acid sequence of SEQ ID NO: 48 (CD8 transmembrane and cytosolic domain). In some embodiments, the cytosolic and transmembrane domain comprise an amino acid sequence having at least 95% sequence identity with the amino acid sequence of SEQ ID NO: 48 (CD8 transmembrane and cytosolic domain). In some embodiments, the cytosolic and transmembrane domain comprise an amino acid sequence having at least 96% sequence identity with the amino acid sequence of SEQ ID NO: 48 (CD8 transmembrane and cytosolic domain). In some embodiments, the cytosolic and transmembrane domain comprise an amino acid sequence having at least 97% sequence identity with the amino acid sequence of SEQ ID NO: 48 (CD8 transmembrane and cytosolic domain). In some embodiments, the cytosolic and transmembrane domain comprise an amino acid sequence having at least 98% sequence identity with the amino acid sequence of SEQ ID NO: 48 (CD8 transmembrane and cytosolic domain). In some embodiments, the cytosolic and transmembrane domain comprise an amino acid sequence having at least 99% sequence identity with the amino acid sequence of SEQ ID NO: 48 (CD8 transmembrane and cytosolic domain). In some embodiments, the cytosolic and transmembrane domain comprise the amino acid sequence of SEQ ID NO: 48 (CD8 transmembrane and cytosolic domain).

In some embodiments, the TCR signaling domain polypeptide comprises a chimera of sequences or domains from co-receptors. In some embodiments, the TCR signaling domain polypeptide comprises a chimera of CD8α and CD8β, wherein the CD8α arginine rich region is replaced with the CD8β arginine rich region (CD8α+R(β) chimera). In some embodiments, the polynucleotide encoding the cytosolic and transmembrane domain comprises a nucleotide sequence having at least 70% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 75% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 80% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 85% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 90% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 95% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 96% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 97% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 98% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 99% sequence identity with the nucleotide sequence of SEQ ID NO: 49 (CD8α+R(β) chimera). In some embodiments, the polynucleotide encoding the cytosolic and transmembrane domain comprises the nucleotide sequence of SEQ ID NO: 49 (CD8α+R(β) chimera). In some embodiments, the cytosolic and transmembrane domain comprise an amino acid sequence having at least 70% sequence identity with the amino acid sequence of SEQ ID NO: 50 (CD8α+R(β) chimera). In some embodiments, the cytosolic and transmembrane domain comprise an amino acid sequence having at least 75% sequence identity with the amino acid sequence of SEQ ID NO: 50 (CD8α+R(β) chimera). In some embodiments, the cytosolic and transmembrane domain comprise an amino acid sequence having at least 80% sequence identity with the amino acid sequence of SEQ ID NO: 50 (CD8α+R(β) chimera). In some embodiments, the cytosolic and transmembrane domain comprise an amino acid sequence having at least 85% sequence identity with the amino acid sequence of SEQ ID NO: 50 (CD8α+R(β) chimera). In some embodiments, the cytosolic and transmembrane domain comprise an amino acid sequence having at least 90% sequence identity with the amino acid sequence of SEQ ID NO: 50 (CD8α+R(β) chimera). In some embodiments, the cytosolic and transmembrane domain comprise an amino acid sequence having at least 95% sequence identity with the amino acid sequence of SEQ ID NO: 50 (CD8α+R(β) chimera). In some embodiments, the cytosolic and transmembrane domain comprise an amino acid sequence having at least 96% sequence identity with the amino acid sequence of SEQ ID NO: 50 (CD8α+R(β) chimera). In some embodiments, the cytosolic and transmembrane domain comprise an amino acid sequence having at least 97% sequence identity with the amino acid sequence of SEQ ID NO: 50 (CD8α+R(β) chimera). In some embodiments, the cytosolic and transmembrane domain comprise an amino acid sequence having at least 98% sequence identity with the amino acid sequence of SEQ ID NO: 50 (CD8α+R(β) chimera). In some embodiments, the cytosolic and transmembrane domain comprise an amino acid sequence having at least 99% sequence identity with the amino acid sequence of SEQ ID NO: 50 (CD8α+R(β) chimera). In some embodiments, the cytosolic and transmembrane domain comprise the amino acid sequence of SEQ ID NO: 50 (CD8α+R(β) chimera).

In some embodiments, the TCR signaling domain polypeptide comprises a chimera of CD8α and CD8β, where the CD8α CXCP domain, which contains an Lck binding motif, is appended to the C-terminus of the CD8β cytosolic domain (CD8β+Lck chimera). In some embodiments, the polynucleotide encoding the cytosolic and transmembrane domain comprises a nucleotide sequence having at least 70% sequence identity with the nucleotide sequence of SEQ ID NO: 51 (CD8β+Lck chimera). In some embodiments, the polynucleotide encoding the cytosolic and transmembrane domain comprises a nucleotide sequence having at least 75% sequence identity with the nucleotide sequence of SEQ ID NO: 51 (CD8β+Lck chimera). In some embodiments, the polynucleotide encoding the cytosolic and transmembrane domain comprises a nucleotide sequence having at least 80% sequence identity with the nucleotide sequence of SEQ ID NO: 51 (CD8β+Lck chimera). In some embodiments, the polynucleotide encoding the cytosolic and transmembrane domain comprises a nucleotide sequence having at least 85% sequence identity with the nucleotide sequence of SEQ ID NO: 51 (CD8β+Lck chimera). In some embodiments, the polynucleotide encoding the cytosolic and transmembrane domain comprises a nucleotide sequence having at least 90% sequence identity with the nucleotide sequence of SEQ ID NO: 51 (CD8β+Lck chimera). In some embodiments, the polynucleotide encoding the cytosolic and transmembrane domain comprises a nucleotide sequence having at least 95% sequence identity with the nucleotide sequence of SEQ ID NO: 51 (CD8β+Lck chimera). In some embodiments, the polynucleotide encoding the cytosolic and transmembrane domain comprises a nucleotide sequence having at least 96% sequence identity with the nucleotide sequence of SEQ ID NO: 51 (CD8β+Lck chimera). In some embodiments, the polynucleotide encoding the cytosolic and transmembrane domain comprises a nucleotide sequence having at least 97% sequence identity with the nucleotide sequence of SEQ ID NO: 51 (CD8β+Lck chimera). In some embodiments, the polynucleotide encoding the cytosolic and transmembrane domain comprises a nucleotide sequence having at least 98% sequence identity with the nucleotide sequence of SEQ ID NO: 51 (CD8β+Lck chimera). In some embodiments, the polynucleotide encoding the cytosolic and transmembrane domain comprises a nucleotide sequence having at least 99% sequence identity with the nucleotide sequence of SEQ ID NO: 51 (CD8β+Lck chimera). In some embodiments, the polynucleotide encoding the cytosolic and transmembrane domain comprises the nucleotide sequence of SEQ ID NO: 51 (CD8β+Lck chimera). In some embodiments, the cytosolic and transmembrane domain comprise an amino acid sequence having at least 70% sequence identity with the amino acid sequence of SEQ ID NO: 52 (CD8β+Lck chimera). In some embodiments, the cytosolic and transmembrane domain comprise an amino acid sequence having at least 75% sequence identity with the amino acid sequence of SEQ ID NO: 52 (CD8β+Lck chimera). In some embodiments, the cytosolic and transmembrane domain comprise an amino acid sequence having at least 80% sequence identity with the amino acid sequence of SEQ ID NO: 52 (CD8β+Lck chimera). In some embodiments, the cytosolic and transmembrane domain comprise an amino acid sequence having at least 85% sequence identity with the amino acid sequence of SEQ ID NO: 52 (CD8β+Lck chimera). In some embodiments, the cytosolic and transmembrane domain comprise an amino acid sequence having at least 90% sequence identity with the amino acid sequence of SEQ ID NO: 52 (CD8β+Lck chimera). In some embodiments, the cytosolic and transmembrane domain comprise an amino acid sequence having at least 95% sequence identity with the amino acid sequence of SEQ ID NO: 52 (CD8β+Lck chimera). In some embodiments, the cytosolic and transmembrane domain comprise an amino acid sequence having at least 96% sequence identity with the amino acid sequence of SEQ ID NO: 52 (CD8β+Lck chimera). In some embodiments, the cytosolic and transmembrane domain comprise an amino acid sequence having at least 97% sequence identity with the amino acid sequence of SEQ ID NO: 52 (CD8β+Lck chimera). In some embodiments, the cytosolic and transmembrane domain comprise an amino acid sequence having at least 98% sequence identity with the amino acid sequence of SEQ ID NO: 52 (CD8β+Lck chimera). In some embodiments, the cytosolic and transmembrane domain comprise an amino acid sequence having at least 99% sequence identity with the amino acid sequence of SEQ ID NO: 52 (CD8β+Lck chimera). In some embodiments, the cytosolic and transmembrane domain comprise the amino acid sequence of SEQ ID NO: 52 (CD8β+Lck chimera).

In some embodiments, the TCR signaling domain polypeptide includes both a cytosolic domain and a transmembrane domain of a TCR co-receptor protein. In some embodiments, the cytosolic domain and transmembrane domain are from the same co-receptor or from different co-receptors.

Amino acid and nucleotide sequences of exemplary transmembrane and cytosolic domains are provided in Table 4.

TABLE 4
Table of Sequences

| SEQ ID NO | Description | Nucleotide/Amino Acid |
|---|---|---|
| SEQ ID NO: 45 | CD4 Domain¹ | Nucleotide |
| SEQ ID NO: 46 | CD4 Domain² | Amino Acid |
| SEQ ID NO: 47 | CD8α Domain | Nucleotide |
| SEQ ID NO: 48 | CD8α Domain | Amino Acid |
| SEQ ID NO: 49 | CD8α + R(β) Domain | Nucleotide |
| SEQ ID NO: 50 | CD8α + R(β) Domain | Amino Acid |
| SEQ ID NO: 51 | CD8 α + Lck Domain | Nucleotide |
| SEQ ID NO: 52 | CD8 α + Lck Domain | Amino Acid |

¹Extracellular linker, nucleotides 1-66; Transmembrane domain, nucleotides 67-132; Cytosolic domain, nucleotides 133-254
²Extracellular linker, amino acids 1-22; Transmembrane domain, amino acids 23-44; Cytosolic domain, amino acids 45-84

### Configurations, Linkers, and Connectors

In some embodiments, a nucleic acid disclosed herein is in an order of (1) a first polynucleotide encoding an antigen-binding domain that binds GPC3; (2) a second polynucleotide encoding an antigen-binding domain that binds a TCR complex; (3) a third polynucleotide encoding a transmembrane domain and a cytosolic domain. In some embodiments, a nucleic acid disclosed herein is in an order of (1) a first polynucleotide encoding an antigen-binding domain that binds GPC3; (2) a second polynucleotide encoding an antigen-binding domain that binds a TCR complex; (3) a third polynucleotide encoding a transmembrane domain and a cytosolic domain, wherein the order is 5′ end to 3′ end. In some embodiments, a nucleic acid disclosed herein is in an order of (1) a first polynucleotide encoding an antigen-binding domain that binds GPC3; (2) a second polynucleotide encoding an antigen-binding domain that binds a TCR complex; (3) a third polynucleotide encoding a transmembrane domain and a cytosolic domain, wherein the order is 3′ end to 5′ end. In some embodiments, a nucleic acid described herein is in an order of (1) a first polynucleotide encoding an antigen-binding domain that binds a TCR complex; (2) a second polynucleotide encoding an antigen-binding domain that binds GPC3; (3) a third polynucleotide encoding a transmembrane domain and a cytosolic domain. In some embodiments, a nucleic acid described herein is in an order of (1) a first polynucleotide encoding an antigen-binding domain that binds a TCR complex; (2) a second polynucleotide encoding an antigen-binding domain that binds GPC3; (3) a third polynucleotide encoding a transmembrane domain and a cytosolic domain, wherein the order is 5′ end to 3′ end. In some embodiments, a nucleic acid described herein is in an order of (1) a first polynucleotide encoding an antigen-binding domain that binds a TCR complex; (2) a second polynucleotide encoding an antigen-binding domain that binds GPC3; (3) a third polynucleotide encoding a transmembrane domain and a cytosolic domain, wherein the order is 3′ end to 5′ end.

In some embodiments, a GPC3 TAC polypeptide disclosed herein is in an order of (1) an antigen-binding domain that binds GPC3; (2) an antigen-binding domain that binds a TCR complex; (3) a transmembrane domain and a cytosolic domain, wherein the order is N-terminus to C-terminus. In some embodiments, a GPC3 TAC polypeptide disclosed herein is in an order of (1) an antigen-binding domain that binds GPC3; (2) an antigen-binding domain that binds a TCR complex; (3) a transmembrane domain and a cytosolic domain, wherein the order is C-terminus to N-terminus. In some embodiments, a GPC3 TAC polypeptide described herein is in an order of (1) an antigen-binding domain that binds a TCR complex; (2) an antigen-binding domain that binds GPC3; (3) a transmembrane domain and a cytosolic domain, wherein the order is N-terminus to C-terminus. In some embodiments, a GPC3 TAC polypeptide described herein is in an order of (1) an antigen-binding domain that binds a TCR complex; (2) an antigen-binding domain that binds GPC3; (3) a transmembrane domain and a cytosolic domain, wherein the order is C-terminus to N-terminus.

In some embodiments, the antigen-binding domain that binds GPC3, the antigen-binding domain that binds the TCR complex, and/or the transmembrane domain and cytosolic domain are directly fused. For example, the antigen-binding domain that binds GPC3 and the transmembrane domain and cytosolic domain are both fused to the antigen-binding domain that binds the TCR complex. In some embodiments, the antigen-binding domain that binds GPC3, the antigen-binding domain that binds the TCR complex, and/or the transmembrane domain and cytosolic domain are joined by at least one linker. In some embodiments, the antigen-binding domain that binds GPC3 and the antigen-binding domain that binds the TCR complex are directly fused, and joined to the transmembrane domain and cytosolic domain by a linker. In some embodiments, the antigen-binding domain that binds the TCR complex and the transmembrane domain and cytosolic domain are directly fused, and joined to the antigen-binding domain that binds GPC3 by a linker.

In some embodiments, the linker is a peptide linker. In some embodiments, the peptide linker comprises 1 to 40 amino acids. In some embodiments, the peptide linker comprises 1 to 30 amino acids. In some embodiments, the peptide linker comprises 1 to 15 amino acids. In some embodiments, the peptide linker comprises 1 to 10 amino acids. In some embodiments, the peptide linker comprises 1 to 6 amino acids. In some embodiments, the peptide linker comprises 30 to 40 amino acids. In some embodiments, the peptide linker comprises 32 to 36 amino acids. In some embodiments, the peptide linker comprises 5 to 30 amino acids. In some embodiments, the peptide linker comprises 5 amino acids. In some embodiments, the peptide linker comprises 10 amino acids. In some embodiments, the peptide linker comprises 15 amino acids. In some embodiments, the peptide linker comprises 20 amino acids. In some embodiments, the peptide linker comprises 25 amino acids. In some embodiments, the peptide linker comprises 30 amino acids. In some embodiments, the peptide linker comprises a glycine and/or serine-rich linker.

In some embodiments, the at least one linker comprises an amino acid sequence having at least 80% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 85% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 90% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 95% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 96% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 97% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 98% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 99% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker).

In some embodiments, the peptide linker that joins the antigen-binding domain that binds GPC3 to the antigen-binding domain that binds a TCR complex (e.g., UCHT1) is known as the connector to distinguish this protein domain from other linkers in the TAC. The connector may be of any size. In some embodiments, the connector between the antigen-binding domain that binds a TCR complex and the antigen-binding domain that binds GPC3 is a short helix comprising SEQ ID NO: 12. In some embodiments, the connector between the antigen-binding domain that binds a TCR complex and the antigen-binding domain that binds GPC3 is a short helix encoded by SEQ ID NO: 11. In some embodiments, the connector between the antigen-binding domain that binds a TCR complex and the antigen-binding domain that binds GPC3 is a long helix comprising SEQ ID NO: 14. In some embodiments, the connector between the antigen-binding domain that binds a TCR complex and the antigen-binding domain that binds GPC3 is a long helix encoded by SEQ ID NO: 13. In some embodiments, the connector between the antigen-binding domain that binds a TCR complex and the antigen-binding domain that binds GPC3 is a large domain comprising SEQ ID NO: 16. In some embodiments, the connector between the antigen-binding domain that binds a TCR complex and the antigen-binding domain that binds GPC3 is a large domain encoded by SEQ ID NO: 15.

In some embodiments, a nucleic acid or TAC disclosed herein comprises a leader sequence. In some embodiments, the leader sequence is encoded by a nucleotide sequence having at least 80% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 85% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 90% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 95% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 96% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 97% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 98% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence is encoded by a nucleotide sequence having at least 99% sequence identity with the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader). In some embodiments, the leader sequence comprises the nucleotide sequence of SEQ ID NO: 1 (muIgG leader), SEQ ID NO: 17 (huIgG leader), SEQ ID NO: 19 (huCD8a leader), or SEQ ID NO: 29 (huCD8a leader).

In some embodiments, a nucleic acid or TAC disclosed herein comprises a leader sequence. In some embodiments, the leader sequence comprises an amino acid sequence having at least 80% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 85% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 90% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 95% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 96% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 97% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 98% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises an amino acid sequence having at least 99% sequence identity with the amino acid sequence of SEQ ID NO: 2 (muIgG leader), SEQ ID NO: 18 (huIgG leader), or SEQ ID NO: 20 (huCD8a leader). In some embodiments, the leader sequence comprises the amino acid sequence of SEQ ID NO: 2 (muIgG leader), SEQ ID NO: 18 (huIgG leader), or SEQ ID NO: 20 (huCD8a leader).

In some embodiments, a GPC3 T cell antigen coupler polypeptide comprises a tag, e.g., a Myc tag. In some embodiments, the tag comprises an amino acid sequence having at least 80% identity with the amino acid sequence of SEQ ID NO: 4 (Myc Tag). In some embodiments, the tag comprises an amino acid sequence having at least 85% identity with the amino acid sequence of SEQ ID NO: 4 (Myc Tag). In some embodiments, the tag comprises an amino acid sequence having at least 90% identity with the amino acid sequence of SEQ ID NO: 4 (Myc Tag). In some embodiments, the tag comprises an amino acid sequence having at least 95% identity with the amino acid sequence of SEQ ID NO: 4 (Myc Tag). In some embodiments, the tag comprises an amino acid sequence having at least 96% identity with the amino acid sequence of SEQ ID NO: 4 (Myc Tag). In some embodiments, the tag comprises an amino acid sequence having at least 97% identity with the amino acid sequence of SEQ ID NO: 4 (Myc Tag). In some embodiments, the tag comprises an amino acid sequence having at least 98% identity with the amino acid sequence of SEQ ID NO: 4 (Myc Tag). In some embodiments, the tag comprises an amino acid sequence having at least 99% identity with the amino acid sequence of SEQ ID NO: 4 (Myc Tag). In some embodiments, the tag comprises the amino acid sequence of SEQ ID NO: 4 (Myc Tag).

Amino acid and nucleotide sequences of exemplary linkers, connectors, tags, and leader sequences are provided in Table 5.

TABLE 5
Table of Sequences

| | | Nucleotide/Amino |
|---|---|---|
| SEQ ID NO | Description | Acid |
| SEQ ID NO: 1 | muIgG leader | Nucleotide |
| | (secretion signal) | |
| SEQ ID NO: 2 | muIgG leader | Amino Acid |
| | (secretion signal) | |
| SEQ ID NO: 3 | Myc Tag | Nucleotide |
| SEQ ID NO: 4 | Myc Tag | Amino Acid |
| SEQ ID NO: 5 | Linker 1 | Nucleotide |
| SEQ ID NO: 6 | Linker 1 | Amino Acid |
| SEQ ID NO: 7 | Linker 2 | Nucleotide |
| SEQ ID NO: 8 | Linker 2 | Amino Acid |
| SEQ ID NO: 9 | CD4 linker | Nucleotide |
| SEQ ID NO: 10 | CD4 linker | Amino Acid |
| SEQ ID NO: 11 | Short Helix connector | Nucleotide |
| SEQ ID NO: 12 | Short Helix connector | Amino Acid |
| SEQ ID NO: 13 | Long Helix connector | Nucleotide |
| SEQ ID NO: 14 | Long Helix connector | Amino Acid |
| SEQ ID NO: 15 | Large domain connector | Nucleotide |
| SEQ ID NO: 16 | Large domain connector | Amino Acid |
| SEQ ID NO: 17 | huIgG | Nucleotide |
| SEQ ID NO: 18 | huIgG | Amino Acid |
| SEQ ID NO: 19 | huCD8a -1 | Nucleotide |
| SEQ ID NO: 20 | huCD8a -1 | Amino Acid |
| SEQ ID NO: 21 | Whitlow Linker | Nucleotide |
| SEQ ID NO: 22 | Whitlow Linker | Amino Acid |
| SEQ ID NO: 23 | (G4S)3 linker | Nucleotide |
| SEQ ID NO: 24 | (G4S)3 linker | Amino Acid |
| SEQ ID NO: 25 | (G4S)4 linker | Nucleotide |
| SEQ ID NO: 26 | (G4S)4 linker | Amino Acid |
| SEQ ID NO: 27 | G4S linker | Nucleotide |
| SEQ ID NO: 28 | G4S linker | Amino Acid |
| SEQ ID NO: 29 | huCD8a -2 | Nucleotide |
| SEQ ID NO: 30 | huCD8a -2 | Amino Acid |

### Specific TACs

Disclosed herein, in certain embodiments, are GPC3-TAC proteins comprising an amino acid sequence of any one of SEQ ID NOs: 54, 56, 58, 60, 62, 64, 66, 68, 70, 72, 74, or 76.

In some embodiments, the GPC3-TAC protein comprises an amino acid sequence having at least 80% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 85% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 90% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 95% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 96% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 97% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 98% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence having at least 99% sequence identity with the amino acid sequence of any one of SEQ ID NOs: 54, 56, 58, 60, 62, 64, 66, 68, 70, 72, 74, or 76. In some embodiments, the GPC3-TAC protein comprises an amino acid sequence of any one of SEQ ID NOs: 54, 56, 58, 60, 62, 64, 66, 68, 70, 72, 74, or 76.

In some embodiments, the GPC3-TAC protein comprises an amino acid sequence having at least 80% sequence identity with the amino acid sequence of SEQ ID NO: 54. In some embodiments, the GPC3-TAC protein comprises an amino acid sequence having at least 85% sequence identity with the amino acid sequence of SEQ ID NO: 54. In some embodiments, the GPC3-TAC protein comprises an amino acid sequence having at least 90% sequence identity with the amino acid sequence of SEQ ID NO: 54. In some embodiments, the GPC3-TAC protein comprises an amino acid sequence having at least 95% sequence identity with the amino acid sequence of SEQ ID NO: 54. In some embodiments, the GPC3-TAC protein comprises an amino acid sequence having at least 96% sequence identity with the amino acid sequence of SEQ ID NO: 54. In some embodiments, the GPC3-TAC protein comprises an amino acid sequence having at least 97% sequence identity with the amino acid sequence of SEQ ID NO: 54. In some embodiments, the GPC3-TAC protein comprises an amino acid sequence having at least 98% sequence identity with the amino acid sequence of SEQ ID NO: 54. In some embodiments, the GPC3-TAC protein comprises an amino acid sequence having at least 99% sequence identity with the amino acid sequence of SEQ ID NO: 54. In some embodiments, the GPC3-TAC protein comprises an amino acid sequence of SEQ ID NO: 54.

In some embodiments, the GPC3-TAC protein comprises an amino acid sequence having at least 80% sequence identity with the amino acid sequence of SEQ ID NO: 56. In some embodiments, the GPC3-TAC protein comprises an amino acid sequence having at least 85% sequence identity with the amino acid sequence of SEQ ID NO: 56. In some embodiments, the GPC3-TAC protein comprises an amino acid sequence having at least 90% sequence identity with the amino acid sequence of SEQ ID NO: 56. In some embodiments, the GPC3-TAC protein comprises an amino acid sequence having at least 95% sequence identity with the amino acid sequence of SEQ ID NO: 56. In some embodiments, the GPC3-TAC protein comprises an amino acid sequence having at least 96% sequence identity with the amino acid sequence of SEQ ID NO: 56. In some embodiments, the GPC3-TAC protein comprises an amino acid sequence having at least 97% sequence identity with the amino acid sequence of SEQ ID NO: 56. In some embodiments, the GPC3-TAC protein comprises an amino acid sequence having at least 98% sequence identity with the amino acid sequence of SEQ ID NO: 56. In some embodiments, the GPC3-TAC protein comprises an amino acid sequence having at least 99% sequence identity with the amino acid sequence of SEQ ID NO: 56. In some embodiments, the GPC3-TAC protein comprises an amino acid sequence of SEQ ID NO: 56.

In some embodiments, the GPC3-TAC protein comprises an amino acid sequence having at least 80% sequence identity with the amino acid sequence of SEQ ID NO: 58. In some embodiments, the GPC3-TAC protein comprises an amino acid sequence having at least 85% sequence identity with the amino acid sequence of SEQ ID NO: 58. In some embodiments, the GPC3-TAC protein comprises an amino acid sequence having at least 90% sequence identity with the amino acid sequence of SEQ ID NO: 58. In some embodiments, the GPC3-TAC protein comprises an amino acid sequence having at least 95% sequence identity with the amino acid sequence of SEQ ID NO: 58. In some embodiments, the GPC3-TAC protein comprises an amino acid sequence having at least 96% sequence identity with the amino acid sequence of SEQ ID NO: 58. In some embodiments, the GPC3-TAC protein comprises an amino acid sequence having at least 97% sequence identity with the amino acid sequence of SEQ ID NO: 58. In some embodiments, the GPC3-TAC protein comprises an amino acid sequence having at least 98% sequence identity with the amino acid sequence of SEQ ID NO: 58. In some embodiments, the GPC3-TAC protein comprises an amino acid sequence having at least 99% sequence identity with the amino acid sequence of SEQ ID NO: 58. In some embodiments, the GPC3-TAC protein comprises an amino acid sequence of SEQ ID NO: 58.

In some embodiments, the GPC3-TAC protein comprises an amino acid sequence having at least 80% sequence identity with the amino acid sequence of SEQ ID NO: 60. In some embodiments, the GPC3-TAC protein comprises an amino acid sequence having at least 85% sequence identity with the amino acid sequence of SEQ ID NO: 60. In some embodiments, the GPC3-TAC protein comprises an amino acid sequence having at least 90% sequence identity with the amino acid sequence of SEQ ID NO: 60. In some embodiments, the GPC3-TAC protein comprises an amino acid sequence having at least 95% sequence identity with the amino acid sequence of SEQ ID NO: 60. In some embodiments, the GPC3-TAC protein comprises an amino acid sequence having at least 96% sequence identity with the amino acid sequence of SEQ ID NO: 60. In some embodiments, the GPC3-TAC protein comprises an amino acid sequence having at least 97% sequence identity with the amino acid sequence of SEQ ID NO: 60. In some embodiments, the GPC3-TAC protein comprises an amino acid sequence having at least 98% sequence identity with the amino acid sequence of SEQ ID NO: 60. In some embodiments, the GPC3-TAC protein comprises an amino acid sequence having at least 99% sequence identity with the amino acid sequence of SEQ ID NO: 60. In some embodiments, the GPC3-TAC protein comprises an amino acid sequence of SEQ ID NO: 60.

In some embodiments, the GPC3-TAC protein comprises an amino acid sequence having at least 80% sequence identity with the amino acid sequence of SEQ ID NO: 62. In some embodiments, the GPC3-TAC protein comprises an amino acid sequence having at least 85% sequence identity with the amino acid sequence of SEQ ID NO: 62. In some embodiments, the GPC3-TAC protein comprises an amino acid sequence having at least 90% sequence identity with the amino acid sequence of SEQ ID NO: 62. In some embodiments, the GPC3-TAC protein comprises an amino acid sequence having at least 95% sequence identity with the amino acid sequence of SEQ ID NO: 62. In some embodiments, the GPC3-TAC protein comprises an amino acid sequence having at least 96% sequence identity with the amino acid sequence of SEQ ID NO: 62. In some embodiments, the GPC3-TAC protein comprises an amino acid sequence having at least 97% sequence identity with the amino acid sequence of SEQ ID NO: 62. In some embodiments, the GPC3-TAC protein comprises an amino acid sequence having at least 98% sequence identity with the amino acid sequence of SEQ ID NO: 62. In some embodiments, the GPC3-TAC protein comprises an amino acid sequence having at least 99% sequence identity with the amino acid sequence of SEQ ID NO: 62. In some embodiments, the GPC3-TAC protein comprises an amino acid sequence of SEQ ID NO: 62.

In some embodiments, the GPC3-TAC protein comprises an amino acid sequence having at least 80% sequence identity with the amino acid sequence of SEQ ID NO: 64. In some embodiments, the GPC3-TAC protein comprises an amino acid sequence having at least 85% sequence identity with the amino acid sequence of SEQ ID NO: 64. In some embodiments, the GPC3-TAC protein comprises an amino acid sequence having at least 90% sequence identity with the amino acid sequence of SEQ ID NO: 64. In some embodiments, the GPC3-TAC protein comprises an amino acid sequence having at least 95% sequence identity with the amino acid sequence of SEQ ID NO: 64. In some embodiments, the GPC3-TAC protein comprises an amino acid sequence having at least 96% sequence identity with the amino acid sequence of SEQ ID NO: 64. In some embodiments, the GPC3-TAC protein comprises an amino acid sequence having at least 97% sequence identity with the amino acid sequence of SEQ ID NO: 64. In some embodiments, the GPC3-TAC protein comprises an amino acid sequence having at least 98% sequence identity with the amino acid sequence of SEQ ID NO: 64. In some embodiments, the GPC3-TAC protein comprises an amino acid sequence having at least 99% sequence identity with the amino acid sequence of SEQ ID NO: 64. In some embodiments, the GPC3-TAC protein comprises an amino acid sequence of SEQ ID NO: 64.

In some embodiments, the GPC3-TAC protein comprises an amino acid sequence having at least 80% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of any one of SEQ ID NOs: 54, 56, 58, 60, 62, 64, 66, 68, 70, 72, 74, or 76, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 54, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 54, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 56, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 56, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 58, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 58, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 60, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 60, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 62, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 62, wherein the CDR sequences of the GPC3-TAC protein sequence have 100% sequence identity to the CDR sequences of the sequence of the SEQ ID NO.

In some embodiments, the GPC3-TAC protein comprises an amino acid sequence having at least 80% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 85% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 90% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 95% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 96% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 97% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 98% sequence identity with the amino acid sequence of SEQ ID NO: 64, wherein the CDR sequences of the GPC3-TAC protein sequence have 100% sequence identity to the CDR sequences of the sequence of the SEQ ID NO. In some embodiments, the GPC3-TAC protein comprises an amino acid sequence having at least 99% sequence identity with the amino acid sequence of SEQ ID NO: 64, wherein the CDR sequences of the GPC3-TAC protein sequence have 100% sequence identity to the CDR sequences of the sequence of the SEQ ID NO.

In some embodiments, the GPC3-TAC protein is encoded by a nucleic acid sequence having at least 80% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 85% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 90% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 95% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 96% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 97% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 98% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence having at least 99% sequence identity with the nucleic acid sequence of any one of SEQ ID NOs: 53, 55, 57, 59, 61, 63, 65, 67, 69, 71, 73, or 75. In some embodiments, the GPC3-TAC protein is encoded by a nucleic acid sequence of any one of SEQ ID NOs: 53, 55, 57, 59, 61, 63, 65, 67, 69, 71, 73, or 75.

In some embodiments, the GPC3-TAC protein is encoded by a nucleic acid sequence having at least 80% sequence identity with the nucleic acid sequence of SEQ ID NO: 53. In some embodiments, the GPC3-TAC protein is encoded by a nucleic acid sequence having at least 85% sequence identity with the nucleic acid sequence of SEQ ID NO: 53. In some embodiments, the GPC3-TAC protein is encoded by a nucleic acid sequence having at least 90% sequence identity with the nucleic acid sequence of SEQ ID NO: 53. In some embodiments, the GPC3-TAC protein is encoded by a nucleic acid sequence having at least 95% sequence identity with the nucleic acid sequence of SEQ ID NO: 53. In some embodiments, the GPC3-TAC protein is encoded by a nucleic acid sequence having at least 96% sequence identity with the nucleic acid sequence of SEQ ID NO: 53. In some embodiments, the GPC3-TAC protein is encoded by a nucleic acid sequence having at least 97% sequence identity with the nucleic acid sequence of SEQ ID NO: 53. In some embodiments, the GPC3-TAC protein is encoded by a nucleic acid sequence having at least 98% sequence identity with the nucleic acid sequence of SEQ ID NO: 53. In some embodiments, the GPC3-TAC protein is encoded by a nucleic acid sequence having at least 99% sequence identity with the nucleic acid sequence of SEQ ID NO: 53. In some embodiments, the GPC3-TAC protein is encoded by a nucleic acid sequence of SEQ ID NO: 53.

In some embodiments, the GPC3-TAC protein is encoded by a nucleic acid sequence having at least 80% sequence identity with the nucleic acid sequence of SEQ ID NO: 55. In some embodiments, the GPC3-TAC protein is encoded by a nucleic acid sequence having at least 85% sequence identity with the nucleic acid sequence of SEQ ID NO: 55. In some embodiments, the GPC3-TAC protein is encoded by a nucleic acid sequence having at least 90% sequence identity with the nucleic acid sequence of SEQ ID NO: 55. In some embodiments, the GPC3-TAC protein is encoded by a nucleic acid sequence having at least 95% sequence identity with the nucleic acid sequence of SEQ ID NO: 55. In some embodiments, the GPC3-TAC protein is encoded by a nucleic acid sequence having at least 96% sequence identity with the nucleic acid sequence of SEQ ID NO: 55. In some embodiments, the GPC3-TAC protein is encoded by a nucleic acid sequence having at least 97% sequence identity with the nucleic acid sequence of SEQ ID NO: 55. In some embodiments, the GPC3-TAC protein is encoded by a nucleic acid sequence having at least 98% sequence identity with the nucleic acid sequence of SEQ ID NO: 55. In some embodiments, the GPC3-TAC protein is encoded by a nucleic acid sequence having at least 99% sequence identity with the nucleic acid sequence of SEQ ID NO: 55. In some embodiments, the GPC3-TAC protein is encoded by a nucleic acid sequence of SEQ ID NO: 55.

In some embodiments, the GPC3-TAC protein is encoded by a nucleic acid sequence having at least 80% sequence identity with the nucleic acid sequence of SEQ ID NO: 57. In some embodiments, the GPC3-TAC protein is encoded by a nucleic acid sequence having at least 85% sequence identity with the nucleic acid sequence of SEQ ID NO: 57. In some embodiments, the GPC3-TAC protein is encoded by a nucleic acid sequence having at least 90% sequence identity with the nucleic acid sequence of SEQ ID NO: 57. In some embodiments, the GPC3-TAC protein is encoded by a nucleic acid sequence having at least 95% sequence identity with the nucleic acid sequence of SEQ ID NO: 57. In some embodiments, the GPC3-TAC protein is encoded by a nucleic acid sequence having at least 96% sequence identity with the nucleic acid sequence of SEQ ID NO: 57. In some embodiments, the GPC3-TAC protein is encoded by a nucleic acid sequence having at least 97% sequence identity with the nucleic acid sequence of SEQ ID NO: 57. In some embodiments, the GPC3-TAC protein is encoded by a nucleic acid sequence having at least 98% sequence identity with the nucleic acid sequence of SEQ ID NO: 57. In some embodiments, the GPC3-TAC protein is encoded by a nucleic acid sequence having at least 99% sequence identity with the nucleic acid sequence of SEQ ID NO: 57. In some embodiments, the GPC3-TAC protein is encoded by a nucleic acid sequence of SEQ ID NO: 57.

In some embodiments, the GPC3-TAC protein is encoded by a nucleic acid sequence having at least 80% sequence identity with the nucleic acid sequence of SEQ ID NO: 59. In some embodiments, the GPC3-TAC protein is encoded by a nucleic acid sequence having at least 85% sequence identity with the nucleic acid sequence of SEQ ID NO: 59. In some embodiments, the GPC3-TAC protein is encoded by a nucleic acid sequence having at least 90% sequence identity with the nucleic acid sequence of SEQ ID NO: 59. In some embodiments, the GPC3-TAC protein is encoded by a nucleic acid sequence having at least 95% sequence identity with the nucleic acid sequence of SEQ ID NO: 59. In some embodiments, the GPC3-TAC protein is encoded by a nucleic acid sequence having at least 96% sequence identity with the nucleic acid sequence of SEQ ID NO: 59. In some embodiments, the GPC3-TAC protein is encoded by a nucleic acid sequence having at least 97% sequence identity with the nucleic acid sequence of SEQ ID NO: 59. In some embodiments, the GPC3-TAC protein is encoded by a nucleic acid sequence having at least 98% sequence identity with the nucleic acid sequence of SEQ ID NO: 59. In some embodiments, the GPC3-TAC protein is encoded by a nucleic acid sequence having at least 99% sequence identity with the nucleic acid sequence of SEQ ID NO: 59. In some embodiments, the GPC3-TAC protein is encoded by a nucleic acid sequence of SEQ ID NO: 59.

In some embodiments, the GPC3-TAC protein is encoded by a nucleic acid sequence having at least 80% sequence identity with the nucleic acid sequence of SEQ ID NO: 61. In some embodiments, the GPC3-TAC protein is encoded by a nucleic acid sequence having at least 85% sequence identity with the nucleic acid sequence of SEQ ID NO: 61. In some embodiments, the GPC3-TAC protein is encoded by a nucleic acid sequence having at least 90% sequence identity with the nucleic acid sequence of SEQ ID NO: 61. In some embodiments, the GPC3-TAC protein is encoded by a nucleic acid sequence having at least 95% sequence identity with the nucleic acid sequence of SEQ ID NO: 61. In some embodiments, the GPC3-TAC protein is encoded by a nucleic acid sequence having at least 96% sequence identity with the nucleic acid sequence of SEQ ID NO: 61. In some embodiments, the GPC3-TAC protein is encoded by a nucleic acid sequence having at least 97% sequence identity with the nucleic acid sequence of SEQ ID NO: 61. In some embodiments, the GPC3-TAC protein is encoded by a nucleic acid sequence having at least 98% sequence identity with the nucleic acid sequence of SEQ ID NO: 61. In some embodiments, the GPC3-TAC protein is encoded by a nucleic acid sequence having at least 99% sequence identity with the nucleic acid sequence of SEQ ID NO: 61. In some embodiments, the GPC3-TAC protein is encoded by a nucleic acid sequence of SEQ ID NO: 61.

In some embodiments, the GPC3-TAC protein is encoded by a nucleic acid sequence having at least 80% sequence identity with the nucleic acid sequence of SEQ ID NO: 63. In some embodiments, the GPC3-TAC protein is encoded by a nucleic acid sequence having at least 85% sequence identity with the nucleic acid sequence of SEQ ID NO: 63. In some embodiments, the GPC3-TAC protein is encoded by a nucleic acid sequence having at least 90% sequence identity with the nucleic acid sequence of SEQ ID NO: 63. In some embodiments, the GPC3-TAC protein is encoded by a nucleic acid sequence having at least 95% sequence identity with the nucleic acid sequence of SEQ ID NO: 63. In some embodiments, the GPC3-TAC protein is encoded by a nucleic acid sequence having at least 96% sequence identity with the nucleic acid sequence of SEQ ID NO: 63. In some embodiments, the GPC3-TAC protein is encoded by a nucleic acid sequence having at least 97% sequence identity with the nucleic acid sequence of SEQ ID NO: 63. In some embodiments, the GPC3-TAC protein is encoded by a nucleic acid sequence having at least 98% sequence identity with the nucleic acid sequence of SEQ ID NO: 63. In some embodiments, the GPC3-TAC protein is encoded by a nucleic acid sequence having at least 99% sequence identity with the nucleic acid sequence of SEQ ID NO: 63. In some embodiments, the GPC3-TAC protein is encoded by a nucleic acid sequence of SEQ ID NO: 63.

Amino acid sequences of exemplary GPC3-TACs are provided in Table 6.

TABLE 6
Table of Sequences

| SEQ ID NO | Description | Nucleotide/Amino Acid |
|---|---|---|
| SEQ ID NO: 53 | GPC3-TAC GC1 | Nucleotide |
| SEQ ID NO: 54 | GPC3-TAC GC1 | Amino Acid |
| SEQ ID NO: 55 | GPC3-TAC GC2 | Nucleotide |
| SEQ ID NO: 56 | GPC3-TAC GC2 | Amino Acid |
| SEQ ID NO: 57 | GPC3-TAC GC3 | Nucleotide |
| SEQ ID NO: 58 | GPC3-TAC GC3 | Amino Acid |
| SEQ ID NO: 59 | GPC3-TAC GC4 | Nucleotide |
| SEQ ID NO: 60 | GPC3-TAC GC4 | Amino Acid |
| SEQ ID NO: 61 | GPC3-TAC GC5 | Nucleotide |
| SEQ ID NO: 62 | GPC3-TAC GC5 | Amino Acid |
| SEQ ID NO: 63 | GPC3-TAC GC6 | Nucleotide |
| SEQ ID NO: 64 | GPC3-TAC GC6 | Amino Acid |
| SEQ ID NO: 65 | GPC3-TAC GC7 | Nucleotide |
| SEQ ID NO: 66 | GPC3-TAC GC7 | Amino Acid |
| SEQ ID NO: 67 | GPC3-TAC GC8 | Nucleotide |
| SEQ ID NO: 68 | GPC3-TAC GC8 | Amino Acid |
| SEQ ID NO: 69 | GPC3-TAC GC9 | Nucleotide |
| SEQ ID NO: 70 | GPC3-TAC GC9 | Amino Acid |
| SEQ ID NO: 71 | GPC3-TAC GC10 | Nucleotide |
| SEQ ID NO: 72 | GPC3-TAC GC10 | Amino Acid |
| SEQ ID NO: 73 | GPC3-TAC GC11 | Nucleotide |
| SEQ ID NO: 74 | GPC3-TAC GC11 | Amino Acid |
| SEQ ID NO: 75 | GPC3-TAC GC12 | Nucleotide |
| SEQ ID NO: 76 | GPC3-TAC GC12 | Amino Acid |

### Vector Constructs

Disclosed herein, in certain embodiments, are vectors comprising a GPC3 TAC nucleic acid sequence as disclosed herein. In some embodiments, the vectors further comprise a promoter. In some embodiments, the promoter is functional in a mammalian cell. Promoters, regions of DNA that initiate transcription of a particular nucleic acid sequence, are well known in the art. A “promoter functional in a mammalian cell” refers to a promoter that drives expression of the associated nucleic acid sequence in a mammalian cell. A promoter that drives expression of a nucleic acid sequence is referred to as being “operably connected” to the nucleic acid sequence.

A variety of delivery vectors and expression vehicles are employed to introduce nucleic acids described herein into a cell.

Disclosed herein, in certain embodiments, are vectors comprising:
(a) a first polynucleotide encoding an antigen-binding domain that binds GPC3;
(b) a second polynucleotide encoding an antigen-binding domain that binds a protein associated with a TCR complex;
(c) a third polynucleotide encoding a T cell receptor signaling domain polypeptide; and
(d) a promoter that is functional in a mammalian cell.

In some embodiments, the first polynucleotide and third polynucleotide are fused to the second polynucleotide and the coding sequence is operably connected to the promoter. In some embodiments, the second polynucleotide and third polynucleotide are fused to the first polynucleotide and the coding sequence is operably connected to the promoter. In some embodiments, the vector is designed for expression in mammalian cells. In some embodiments, the vector is a viral vector. In some embodiments, the viral vector is a retroviral vector.

In some embodiments, vectors that are useful comprise vectors derived from retroviruses, lentiviruses, Murine Stem Cell Viruses (MSCV), pox viruses, adenoviruses, and adeno-associated viruses. Other delivery vectors that are useful comprise vectors derived from herpes simplex viruses, transposons, vaccinia viruses, human papilloma virus, Simian immunodeficiency viruses, HTLV, human foamy virus and variants thereof. Further vectors that are useful comprise vectors derived from spumaviruses, mammalian type B retroviruses, mammalian type C retroviruses, avian type C retroviruses, mammalian type D retroviruses and HTLV/BLV type retroviruses. One example of a lentiviral vector useful in the disclosed compositions and methods is the pCCL4 vector.

### Pharmaceutical Compositions

Disclosed herein, in certain embodiments, are pharmaceutical compositions comprising an engineered T cell disclosed herein (transduced with and/or expressing a GPC3 TAC polypeptide), and a pharmaceutically acceptable carrier. Pharmaceutically acceptable carriers include, but are not limited to, buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); or preservatives. In some embodiments, the engineered T cells are formulated for intravenous administration.

Pharmaceutical compositions are administered in a manner appropriate to the disease to be treated (or prevented). The quantity and frequency of administration is determined by such factors as the condition of the patient, and the type and severity of the patient's disease, although appropriate dosages are determined by clinical trials. When “an immunologically effective amount,” “an anti-tumor effective amount,” “a tumor-inhibiting effective amount,” or “therapeutic amount” is indicated, the precise amount of the compositions of the present invention to be administered is determined by a physician with consideration of individual differences in age, weight, tumor size, extent of infection or metastasis, and condition of the patient (subject).

In some embodiments, the engineered T cells and/or pharmaceutical compositions described herein are administered at a dosage of 10¹ to 10¹⁵ cells per kg body weight, 10⁴ to 10⁹ cells per kg body weight, optionally 10⁵ to 10⁸ cells per kg body weight, 10⁶ to 10⁷ cells per kg body weight or 10⁵ to 10⁶ cells per kg body weight, including all integer values within those ranges. In some embodiments, the modified T cells and/or pharmaceutical compositions described herein are administered at a dosage of greater than 10¹ cells per kg body weight. In some embodiments, the modified T cells and/or pharmaceutical compositions described herein are administered at a dosage of less than 10¹⁵ cells per kg body weight.

In some embodiments, the engineered T cells and/or pharmaceutical compositions described herein are administered at a dosage of 0.5×10⁶ cells, 2×10⁶ cells, 4×10⁶ cells, 5×10⁶ cells, 1.2×10⁷ cells, 2×10⁷ cells, 5×10⁷ cells, 2×10⁸ cells, 5×10⁸ cells, 2×10⁹ cells, 0.5-2000×10⁶ cells, 0.5-2×10⁶ cells, 0.5-2×10⁷ cells, 0.5-2×10⁸ cells, or 0.5-2×10⁹ cells, including all integer values within those ranges.

Also disclosed herein are pharmaceutical compositions comprising engineered/modified and unmodified T cells, or comprising different populations of engineered/modified T cells with or without unmodified T cells. One of ordinary skill in the art would understand that a therapeutic quantity of engineered/modified T cells need not be homogenous in nature.

In some embodiments, T cell compositions are administered multiple times at these dosages. In some embodiments, the dosage is administered a single time or multiple times, for example daily, weekly, biweekly, or monthly, hourly, or is administered upon recurrence, relapse or progression of the cancer being treated. The cells, in some embodiments, are administered by using infusion techniques that are commonly known in immunotherapy (see, e.g., Rosenberg et al., New Eng. J. of Med. 319:1676, 1988).

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium a fungus, mycoplasma, IL-2, and IL-7.

In some embodiments, the modified/engineered T cells and/or pharmaceutical compositions are administered by methods including, but not limited to, aerosol inhalation, injection, infusion, ingestion, transfusion, implantation or transplantation. The modified T cells and/or pharmaceutical compositions may be administered to a subject transarterially, subcutaneously, intradermally, intratumorally, intranodally, intrameduliary, intramuscularly, by intravenous (i.v.) injection, by intravenous (i.v.) infusion, or intraperitoneally. The modified/engineered T cells and/or pharmaceutical compositions thereof may be administered to a patient by intradermal or subcutaneous injection. The modified/engineered T cells and/or pharmaceutical compositions thereof may be administered by i.v. injection. The modified/engineered T cells and/or pharmaceutical compositions thereof may be injected directly into a tumor, lymph node, or site of infection.

A pharmaceutical composition may be prepared by known methods for the preparation of pharmaceutically acceptable compositions that are administered to subjects, such that an effective quantity of the T cells is combined in a mixture with a pharmaceutically acceptable carrier. Suitable carriers are described, for example, in Remington's Pharmaceutical Sciences (Remington's Pharmaceutical Sciences, 20^{th} ed., Mack Publishing Company, Easton, Pa., USA, 2000). On this basis, the compositions may include, albeit not exclusively, solutions of the substances in association with one or more pharmaceutically acceptable carriers or diluents, and contained in buffered solutions with a suitable pH and iso-osmotic with the physiological fluids.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, N-(1(2,3-dioleyloxy)propyl)N,N,N-trimethylammonium chloride (DOTMA), diolesylphosphotidyl-ethanolamine (DOPE), and liposomes. In some embodiments, such compositions contain a therapeutically effective amount of the compound, together with a suitable amount of carrier so as to provide the form for direct administration to the patient.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions.

A pharmaceutical composition disclosed herein may be formulated into a variety of forms and administered by a number of different means. A pharmaceutical formulation may be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. Administration includes injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration is via an injection such as an intramuscular, intravenous, subcutaneous, or intraperitoneal injection.

Liquid formulations include an oral formulation, an intravenous formulation, an intranasal formulation, an ocular formulation, an otic formulation, an aerosol, and the like. In certain embodiments, a combination of various formulations is administered. In certain embodiments a composition is formulated for an extended release profile.

### Methods of Treatment and Use

Disclosed herein, in certain embodiments, are methods of using engineered T cells disclosed herein in the treatment of a GPC3-expressing cancer in an individual in need thereof.

In some embodiments, an antigen-binding domain that binds GPC3 of a TAC polypeptide disclosed herein binds to GPC3 on a tumor cell. In some embodiments, an antigen-binding domain that binds GPC3 of a TAC polypeptide disclosed herein selectively binds to GPC3 on a tumor cell.

Disclosed herein, in certain embodiments, are methods of treating a cancer expressing GPC3 in an individual in need thereof, comprising administering to the individual an engineered T cell disclosed herein or a pharmaceutical composition comprising an engineered T cell disclosed herein.

Further disclosed herein is use of an engineered T cell disclosed herein in the preparation of a medicament to treat cancer expressing GPC3 in an individual in need thereof. Additionally disclosed herein in certain embodiments is the use of an engineered T cell disclosed herein or a pharmaceutical composition disclosed herein to treat a cancer expressing GPC3 in an individual in need thereof.

In some embodiments, the engineered T cells disclosed herein are part of a combination therapy. In some embodiments, effectiveness of a therapy disclosed herein is assessed multiple times. In some embodiments, patients are stratified based on a response to a treatment disclosed herein. In some embodiments, an effectiveness of treatment determines entrance into a trial.

In some embodiments, the engineered T cells disclosed herein are administered in combination with a lymphodepleting therapy, or are administered to a subject who has received a lymphodepleting therapy. Examples of lymphodepleting therapies include nonmyeloablative lymphodepleting chemotherapy, myeloablative lymphodepleting chemotherapy, fludarabine, cyclophosphamide, corticosteroids, alemtuzumab, total body irradiation (TBI), and any combination thereof.

Cancers that may be treated with engineered T cells disclosed herein include any form of neoplastic disease. In some embodiments, the cancer is a liver cancer (for example, HCC), gastric carcinoma, ovarian carcinoma (for example, ovarian clear cell carcinoma), melanoma, colorectal carcinoma, thyroid cancer, squamous cell carcinoma of the lung, hepatoblastoma, nephroblastoma, or yolk sac tumor.

In some embodiments, the cancer that is to be treated is a liver cancer. Liver cancer is the fifth most prevalent cancer in the world and the third most frequent cause of cancer-related death. In some embodiments, the cancer is hepatocellular carcinoma (HCC). Hepatocellular carcinoma (HCC) is the major form of liver cancer, accounting for 90% of all liver cancers, and resulting in at least 500,000 deaths per year. The overall 5-year relative survival rate for patients with liver cancer is about 15% in the U.S. Liver cancer is usually resistant to most chemotherapy drugs. The major risk factors of HCC include cirrhosis, hepatitis B- and hepatitis C-virus infection, non-alcoholic fatty liver disease/non-alcoholic steatohepatitis (NAFLD/NASH), obesity, and diabetes. GPC3 is detected in ≥80% of patients with HCC caused by hepatitis B or C.

In some embodiments, the cancer that is to be treated is a gastric carcinoma. Gastric cancer forms in the lining of the stomach/mucosa and spreads outward to the submucosa, muscle, subserosa (connective tissue), and serosa. Risk factors for gastric cancer include Helicobacter pylori (H. pylori) infection of the stomach, chronic gastritis, pernicious anemia, intestinal metaplasia, gastric polyps, and Epstein-Barr virus infection. GPC3 overexpression is associated with gastric cancers.

In some embodiments, the cancer that is to be treated is a colorectal carcinoma. Colorectal cancer, also known as bowel cancer, colon cancer, or rectal cancer, is any cancer that affects the colon and rectum. Colorectal cancer starts in the colon or the rectum. Most colorectal cancers start as a growth (aka, polyp) on the inner lining of the colon or rectum. Some types of polyps can change into cancer over time, but not all polyps become cancer. The chance of a polyp turning into cancer depends on the type of polyp it is. The American Cancer Society (ACS) expects to see around 104,270 new cases of colon cancer and 45,230 new cases of rectal cancer in the United States in 2021. GPC3 overexpression is associated with colorectal cancers.

In some embodiments, the cancer that is to be treated is a thyroid carcinoma. Thyroid cancer occurs in the cells of the thyroid. The most common form of thyroid cancer, papillary thyroid cancer arises from follicular cells. Papillary thyroid cancer can occur at any age, but most often it affects people ages 30 to 50. Follicular thyroid cancer also arises from the follicular cells of the thyroid. It usually affects people older than age 50. Anaplastic thyroid cancer is a rare type of thyroid cancer that begins in the follicular cells. It grows rapidly and is very difficult to treat. Anaplastic thyroid cancer typically occurs in adults age 60 and older. Medullary thyroid cancer begins in thyroid cells called C cells, which produce the hormone calcitonin. Elevated levels of calcitonin in the blood can indicate medullary thyroid cancer at a very early stage. Certain genetic syndromes increase the risk of medullary thyroid cancer, although this genetic link is uncommon. GPC3 overexpression is associated with thyroid cancers, especially follicular thyroid cancer and papillary thyroid cancer.

In some embodiments, the cancer that is to be treated is an ovarian cancer. In some embodiments, the cancer is ovarian clear cell carcinoma (or, clear cell ovarian carcinoma). Ovarian clear cell carcinoma is a subtype of epithelial ovarian cancer. Ovarian clear cell carcinoma constitutes about 5-10% of epithelial ovarian cancers. Incidence rates for ovarian clear cell carcinoma differ across various ethnic groups, with the highest rates among Asians at 11.1% versus whites with 4.8% and blacks at 3.1%. GPC3 overexpression is seen in ovarian clear cell carcinomas has been associated with resistance to taxane-based chemotherapy and a poor prognosis.

In some embodiments, the cancer is melanoma. Melanoma is a type of skin cancer that develops from the pigment-producing cells known as melanocytes. Melanomas typically occur in the skin but may also occur in the mouth, intestines or eye (uveal melanoma). Australia and New Zealand have the highest rates of melanoma in the world. There are also high rates in Northern Europe and North America, while it is less common in Asia, Africa and Latin America. GPC3 is overexpressed in over 40% of melanomas, especially at earlier stages of the disease.

In some embodiments, the cancer is squamous cell carcinoma of the lung. Squamous cell carcinoma of the lung is one type of non-small cell lung cancer (NSCLC). It accounts for about 30% of all lung cancers. Squamous cell lung tumors usually occur in the central part of the lung or in one of the main airways. Symptoms of the disease include cough, trouble breathing, chest pain, and blood in the sputum. Squamous cell lung cancer metastasizes to multiple sites in the body, including the brain, spine and other bones, adrenal glands, and liver. Squamous cell lung cancer is associated with smoking and exposure to secondhand smoke, mineral and metal dust, asbestos, or radon. GPC3 is overexpressed in squamous cell lung cancer, and this overexpression is significantly higher than in lung adenocarcinoma.

In some embodiments, the cancer is hepatoblastoma. Hepatoblastoma is a rare cancer found in the liver. It usually affects children less than 3 to 4 years of age. Most cases appear during the first 18 months of life. Hepatoblastoma affects white children more frequently than black children, and is more common in boys than girls up to about age 5. It occurs more frequently in children who were born very prematurely (early) with very low birth weights. GPC3 overexpression is highly associated with the diagnosis of pediatric hepatoblastoma.

In some embodiments, the cancer is nephroblastoma (Wilms tumor). Wilms' tumor is a cancer of the kidneys that typically occurs in children and rarely in adults. Approximately 650 cases are diagnosed in the U.S. annually. The overall 5-year survival is estimated to be approximately 90%. The peak age of Wilms' tumor is 3 to 4 years and most cases occur before the age of 10 years. GPC3 overexpression is associated with the presence of Wilms tumors.

In some embodiments, the cancer is yolk sac tumor. Yolk sac tumors (also known as endodermal sinus tumors) are malignant primitive germ cell tumors. Yolk sac tumors can be found in a pure form or mixed with other germ cell tumors. It is most often found in children before the ages of 1 to 2, but can occur throughout life. Yolk sac tumors most often occur in the ovaries or testicles, but may also be found in the chest, abdomen, or brain. GPC3 overexpression is associated with the presence of yolk sac tumors.

### EXAMPLES

### Example 1: Manufacturing of GPC3-TAC T Cells

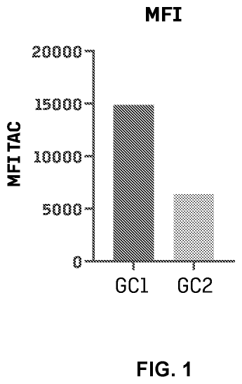

T cells were engineered with lentiviral vectors to express a variety of GPC3-TAC receptors GC1 (SEQ ID NO: 54) or GC2 (SEQ ID NO: 56). Surface expression was analyzed via flow cytometry (FIG. 1). Results show that engineered T cells expressed GPC3-TACs.

### Example 2: In Vitro Activation of GPC3-TAC T Cells Against Various Tumor Cell Types Endogenously Expressing GPC3

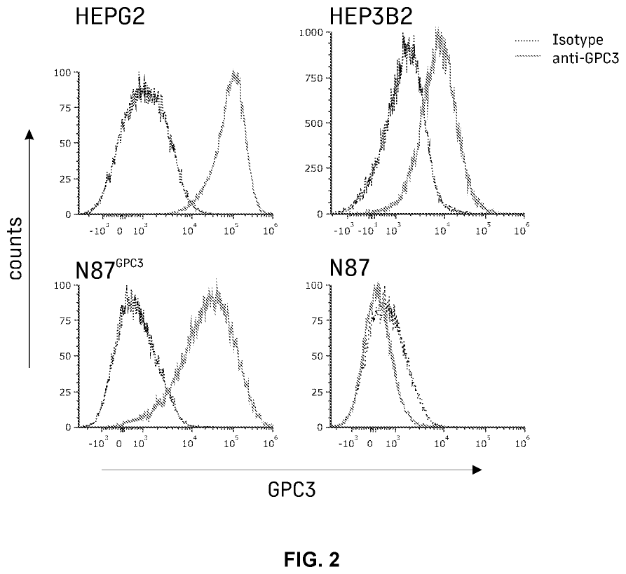

The natural surface expression of GPC3 on HEPG2 and HEP3B2 (hepatocellular carcinomas) cells was analyzed by flow cytometry. One cell line was engineered to ectopically express GPC3 (N87^{GPC3}) as a positive control. Dotted lines represent isotype controls and were used as negative controls. As shown in FIG. 2, the wild-type cell lines showed surface expression of GPC3, with HEPG2 being at levels comparable to the engineered cell line, whereas HEP3B2 had lower expression.

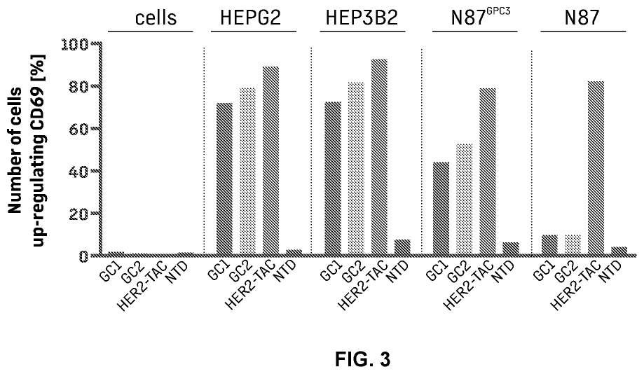

T cells were engineered to express GPC3-TAC receptors GC1 (SEQ ID NO: 54) or GC2 (SEQ ID NO: 56), with a HER2-targeting TAC used as a positive control. T cell activation was measured as a function of the upregulation of the early T cell activation marker, CD69. GPC3-TAC T cells were co-cultured at a 1:1 ratio with tumor cell lines the naturally express GPC3 and HER2 (HEPG2, HEP3B2). N87^{GPC3} was used as positive control while GPC3-negative/HER2-positive N87 cells were used as negative control. Following a 4-hour co-culture, GPC3-TAC T cells were harvested and analyzed for CD69 surface expression by flow cytometry. As shown in FIG. 3, GPC3-TAC T cells were activated when co-cultured with all target cells naturally expressing GPC3. No meaningful CD69 upregulation was observed against GPC3-negative control cells, N87. GPC3-TAC T cells were able to induce the activation of non-transduced T cells in those same T cell products.

### Example 3: In Vitro Proliferation of GPC3-TAC T Cells Following Co-Culture with Various Tumor Cell Types Endogenously Expressing GPC3

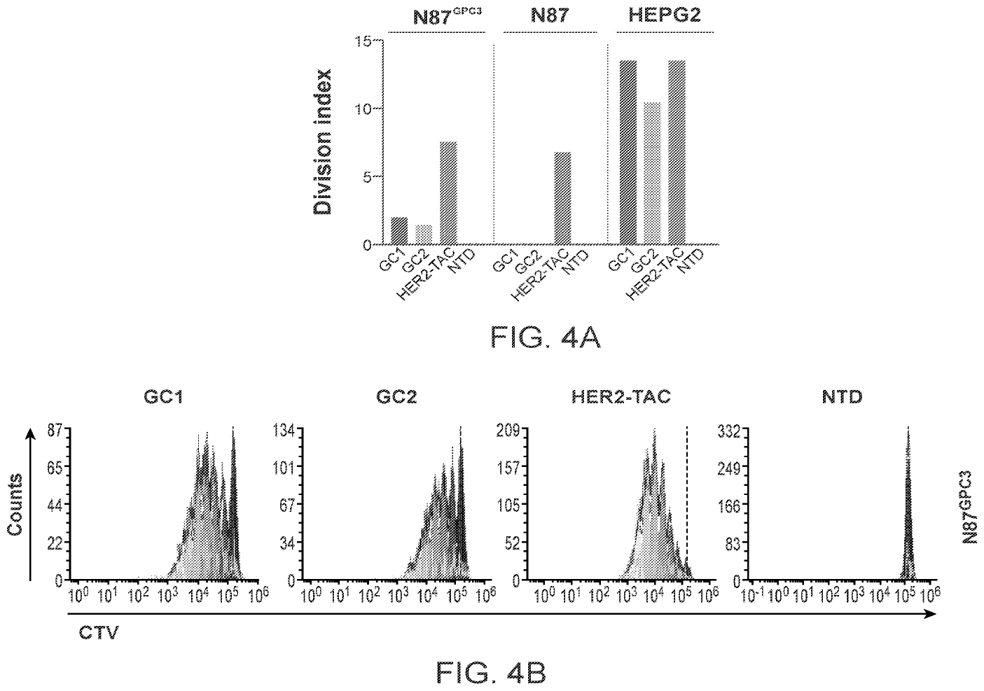

T cells were engineered to express GPC3-TAC receptors GC1 (SEQ ID NO: 54) or GC2 (SEQ ID NO: 56), with a HER2-targeting TAC used as a positive control. Proliferation of GPC3-TAC T cells co-cultured in a 1:3 E:T ratio for 4 days with N87^{GPC3}, N87 or HEPG2 target cells was evaluated. HEPG2 cells are hepatocellular carcinoma cells that naturally express GPC3 and HER2. N87^{GPC3} is a gastric carcinoma cell line that was engineered to overexpress GPC3. The GPC3-negative/HER2-positive parental N87 cell line was used as a negative control. GPC3-TAC T cells were evaluated via the CTV (cell trace violet) proliferation assay. Briefly, target cells were inactivated using mitomycin C, and T cells were loaded with CTV dye prior to co-culture. Target cells were co-cultured with T cells at an 1:3 ET ratio. After a 4-day co-culture, T cells were analyzed via flow cytometry. Results of the CTV proliferation assay were quantified (FIG. 4A), and representative examples are shown (FIG. 4B). The division index (DI), a measure of proliferation from all GPC3-TAC T cells was normalized to the division index of cells grown in the absence of target cells. The GPC3-TAC T cells both showed proliferation in response to GPC3-expressing target cells. No proliferation was observed against GPC3-negative control cells, N87. Examples of proliferating GPC3-TAC (GC1; SEQ ID NO:54 and GC2; SEQ ID NO: 56) T cells are shown (FIG. 4B). GPC3-TAC GC1 and GC2 showed various levels of proliferation relative to the positive-control of HER2-TAC T cells. No proliferation was observed in the NTD negative control cells. These results show that T cells expressing GPC3 TACs are capable of proliferation following stimulation with GPC3-expressing target cells.

### Example 4: In Vitro Cytotoxicity of GPC3-Expressing Target Cells Induced by GPC3-TACT Cells

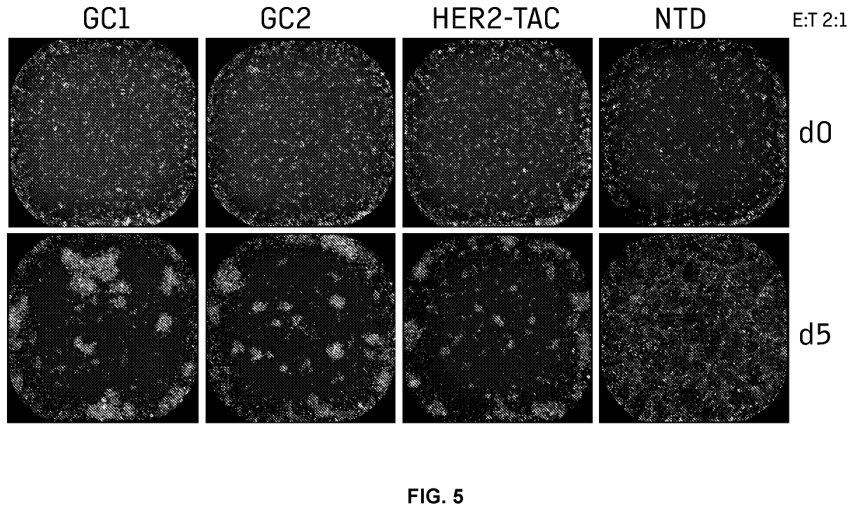

T cells were engineered to express GPC3-TAC receptors GC1 (SEQ ID NO: 54) or GC2 (SEQ ID NO: 56), with a HER2-targeting TAC used as a positive control. GPC3-TAC T cells were co-cultured at E:T ratios 2:1, 1:1, 1:10 and 1:20 with 1×10⁴ GFP-expressing N87^{GPC3-GFPeLuc} target cells/well in a cell imaging reader. GFP fluorescence images were captured every 8 hours for 5 days. FIG. 5 shows fluorescence images of target cells co-cultured with GPC3-TAC T cells, GC1 (SEQ ID NO: 54) and GC2 (SEQ ID NO: 56), as well as the HER2-TAC positive control, and the NTD negative control at an E:T of 2:1 taken at the start of the experiment (do), followed by an image taken at the endpoint, Day 5 (d5). While at d0, all target cells show an even distribution, on day 5 target cells co-cultured with control NTD cells continue to show an even distribution of target cells, expected for a healthy monolayer of adherent cells. Conversely, target cells co-cultured with either TAC T cells GC1, GC2, or the HER2-TAC positive control show punctate, aggregated cell clusters, indicating TAC T cell-mediated cytotoxicity against target cells. These results show that T cells expressing GPC3 TACs are capable of killing GPC3-expressing target cells.

### Example 5: In Vitro Screening of GPC3-TACs

T cells are engineered to express GPC3-TAC receptors (e.g., any one of SEQ ID NOs: 54, 56, 58, 60, 62, 64, 66, 68, 70, 72, 74, or 76). T cell activation is measured as a function of the upregulation of the early T cell activation marker, CD69. Engineered T cells are co-cultured at a 1:1 E:T ratio with target cells expressing GPC3 or negative control cells that do not express GPC3. Following a 4-hour co-culture, GPC3-TAC T cells are harvested and analyzed for CD69 surface expression by flow cytometry. Expansion of GPC3-TAC T cells is evaluated via the CTV proliferation assay. GPC3-positive target cells or GPC3-negative control cells are inactivated using mitomycin C, and T cells are loaded with CTV dye prior to co-culture with target or control cells at a 3:1 E:T ratio. After a 4-day co-culture, T cells are analyzed via flow cytometry. GFP/Luc-expressing GPC3-positive target cells or GPC3-negative control cells are used to assess cytotoxicity induced by TAC T cells in a cell imaging reader. Photos are captured every 8 hours for 5 days. The area of GFP-expressing cells is calculated for each time point and E:T ratio. From these values, the area under the curve (AUC) for each of the GPC3-TAC T cells is calculated and plotted, representing target cell killing at each E:T ratio. Results are analyzed to compare the relative effects of the T cells expressing GPC3 TACs on GPC3-positive target cells or GPC3-negative control cells.

### Example 6: In Vivo Activity of GPC3-TAC T Cells in Mammalian Subjects

T cells are engineered to express GPC3-TAC receptors (e.g., any one of SEQ ID NOs: 54, 56, 58, 60, 62, 64, 66, 68, 70, 72, 74, or 76). Mice are inoculated with 5×10⁵-1×10⁷ GPC3-expressing tumor cells. Four days after engraftment, mice are treated with a single intravenous dose of GPC3-TAC T cells. Non-treated (NT) mice and mice treated with non-transduced T cells (NTD) are used as negative controls. Mice are dosed with 4×10⁶ TAC T cells or an equivalent number of NTD cells that matches the total T cell dose used for TAC T cells. Total luminescence is measured weekly. The resulting total flux (photons/second) as the sum of the dorsal and ventral reads, overall survival, and relative change in body weight as a means to assess toxicity are measured. Results are analyzed to compare animals treated with GPC3-TAC T cells to NT and NTD animals.

### Example 7: Treatment of Human Subjects with GPC3-TAC T Cells

A human subject having a GPC3-expressing primary liver cancer presents. Autologous T cells are engineered to express a GPC3-TAC receptor and expression of the TAC is confirmed. The subject is administered lymphodepleting chemotherapy followed by administration of TAC-expressing T cells at an appropriate dose. The subject is monitored for toxicity and disease progression.

### Example 8: Treatment of Human Subjects Having Primary Colorectal Cancer with GPC3-TAC T Cells

A human subject having a GPC3-expressing primary colorectal cancer presents. Autologous T cells are engineered to express a GPC3-TAC receptor and expression of the TAC is confirmed. The subject is administered lymphodepleting chemotherapy followed by administration of TAC-expressing T cells at an appropriate dose. The subject is monitored for toxicity and disease progression.

### Example 9: Treatment of Human Subjects Having Hepatocellular Carcinoma with GPC3-TAC T Cells

A human subject having a GPC3-expressing hepatocellular carcinoma presents. Autologous T cells are engineered to express a GPC3-TAC receptor and expression of the TAC is confirmed. The subject is administered lymphodepleting chemotherapy followed by administration of TAC-expressing T cells at an appropriate dose. The subject is monitored for toxicity and disease progression.

### Example 10: Treatment of Human Subjects Having Squamous Cell Lung Carcinoma with GPC3-TAC T Cells

A human subject having a GPC3-expressing squamous cell lung carcinoma presents. Autologous T cells are engineered to express a GPC3-TAC receptor and expression of the TAC is confirmed. The subject is administered lymphodepleting chemotherapy followed by administration of TAC-expressing T cells at an appropriate dose. The subject is monitored for toxicity and disease progression.

Sequence Listing

| SEQ | | |
|---|---|---|
| ID | Nucleotide/ | |
| NO | Amino Acid | Sequence |
| 1 | Nucleotide | ATGGATTTCCAGGTCCAGATTTTCTCCTTCCTGCTGATTTCCGCAAGCGTCATT |
| 2 | Amino Acid | MDFQVQIFSFLLISASVI |
| 3 | Nucleotide | GAACAGAAACTGATTAGCGAAGAAGACCTG |
| 4 | Amino Acid | EQKLISEEDL |
| 5 | Nucleotide | ACTAGTGGCGGAGGAGGATCACTCGAG |
| 6 | Amino Acid | TSGGGGSLE |
| 7 | Nucleotide | AACCCCGGGGGAGGAGGAGGGAGCGGGGGAGGAGGCAGCGGCGGGGGAGGCTCTGGAG |
| | | GAGGAGGGAGCGGATCC |
| 8 | Amino Acid | NPGGGGGSGGGGSGGGGSGGGGSGS |
| 9 | Nucleotide | AGCGGACAGGTGCTGCTGGAATCCAATATCAAAGTCCTGCCCACTTGGTCTACCCCCGTG |
| | | CAGCCT |
| 10 | Amino Acid | SGQVLLESNIKVLPTWSTPVQP |
| 11 | Nucleotide | GCCGAAGCAGCAGCAAAGGAGGCCGCAGCGAAGGAAGCAGCTGCGAAGGCC |
| 12 | Amino Acid | AEAAAKEAAAKEAAAKA |
| 13 | Nucleotide | GCCGAGGCAGCTGCAAAGGAAGCTGCGGCGAAGGAGGCCGCAGCGAAAGAAGCAGCGG |
| | | CAAAAGAAGCAGCCGCCAAAGCC |
| 14 | Amino Acid | AEAAAKEAAAKEAAAKEAAAKEAAAKA |
| 15 | Nucleotide | ATCGTAGTGTTGGCATTTCAAAAAGCGTCTAGCATCGTCTATAAGAAGGAAGGTGAACAA |
| | | GTCGAGTTTTCTTTCCCCCTTGCATTTACGGTGGAAAAGCTTACGGGTAGCGGCGAGCTGT |
| | | GGTGGCAAGCTGAACGGGCTTCAAGCTCAAAATCTTGGATTACTTTTGACTTGAAGAACA |
| | | AAGAGGTGAGTGTCAAAAGAGTTACTCAGGACCCAAAGCTTCAAATGGGGAAGAAACTT |
| | | CCGCTGCACCTGACGTTGCCTCAGGCCCTGCCTCAATATGCCGGCTCAGGCAATCTGACC |
| | | CTCGCGCTGGAAGCTAAGACCGGAAAATTGCACCAGGAAGTCAATTTGGTTGTGATGCGC |
| | | GCCACTCAGCTCCAAAAAAATCTCACTTGCGAGGTATGGGGGCCTACGAGCCCAAAACTT |
| | | ATGCTGTCTTTGAAGCTTGAAAACAAGGAAGCGAAAGTTTCTAAGCGCGAGAAAGCGGT |
| | | ATGGGTTTTGAATCCTGAGGCTGGAATGTGGCAATGCCTCCTGAGCGATAGCGGGCAGGT |
| | | GCTGTTGGAGAGCAACATCAAGGTTTTGCCAGCAGCC |
| 16 | Amino Acid | IVVLAFQKASSIVYKKEGEQVEFSFPLAFTVEKLTGSGELWWQAERASSSKSWITFDLKNKEV |
| | | SVKRVTQDPKLQMGKKLPLHLTLPQALPQYAGSGNLTLALEAKTGKLHQEVNLVVMRATQL |
| | | QKNLTCEVWGPTSPKLMLSLKLENKEAKVSKREKAVWVLNPEAGMWQCLLSDSGQVLLES |
| | | NIKVLPAA |
| 17 | Nucleotide | ATGGAGACCCCCGCCCAGCTGCTGTTCCTGCTGCTGCTGTGGCTGCCCGACACCACCGGC |
| 18 | Amino Acid | METPAQLLFLLLLWLPDTTG |
| 19 | Nucleotide | ATGGCCCTGCCAGTGACCGCCCTGCTGCTGCCACTGGCCCTGCTGCTGCACGCCGCCAGA |
| | | CCC |
| 20 | Amino Acid | MALPVTALLLPLALLLHAARP |
| 21 | Nucleotide | GGATCTACCAGCGGATCCGGCAAGCCTGGCAGCGGAGAGGGATCCACAAAGGGA |
| 22 | Amino Acid | GSTSGSGKPGSGEGSTKG |
| 23 | Nucleotide | ggcggcggcggaagtggaggaggaggctcaggcggaggagggagc |
| 24 | Amino Acid | GGGGSGGGGSGGGGS |
| 25 | Nucleotide | ggaggaggagggagcgggggaggaggcagcggcgggggaggctctggaggaggagggagc |
| 26 | Amino Acid | GGGGSGGGGSGGGGSGGGGS |
| 27 | Nucleotide | GGAGGAGGAGGGAGC |
| 28 | Amino Acid | GGGGS |
| 29 | Nucleotide | ATGGCCCTGCCAGTGACCGCCCTGCTGCTGCCACTGGCCCTGCTGCTGCACGCCGCCCGG |
| | | CCT |
| 30 | Amino Acid | MALPVTALLLPLALLLHAARP |
| 31 | Nucleotide | ATGGACATCCAGATGACTCAGACCACAAGCTCCCTGTCTGCAAGTCTGGGCGACCGGGTG |
| | | ACAATCTCCTGCAGAGCCTCTCAGGATATTAGGAACTACCTGAATTGGTATCAGCAGAAA |
| | | CCTGATGGCACAGTCAAGCTGCTGATCTACTATACCAGCCGGCTGCACTCAGGCGTGCCA |
| | | AGCAAATTCTCAGGAAGCGGCTCCGGGACTGACTACTCCCTGACCATCTCTAACCTGGAG |
| | | CAGGAAGATATTGCTACCTATTTCTGCCAGCAGGGCAATACACTGCCCTGGACTTTTGCCG |
| | | GAGGCACCAAACTGGAGATCAAGGGGGGAGGCGGGAGTGGAGGCGGGGGATCAGGAGG |
| | | AGGAGGCAGCGGAGGAGGAGGGTCCGAGGTCCAGCTGCAGCAGAGCGGACCAGAACTG |
| | | GTGAAGCCCGGAGCAAGTATGAAAATCTCCTGTAAGGCCTCAGGATACAGCTTCACCGGC |
| | | TATACAATGAACTGGGTGAAACAGTCCCATGGCAAGAACCTGGAATGGATGGGGCTGATT |
| | | AATCCTTACAAAGGCGTCAGCACCTATAATCAGAAGTTTAAAGACAAGGCCACACTGACT |
| | | GTGGATAAGTCTAGTTCAACCGCTTACATGGAGCTGCTGTCCCTGACATCTGAAGACAGT |
| | | GCCGTGTACTATTGTGCTCGGTCTGGCTACTATGGGGACAGTGATTGGTACTTCGATGTCT |
| | | GGGGACAGGGCACTACCCTGACCGTGTTTTCT |
| 32 | Amino Acid | MDIQMTQTTSSLSASLGDRVTISCRASQDIRNYLNWYQQKPDGTVKLLIYYTSRLHSGVPSKF |
| | | SGSGSGTDYSLTISNLEQEDIATYFCQQGNTLPWTFAGGTKLEIKGGGGSGGGGSGGGGSGGG |
| | | GSEVQLQQSGPELVKPGASMKISCKASGYSFTGYTMNWVKQSHGKNLEWMGLINPYKGVST |
| | | YNQKFKDKATLTVDKSSSTAYMELLSLTSEDSAVYYCARSGYYGDSDWYFDVWGQGTTLTV |
| | | FS |
| 33 | Nucleotide | ATGGCCGACATCGTGCTGACACAGAGCCCCGCCATCATGTCTGCCAGCCCTGGCGAGAAA |
| | | GTGACCATGACCTGTAGCGCCAGCAGCAGCGTGTCCTACATGAACTGGTATCAGCAGAAG |
| | | TCCGGCACCAGCCCCAAGCGGTGGATCTACGACACAAGCAAGCTGGCCTCTGGCGTGCCC |
| | | GCCCACTTTAGAGGCTCTGGCAGCGGCACAAGCTACAGCCTGACCATCAGCGGCATGGAA |
| | | GCCGAGGATGCCGCCACCTACTACTGCCAGCAGTGGTCCAGCAACCCCTTCACCTTTGGC |
| | | TCCGGCACAAAGCTGGAAATCAACCGGGCCGACACCGCCCCTACAGGCGGCGGAGGATC |
| | | TGGCGGAGGCGGATCTGGGGGCGGAGGAAGTGGGGGGGGAGGATCTATGGCTCAGGTGC |
| | | AGCTGCAGCAGTCTGGCGCCGAACTGGCTAGACCTGGCGCCTCCGTGAAGATGAGCTGCA |
| | | AGGCCAGCGGCTACACCTTCACCCGGTACACCATGCACTGGGTCAAGCAGAGGCCTGGAC |
| | | AGGGCCTGGAATGGATCGGCTACATCAACCCCAGCCGGGGCTACACCAACTACAACCAG |
| | | AAGTTCAAGGACAAGGCCACCCTGACCACCGACAAGAGCAGCAGCACCGCCTACATGCA |
| | | GCTGTCCTCCCTGACCAGCGAGGACAGCGCCGTGTACTACTGCGCCCGGTACTACGACGA |
| | | CCACTACTCCCTGGACTACTGGGGCCAGGGCACCACACTGACCGTGTCTAGTA |
| 34 | Amino Acid | MADIVLTQSPAIMSASPGEKVTMTCSASSSVSYMNWYQQKSGTSPKRWIYDTSKLASGVPAH |
| | | FRGSGSGTSYSLTISGMEAEDAATYYCQQWSSNPFTFGSGTKLEINRADTAPTGGGGSGGGGS |
| | | GGGGSGGGGSMAQVQLQQSGAELARPGASVKMSCKASGYTFTRYTMHWVKQRPGQGLEWI |
| | | GYINPSRGYTNYNQKFKDKATLTTDKSSSTAYMQLSSLTSEDSAVYYCARYYDDHYSLDYW |
| | | GQGTTLTVSS |
| 35 | Nucleotide | CAGACCGTGGTGACCCAGGAGCCCAGCCTGACCGTGAGCCCCGGCGGCACCGTGACCCT |
| | | GACCTGCGGCAGCAGCACCGGCGCCGTGACCAGCGGCTACTACCCCAACTGGGTGCAGC |
| | | AGAAGCCCGGCCAGGCCCCCAGGGGCCTGATCGGCGGCACCAAGTTCCTGGCCCCCGGC |
| | | ACCCCCGCCAGGTTCAGCGGCAGCCTGCTGGGCGGCAAGGCCGCCCTGACCCTGAGCGGC |
| | | GTGCAGCCCGAGGACGAGGCCGAGTACTACTGCGCCCTGTGGTACAGCAACAGGTGGGT |
| | | GTTCGGCGGCGGCACCAAGCTGACCGTGCTGGGCGGCGGCGGCAGCGGCGGCGGCGGCA |
| | | GCGGCGGCGGCGGCAGCGAGGTGCAGCTGCTGGAGAGCGGCGGCGGCCTGGTGCAGCCC |
| | | GGCGGCAGCCTGAAGCTGAGCTGCGCCGCCAGCGGCTTCACCTTCAACATCTACGCCATG |
| | | AACTGGGTGAGGCAGGCCCCCGGCAAGGGCCTGGAGTGGGTGGCCAGGATCAGGAGCAA |
| | | GTACAACAACTACGCCACCTACTACGCCGACAGCGTGAAGAGCAGGTTCACCATCAGCA |
| | | GGGACGACAGCAAGAACACCGCCTACCTGCAGATGAACAACCTGAAGACCGAGGACACC |
| | | GCCGTGTACTACTGCGTGAGGCACGGCAACTTCGGCAACAGCTACGTGAGCTTCTTCGCC |
| | | TACTGGGGCCAGGGCACCCTGGTGACCGTGAGCAGC |
| 36 | Amino Acid | QTVVTQEPSLTVSPGGTVTLTCGSSTGAVTSGYYPNWVQQKPGQAPRGLIGGTKFLAPGTPA |
| | | RFSGSLLGGKAALTLSGVQPEDEAEYYCALWYSNRWVFGGGTKLTVLGGGGSGGGGSGGG |
| | | GSEVQLLESGGGLVQPGGSLKLSCAASGFTFNIYAMNWVRQAPGKGLEWVARIRSKYNNYA |
| | | TYYADSVKSRFTISRDDSKNTAYLQMNNLKTEDTAVYYCVRHGNFGNSYVSFFAYWGQGTL |
| | | VTVSS |
| 37 | Nucleotide | GACATCCAGCTGACCCAGAGCCCCGCCATCATGAGCGCCAGCCCCGGCGAGAAGGTGAC |
| | | CATGACCTGCAGGGCCAGCAGCAGCGTGAGCTACATGAACTGGTACCAGCAGAAGAGCG |
| | | GCACCAGCCCCAAGAGGTGGATCTACGACACCAGCAAGGTGGCCAGCGGCGTGCCCTAC |
| | | AGGTTCAGCGGCAGCGGCAGCGGCACCAGCTACAGCCTGACCATCAGCAGCATGGAGGC |
| | | CGAGGACGCCGCCACCTACTACTGCCAGCAGTGGAGCAGCAACCCCCTGACCTTCGGCGC |
| | | CGGCACCAAGCTGGAGCTGAAGGGCGGCGGCGGCAGCGGCGGCGGCGGCAGCGGCGGC |
| | | GGCGGCAGCGACATCAAGCTGCAGCAGAGCGGCGCCGAGCTGGCCAGGCCCGGCGCCAG |
| | | CGTGAAGATGAGCTGCAAGACCAGCGGCTACACCTTCACCAGGTACACCATGCACTGGGT |
| | | GAAGCAGAGGCCCGGCCAGGGCCTGGAGTGGATCGGCTACATCAACCCCAGCAGGGGCT |
| | | ACACCAACTACAACCAGAAGTTCAAGGACAAGGCCACCCTGACCACCGACAAGAGCAGC |
| | | AGCACCGCCTACATGCAGCTGAGCAGCCTGACCAGCGAGGACAGCGCCGTGTACTACTGC |
| | | GCCAGGTACTACGACGACCACTACTGCCTGGACTACTGGGGCCAGGGCACCACCCTGACC |
| | | GTGAGCAGC |
| 38 | Amino Acid | DIQLTQSPAIMSASPGEKVTMTCRASSSVSYMNWYQQKSGTSPKRWIYDTSKVASGVPYRFS |
| | | GSGSGTSYSLTISSMEAEDAATYYCQQWSSNPLTFGAGTKLELKGGGGSGGGGSGGGGSDIK |
| | | LQQSGAELARPGASVKMSCKTSGYTFTRYTMHWVKQRPGQGLEWIGYINPSRGYTNYNQKF |
| | | KDKATLTTDKSSSTAYMQLSSLTSEDSAVYYCARYYDDHYCLDYWGQGTTLTVSS |
| 39 | Nucleotide | ATGGATATCCAGATGACCCAGTCCCCGAGCTCCCTGTCCGCCTCTGTGGGCGATAGGGTC |
| | | ACCATCACCTGCCGTGCCAGTCAGGACATCCGTAATTATCTGAACTGGTATCAACAGAAA |
| | | CCAGGAAAAGCTCCGAAACTACTGATTTACTATACCTCCCGCCTGGAGTCTGGAGTCCCTT |
| | | CTCGCTTCTCTGGTTCTGGTTCTGGGACGGATTACACTCTGACCATCAGCAGTCTGCAACC |
| | | GGAAGACTTCGCAACTTATTACTGTCAGCAAGGTAATACTCTGCCGTGGACGTTCGGACA |
| | | GGGCACCAAGGTGGAGATCAAAGGCGGCGGCGGAAGTGGAGGAGGAGGCTCAGGCGGA |
| | | GGAGGGAGCGAGGTTCAGCTGGTGGAGTCTGGCGGTGGCCTGGTGCAGCCAGGGGGCTC |
| | | ACTCCGTTTGTCCTGTGCAGCTTCTGGCTACTCCTTTACCGGCTACACTATGAACTGGGTG |
| | | CGTCAGGCCCCAGGTAAGGGCCTGGAATGGGTTGCACTGATTAATCCTTATAAAGGTGTT |
| | | AGTACCTACAACCAGAAGTTCAAGGACCGTTTCACTATAAGCGTAGATAAATCCAAAAAC |
| | | ACAGCCTACCTGCAAATGAACAGCCTGCGTGCTGAGGACACTGCCGTCTATTATTGTGCT |
| | | AGAAGCGGATACTACGGCGATAGTGACTGGTATTTTGACGTGTGGGGTCAAGGAACCCTG |
| | | GTCACCGTCTCCTCG |
| 40 | Amino Acid | MDIQMTQSPSSLSASVGDRVTITCRASQDIRNYLNWYQQKPGKAPKLLIYYTSRLESGVPSRFS |
| | | GSGSGTDYTLTISSLQPEDFATYYCQQGNTLPWTFGQGTKVEIKGGGGSGGGGSGGGGSEVQ |
| | | LVESGGGLVQPGGSLRLSCAASGYSFTGYTMNWVRQAPGKGLEWVALINPYKGVSTYNQKF |
| | | KDRFTISVDKSKNTAYLQMNSLRAEDTAVYYCARSGYYGDSDWYFDVWGQGTLVTVSS |
| 41 | Nucleotide | ATGGATATCCAGATGACCCAGTCCCCGAGCTCCCTGTCCGCCTCTGTGGGCGATAGGGTC |
| | | ACCATCACCTGCCGTGCCAGTCAGGACATCCGTAATTATCTGAACTGGTATCAACAGAAA |
| | | CCAGGAAAAGCTCCGAAACTACTGATTTACTATACCTCCCGCCTGGAGTCTGGAGTCCCTT |
| | | CTCGCTTCTCTGGTTCTGGTTCTGGGACGGATTACACTCTGACCATCAGCAGTCTGCAACC |
| | | GGAAGACTTCGCAACTTATTACTGTCAGCAAGGTAATACTCTGCCGTGGACGTTCGGACA |
| | | GGGCACCAAGGTGGAGATCAAAGGCGGCGGCGGAAGTGGAGGAGGAGGCTCAGGCGGA |
| | | GGAGGGAGCGAGGTTCAGCTGGTGGAGTCTGGCGGTGGCCTGGTGCAGCCAGGGGGCTC |
| | | ACTCCGTTTGTCCTGTGCAGCTTCTGGCTACTCCTTTACCGGCTACACTATGAACTGGGTG |
| | | CGTCAGGCCCCAGGTAAGGGCCTGGAATGGGTTGCACTGATTAATCCTACCAAAGGTGTT |
| | | AGTACCTACAACCAGAAGTTCAAGGACCGTTTCACTATAAGCGTAGATAAATCCAAAAAC |
| | | ACAGCCTACCTGCAAATGAACAGCCTGCGTGCTGAGGACACTGCCGTCTATTATTGTGCT |
| | | AGAAGCGGATACTACGGCGATAGTGACTGGTATTTTGACGTGTGGGGTCAAGGAACCCTG |
| | | GTCACCGTCTCCTCG |
| 42 | Amino Acid | MDIQMTQSPSSLSASVGDRVTITCRASQDIRNYLNWYQQKPGKAPKLLIYYTSRLESGVPSRFS |
| | | GSGSGTDYTLTISSLQPEDFATYYCQQGNTLPWTFGQGTKVEIKGGGGSGGGGSGGGGSEVQ |
| | | LVESGGGLVQPGGSLRLSCAASGYSFTGYTMNWVRQAPGKGLEWVALINPTKGVSTYNQKF |
| | | KDRFTISVDKSKNTAYLQMNSLRAEDTAVYYCARSGYYGDSDWYFDVWGQGTLVTVSS |
| 43 | Nucleotide | ATGGACATCCAGATGACTCAGACCACAAGCTCCCTGTCTGCAAGTCTGGGCGACCGGGTG |
| | | ACAATCTCCTGCAGAGCCTCTCAGGATATTAGGAACTACCTGAATTGGTATCAGCAGAAA |
| | | CCTGATGGCACAGTCAAGCTGCTGATCTACTATACCAGCCGGCTGCACTCAGGCGTGCCA |
| | | AGCAAATTCTCAGGAAGCGGCTCCGGGACTGACTACTCCCTGACCATCTCTAACCTGGAG |
| | | CAGGAAGATATTGCTACCTATTTCTGCCAGCAGGGCAATACACTGCCCTGGACTTTTGCCG |
| | | GAGGCACCAAACTGGAGATCAAGGGGGGAGGCGGGAGTGGAGGCGGGGGATCAGGAGG |
| | | AGGAGGCAGCGGAGGAGGAGGGTCCGAGGTCCAGCTGCAGCAGAGCGGACCAGAACTG |
| | | GTGAAGCCCGGAGCAAGTATGAAAATCTCCTGTAAGGCCTCAGGATACAGCTTCACCGGC |
| | | TATACAATGAACTGGGTGAAACAGTCCCATGGCAAGAACCTGGAATGGATGGGGCTGATT |
| | | AATCCTACCAAAGGCGTCAGCACCTATAATCAGAAGTTTAAAGACAAGGCCACACTGACT |
| | | GTGGATAAGTCTAGTTCAACCGCTTACATGGAGCTGCTGTCCCTGACATCTGAAGACAGT |
| | | GCCGTGTACTATTGTGCTCGGTCTGGCTACTATGGGGACAGTGATTGGTACTTCGATGTCT |
| | | GGGGACAGGGCACTACCCTGACCGTGTTTTCT |
| 44 | Amino Acid | MDIQMTQTTSSLSASLGDRVTISCRASQDIRNYLNWYQQKPDGTVKLLIYYTSRLHSGVPSKF |
| | | SGSGSGTDYSLTISNLEQEDIATYFCQQGNTLPWTFAGGTKLEIKGGGGSGGGGSGGGGSGGG |
| | | GSEVQLQQSGPELVKPGASMKISCKASGYSFTGYTMNWVKQSHGKNLEWMGLINPTKGVST |
| | | YNQKFKDKATLTVDKSSSTAYMELLSLTSEDSAVYYCARSGYYGDSDWYFDVWGQGTTLTV |
| | | FS |
| 45 | Nucleotide | AGCGGACAGGTGCTGCTGGAATCCAATATCAAAGTCCTGCCCACTTGGTCTACCCCCGTG |
| | | CAGCCTATGGCTCTGATTGTGCTGGGAGGAGTCGCAGGACTGCTGCTGTTTATCGGGCTG |
| | | GGAATTTTCTTTTGCGTGCGCTGCCGGCACCGGAGAAGGCAGGCCGAGCGCATGAGCCAG |
| | | ATCAAGCGACTGCTGAGCGAGAAGAAAACCTGTCAGTGTCCCCATAGATTCCAGAAGACC |
| | | TGTTCACCCATT |
| 46 | Amino Acid | SGQVLLESNIKVLPTWSTPVQPMALIVLGGVAGLLLFIGLGIFFCVRCRHRRRQAERMSQIKRL |
| | | LSEKKTCQCPHRFQKTCSPI |
| 47 | Nucleotide | CTCGAGCTGAGGCCCGAGGCTTCTAGACCTGCTGCCGGCGGAGCCGTGCACACCAGAGGC |
| | | CTGGACTTCGCCAGCGACATCTACATCTGGGCCCCTCTGGCCGGCACCTGTGGCGTGCTG |
| | | CTGCTGAGCCTGGTCATCACCCTGTACTGCAACCACCGGAACCGGCGGAGAGTGTGCAAG |
| | | TGCCCCAGACCCGTGGTCAAGAGCGGCGACAAGCCCAGCCTGAGCGCCAGATACGTG |
| 48 | Amino Acid | LELRPEASRPAAGGAVHTRGLDFASDIYIWAPLAGTCGVLLLSLVITLYCNHRNRRRVCKCPR |
| | | PVVKSGDKPSLSARYV |
| 49 | Nucleotide | CTCGAGCTGAGGCCCGAGGCTTCTAGACCTGCTGCCGGCGGAGCCGTGCACACCAGAGGC |
| | | CTGGACTTCGCCAGCGACATCTACATCTGGGCCCCTCTGGCCGGCACCTGTGGCGTGCTG |
| | | CTGCTGAGCCTGGTCATCACCCTGTACCTGTGCTGCAGACGGCGGAGAGTGTGCAAGTGC |
| | | CCCAGACCCGTGGTCAAGAGCGGCGACAAGCCCAGCCTGAGCGCCAGATACGTG |
| 50 | Amino Acid | LELRPEASRPAAGGAVHTRGLDFASDIYIWAPLAGTCGVLLLSLVITLYLCCRRRRVCKCPRP |
| | | VVKSGDKPSLSARYV |
| 51 | Nucleotide | CTCGAGAAGAAGTCCACCCTGAAGAAACGGGTGTCCCGGCTGCCCAGACCCGAGACACA |
| | | GAAGGGCCCCCTGAGCAGCCCTATCACCCTGGGACTGCTGGTGGCCGGCGTGCTGGTGCT |
| | | GCTGGTGTCTCTGGGAGTGGCCATCCACCTGTGCTGCCGGCGGAGAAGGGCCTGCAAGTG |
| | | CCCCAGACTGCGGTTCATGAAGCAGTTCTACAAG |
| 52 | Amino Acid | LEKKSTLKKRVSRLPRPETQKGPLSSPITLGLLVAGVLVLLVSLGVAIHLCCRRRRACKCPRLR |
| | | FMKQFYK |
| 53 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGGCAGCGGCGGCGGCCTGGTGCAGCCCGAGGGCAGCCTGAAG |
| | | CTGAGCTGCGCCGCCAGCGGCTTCACCTTCAAGACCAACGCCATGAACTGGGTGAGGCAG |
| | | GCCCCCGGCAAGGGCCTGGAGTGGGTGGCCAGGATCAGGAACAAGACCAACAACTACGC |
| | | CACCTACTACGCCGACAGCGTGAAGGACAGGTTCACCATCAGCAGGGACGACAGCCAGA |
| | | GCATGCTGTACCTGCAGATGAACAACCTGAAGACCGAGGACACCGCCATGTACTTCTGCG |
| | | TGGCCGGCAACAGCTTCGCCTACTGGGGCCAGGGCACCCTGGTGACCGTGAGCGCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAAGGTGACCGTGAACTGC |
| | | AAGAGCAGCCAGAGCCTGCTGTACAGCAACAACCAGAAGAACTACCTGGCCTGGTACCA |
| | | GCAGAAGCCCGGCCAGAGCCCCAAGCTGCTGATCTACTGGGCCAGCACCAGGGAGTACG |
| | | GCGTGCCCGACAGGTTCACCGGCAGCGGCAGCGGCACCGACTTCACCCTGACCATCAGCA |
| | | GCGTGAAGGCCGAGGACCTGGCCGTGTACTACTGCCAGCAGTACTACAACTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTTATAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 54 | Amino Acid | MALPVTALLLPLALLLHAARPEVQLVGSGGGLVQPEGSLKLSCAASGFTFKTNAMNWVRQA |
| | | PGKGLEWVARIRNKTNNYATYYADSVKDRFTISRDDSQSMLYLQMNNLKTEDTAMYFCVAG |
| | | NSFAYWGQGTLVTVSAGSTSGSGKPGSGEGSTKGDIVMSQSPSSLAVSVGEKVTVNCKSSQS |
| | | LLYSNNQKNYLAWYQQKPGQSPKLLIYWASTREYGVPDRFTGSGSGTDFTLTISSVKAEDLA |
| | | VYYCQQYYNYPLTFGAGTKLELKEQKLISEEDLNPGGGGGSGGGGSGGGGSGGGGSGSMDI |
| | | QMTQSPSSLSASVGDRVTITCRASQDIRNYLNWYQQKPGKAPKLLIYYTSRLESGVPSRFSGS |
| | | GSGTDYTLTISSLQPEDFATYYCQQGNTLPWTFGQGTKVEIKGGGGSGGGGSGGGGSEVQLV |
| | | ESGGGLVQPGGSLRLSCAASGYSFTGYTMNWVRQAPGKGLEWVALINPYKGVSTYNQKFKD |
| | | RFTISVDKSKNTAYLQMNSLRAEDTAVYYCARSGYYGDSDWYFDVWGQGTLVTVSSTSGGG |
| | | GSLESGQVLLESNIKVLPTWSTPVQPMALIVLGGVAGLLLFIGLGIFFCVRCRHRRRQAERMSQ |
| | | IKRLLSEKKTCQCPHRFQKTCSPI |
| 55 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAAGGTG |
| | | ACCGTGAACTGCAAGAGCAGCCAGAGCCTGCTGTACAGCAACAACCAGAAGAACTACCT |
| | | GGCCTGGTACCAGCAGAAGCCCGGCCAGAGCCCCAAGCTGCTGATCTACTGGGCCAGCA |
| | | CCAGGGAGTACGGCGTGCCCGACAGGTTCACCGGCAGCGGCAGCGGCACCGACTTCACC |
| | | CTGACCATCAGCAGCGTGAAGGCCGAGGACCTGGCCGTGTACTACTGCCAGCAGTACTAC |
| | | AACTACCCCCTGACCTTCGGCGCCGGCACCAAGCTGGAGCTGAAGGGGTCAACCTCTGGT |
| | | AGCGGTAAGCCTGGCTCCGGCGAAGGCTCCACAAAGGGTGAGGTGCAGCTGGTGGGCAG |
| | | CGGCGGCGGCCTGGTGCAGCCCGAGGGCAGCCTGAAGCTGAGCTGCGCCGCCAGCGGCT |
| | | TCACCTTCAAGACCAACGCCATGAACTGGGTGAGGCAGGCCCCCGGCAAGGGCCTGGAG |
| | | TGGGTGGCCAGGATCAGGAACAAGACCAACAACTACGCCACCTACTACGCCGACAGCGT |
| | | GAAGGACAGGTTCACCATCAGCAGGGACGACAGCCAGAGCATGCTGTACCTGCAGATGA |
| | | ACAACCTGAAGACCGAGGACACCGCCATGTACTTCTGCGTGGCCGGCAACAGCTTCGCCT |
| | | ACTGGGGCCAGGGCACCCTGGTGACCGTGAGCGCCGAGCAGAAACTGATCAGCGAGGAA |
| | | GATCTGAATCCTGGCGGAGGCGGAGGAAGTGGTGGCGGAGGTTCTGGTGGCGGTGGATC |
| | | AGGCGGTGGCGGATCTGGATCTATGGATATCCAGATGACCCAGTCCCCGAGCTCCCTGTC |
| | | CGCCTCTGTGGGCGATAGGGTCACCATCACCTGCCGTGCCAGTCAGGACATCCGTAATTA |
| | | TCTGAACTGGTATCAACAGAAACCAGGAAAAGCTCCGAAACTACTGATTTACTATACCTC |
| | | CCGCCTGGAGTCTGGAGTCCCTTCTCGCTTCTCTGGTTCTGGTTCTGGGACGGATTACACT |
| | | CTGACCATCAGCAGTCTGCAACCGGAAGACTTCGCAACTTATTACTGTCAGCAAGGTAAT |
| | | ACTCTGCCGTGGACGTTCGGACAGGGCACCAAGGTGGAGATCAAAGGCGGCGGCGGAAG |
| | | TGGAGGAGGAGGCTCAGGCGGAGGAGGGAGCGAGGTTCAGCTGGTGGAGTCTGGCGGTG |
| | | GCCTGGTGCAGCCAGGGGGCTCACTCCGTTTGTCCTGTGCAGCTTCTGGCTACTCCTTTAC |
| | | CGGCTACACTATGAACTGGGTGCGTCAGGCCCCAGGTAAGGGCCTGGAATGGGTTGCACT |
| | | GATTAATCCTTATAAAGGTGTTAGTACCTACAACCAGAAGTTCAAGGACCGTTTCACTAT |
| | | AAGCGTAGATAAATCCAAAAACACAGCCTACCTGCAAATGAACAGCCTGCGTGCTGAGG |
| | | ACACTGCCGTCTATTATTGTGCTAGAAGCGGATACTACGGCGATAGTGACTGGTATTTTGA |
| | | CGTGTGGGGTCAAGGAACCCTGGTCACCGTCTCCTCGACATCTGGCGGCGGAGGATCTCT |
| | | GGAATCTGGACAGGTGCTGCTGGAAAGCAACATCAAGGTGCTGCCCACCTGGTCTACCCC |
| | | AGTTCAGCCTATGGCTCTGATTGTGCTTGGCGGAGTTGCCGGCCTGCTGCTCTTTATCGGC |
| | | CTGGGCATCTTCTTTTGCGTGCGGTGCAGACATCGGCGGAGACAGGCTGAGAGAATGAGC |
| | | CAGATCAAGCGGCTGCTGAGCGAGAAGAAAACCTGTCAGTGCCCTCACCGGTTCCAGAA |
| | | AACATGCAGCCCCATC |
| 56 | Amino Acid | MALPVTALLLPLALLLHAARPDIVMSQSPSSLAVSVGEKVTVNCKSSQSLLYSNNQKNYLAW |
| | | YQQKPGQSPKLLIYWASTREYGVPDRFTGSGSGTDFTLTISSVKAEDLAVYYCQQYYNYPLTF |
| | | GAGTKLELKGSTSGSGKPGSGEGSTKGEVQLVGSGGGLVQPEGSLKLSCAASGFTFKTNAMN |
| | | WVRQAPGKGLEWVARIRNKTNNYATYYADSVKDRFTISRDDSQSMLYLQMNNLKTEDTAM |
| | | YFCVAGNSFAYWGQGTLVTVSAEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPYKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 57 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGAGACCGGCGGCGGCCTGGTGCAGCCCAAGGGCAGCCTGAAG |
| | | CTGAGCTGCGCCGCCAGCGGCTTCACCTTCAACACCAACGCCATGAACTGGGTGAGGCAG |
| | | GCCCCCGGCAAGGGCCTGCAGTGGGTGGCCAGGGTGAGGAACAAGACCAACAACTACGC |
| | | CACCTACTACGCCGACAGCGTGAAGGACAGGTTCACCATCAGCAGGGACGACAGCCAGA |
| | | GCATGCTGTACCTGCAGATGAACAACCTGAAGACCGAGGACACCGCCATCTACTACTGCG |
| | | TGGGCGGCAACAGCTTCGCCTACTGGGGCCAGGGCACCCTGGTGACCGTGAGCGCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAAGGTGACCATGAGCTGC |
| | | AAGAGCAGCCAGAGCCTGCTGTACAGCAACAACCAGAAGAACTACCTGGCCTGGTACCA |
| | | CCAGAAGCCCGGCCAGAGCCCCAAGCTGCTGATCTACTGGGCCAGCACCAGGGAGAGCG |
| | | GCGTGCCCGACAGGTTCACCGGCAGCGGCAGCGGCACCGACTTCACCCTGACCATCAGCA |
| | | GCGTGAAGGCCGAGGACCTGGCCGTGTACTACTGCCAGCAGTACTACAGCTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTTATAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 58 | Amino Acid | MALPVTALLLPLALLLHAARPEVQLVETGGGLVQPKGSLKLSCAASGFTFNTNAMNWVRQA |
| | | PGKGLQWVARVRNKTNNYATYYADSVKDRFTISRDDSQSMLYLQMNNLKTEDTAIYYCVG |
| | | GNSFAYWGQGTLVTVSAGSTSGSGKPGSGEGSTKGDIVMSQSPSSLAVSVGEKVTMSCKSSQ |
| | | SLLYSNNQKNYLAWYHQKPGQSPKLLIYWASTRESGVPDRFTGSGSGTDFTLTISSVKAEDLA |
| | | VYYCQQYYSYPLTFGAGTKLELKEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPYKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 59 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAAGGTG |
| | | ACCATGAGCTGCAAGAGCAGCCAGAGCCTGCTGTACAGCAACAACCAGAAGAACTACCT |
| | | GGCCTGGTACCACCAGAAGCCCGGCCAGAGCCCCAAGCTGCTGATCTACTGGGCCAGCAC |
| | | CAGGGAGAGCGGCGTGCCCGACAGGTTCACCGGCAGCGGCAGCGGCACCGACTTCACCC |
| | | TGACCATCAGCAGCGTGAAGGCCGAGGACCTGGCCGTGTACTACTGCCAGCAGTACTACA |
| | | GCTACCCCCTGACCTTCGGCGCCGGCACCAAGCTGGAGCTGAAGGGGTCAACCTCTGGTA |
| | | GCGGTAAGCCTGGCTCCGGCGAAGGCTCCACAAAGGGTGAGGTGCAGCTGGTGGAGACC |
| | | GGCGGCGGCCTGGTGCAGCCCAAGGGCAGCCTGAAGCTGAGCTGCGCCGCCAGCGGCTT |
| | | CACCTTCAACACCAACGCCATGAACTGGGTGAGGCAGGCCCCCGGCAAGGGCCTGCAGT |
| | | GGGTGGCCAGGGTGAGGAACAAGACCAACAACTACGCCACCTACTACGCCGACAGCGTG |
| | | AAGGACAGGTTCACCATCAGCAGGGACGACAGCCAGAGCATGCTGTACCTGCAGATGAA |
| | | CAACCTGAAGACCGAGGACACCGCCATCTACTACTGCGTGGGCGGCAACAGCTTCGCCTA |
| | | CTGGGGCCAGGGCACCCTGGTGACCGTGAGCGCCGAGCAGAAACTGATCAGCGAGGAAG |
| | | ATCTGAATCCTGGCGGAGGCGGAGGAAGTGGTGGCGGAGGTTCTGGTGGCGGTGGATCA |
| | | GGCGGTGGCGGATCTGGATCTATGGATATCCAGATGACCCAGTCCCCGAGCTCCCTGTCC |
| | | GCCTCTGTGGGCGATAGGGTCACCATCACCTGCCGTGCCAGTCAGGACATCCGTAATTAT |
| | | CTGAACTGGTATCAACAGAAACCAGGAAAAGCTCCGAAACTACTGATTTACTATACCTCC |
| | | CGCCTGGAGTCTGGAGTCCCTTCTCGCTTCTCTGGTTCTGGTTCTGGGACGGATTACACTC |
| | | TGACCATCAGCAGTCTGCAACCGGAAGACTTCGCAACTTATTACTGTCAGCAAGGTAATA |
| | | CTCTGCCGTGGACGTTCGGACAGGGCACCAAGGTGGAGATCAAAGGCGGCGGCGGAAGT |
| | | GGAGGAGGAGGCTCAGGCGGAGGAGGGAGCGAGGTTCAGCTGGTGGAGTCTGGCGGTGG |
| | | CCTGGTGCAGCCAGGGGGCTCACTCCGTTTGTCCTGTGCAGCTTCTGGCTACTCCTTTACC |
| | | GGCTACACTATGAACTGGGTGCGTCAGGCCCCAGGTAAGGGCCTGGAATGGGTTGCACTG |
| | | ATTAATCCTTATAAAGGTGTTAGTACCTACAACCAGAAGTTCAAGGACCGTTTCACTATA |
| | | AGCGTAGATAAATCCAAAAACACAGCCTACCTGCAAATGAACAGCCTGCGTGCTGAGGA |
| | | CACTGCCGTCTATTATTGTGCTAGAAGCGGATACTACGGCGATAGTGACTGGTATTTTGAC |
| | | GTGTGGGGTCAAGGAACCCTGGTCACCGTCTCCTCGACATCTGGCGGCGGAGGATCTCTG |
| | | GAATCTGGACAGGTGCTGCTGGAAAGCAACATCAAGGTGCTGCCCACCTGGTCTACCCCA |
| | | GTTCAGCCTATGGCTCTGATTGTGCTTGGCGGAGTTGCCGGCCTGCTGCTCTTTATCGGCC |
| | | TGGGCATCTTCTTTTGCGTGCGGTGCAGACATCGGCGGAGACAGGCTGAGAGAATGAGCC |
| | | AGATCAAGCGGCTGCTGAGCGAGAAGAAAACCTGTCAGTGCCCTCACCGGTTCCAGAAA |
| | | ACATGCAGCCCCATC |
| 60 | Amino Acid | MALPVTALLLPLALLLHAARPDIVMSQSPSSLAVSVGEKVTMSCKSSQSLLYSNNQKNYLAW |
| | | YHQKPGQSPKLLIYWASTRESGVPDRFTGSGSGTDFTLTISSVKAEDLAVYYCQQYYSYPLTF |
| | | GAGTKLELKGSTSGSGKPGSGEGSTKGEVQLVETGGGLVQPKGSLKLSCAASGFTFNTNAMN |
| | | WVRQAPGKGLQWVARVRNKTNNYATYYADSVKDRFTISRDDSQSMLYLQMNNLKTEDTAI |
| | | YYCVGGNSFAYWGQGTLVTVSAEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPYKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 61 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGAGACCGGCGGCGGCCTGGTGCAGCCCGAGGGCAGCCTGAAG |
| | | CTGAGCTGCGCCACCAGCGGCTTCAACTTCAACACCAACGCCATGAACTGGGTGAGGCAG |
| | | GCCCCCGGCAAGGGCCTGGAGTGGGTGGCCAGGGTGAGGAACAAGACCAACAACTACGC |
| | | CACCTACTACGCCGACAGCGTGAAGGACAGGTTCACCATCAGCAGGGACGACAGCCAGA |
| | | GGATGGTGTTCCTGCAGATGAACAACCTGAAGACCGAGGACACCGCCATCTACTACTGCG |
| | | TGGCCGGCAACAGCTTCGCCTACTGGGGCCAGGGCACCCTGGTGACCGTGAGCCCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAGGGTGACCATGAACTGC |
| | | AAGAGCAGCCAGAGCCTGCTGTACAGCAGCAACCAGAAGAACTACCTGGCCTGGTACCA |
| | | GCAGAAGCCCGGCCAGAGCCCCAAGCTGCTGATCTACTGGGCCAGCACCAGGGAGAGCG |
| | | GCGTGCCCGACAGGTTCACCGGCAGCGTGAGCGGCACCGACTTCACCCTGACCATCAGCA |
| | | GCGTGAAGGCCGAGGACCTGGCCGTGTACTACTGCCAGCAGTACTACAACTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTTATAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 62 | Amino Acid | MALPVTALLLPLALLLHAARPEVQLVETGGGLVQPEGSLKLSCATSGFNFNTNAMNWVRQA |
| | | PGKGLEWVARVRNKTNNYATYYADSVKDRFTISRDDSQRMVFLQMNNLKTEDTAIYYCVA |
| | | GNSFAYWGQGTLVTVSPGSTSGSGKPGSGEGSTKGDIVMSQSPSSLAVSVGERVTMNCKSSQ |
| | | SLLYSSNQKNYLAWYQQKPGQSPKLLIYWASTRESGVPDRFTGSVSGTDFTLTISSVKAEDLA |
| | | VYYCQQYYNYPLTFGAGTKLELKEQKLISEEDLNPGGGGGSGGGGSGGGGSGGGGSGSMDI |
| | | QMTQSPSSLSASVGDRVTITCRASQDIRNYLNWYQQKPGKAPKLLIYYTSRLESGVPSRFSGS |
| | | GSGTDYTLTISSLQPEDFATYYCQQGNTLPWTFGQGTKVEIKGGGGSGGGGSGGGGSEVQLV |
| | | ESGGGLVQPGGSLRLSCAASGYSFTGYTMNWVRQAPGKGLEWVALINPYKGVSTYNQKFKD |
| | | RFTISVDKSKNTAYLQMNSLRAEDTAVYYCARSGYYGDSDWYFDVWGQGTLVTVSSTSGGG |
| | | GSLESGQVLLESNIKVLPTWSTPVQPMALIVLGGVAGLLLFIGLGIFFCVRCRHRRRQAERMSQ |
| | | IKRLLSEKKTCQCPHRFQKTCSPI |
| 63 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAGGGTG |
| | | ACCATGAACTGCAAGAGCAGCCAGAGCCTGCTGTACAGCAGCAACCAGAAGAACTACCT |
| | | GGCCTGGTACCAGCAGAAGCCCGGCCAGAGCCCCAAGCTGCTGATCTACTGGGCCAGCA |
| | | CCAGGGAGAGCGGCGTGCCCGACAGGTTCACCGGCAGCGTGAGCGGCACCGACTTCACC |
| | | CTGACCATCAGCAGCGTGAAGGCCGAGGACCTGGCCGTGTACTACTGCCAGCAGTACTAC |
| | | AACTACCCCCTGACCTTCGGCGCCGGCACCAAGCTGGAGCTGAAGGGGTCAACCTCTGGT |
| | | AGCGGTAAGCCTGGCTCCGGCGAAGGCTCCACAAAGGGTGAGGTGCAGCTGGTGGAGAC |
| | | CGGCGGCGGCCTGGTGCAGCCCGAGGGCAGCCTGAAGCTGAGCTGCGCCACCAGCGGCT |
| | | TCAACTTCAACACCAACGCCATGAACTGGGTGAGGCAGGCCCCCGGCAAGGGCCTGGAG |
| | | TGGGTGGCCAGGGTGAGGAACAAGACCAACAACTACGCCACCTACTACGCCGACAGCGT |
| | | GAAGGACAGGTTCACCATCAGCAGGGACGACAGCCAGAGGATGGTGTTCCTGCAGATGA |
| | | ACAACCTGAAGACCGAGGACACCGCCATCTACTACTGCGTGGCCGGCAACAGCTTCGCCT |
| | | ACTGGGGCCAGGGCACCCTGGTGACCGTGAGCCCCGAGCAGAAACTGATCAGCGAGGAA |
| | | GATCTGAATCCTGGCGGAGGCGGAGGAAGTGGTGGCGGAGGTTCTGGTGGCGGTGGATC |
| | | AGGCGGTGGCGGATCTGGATCTATGGATATCCAGATGACCCAGTCCCCGAGCTCCCTGTC |
| | | CGCCTCTGTGGGCGATAGGGTCACCATCACCTGCCGTGCCAGTCAGGACATCCGTAATTA |
| | | TCTGAACTGGTATCAACAGAAACCAGGAAAAGCTCCGAAACTACTGATTTACTATACCTC |
| | | CCGCCTGGAGTCTGGAGTCCCTTCTCGCTTCTCTGGTTCTGGTTCTGGGACGGATTACACT |
| | | CTGACCATCAGCAGTCTGCAACCGGAAGACTTCGCAACTTATTACTGTCAGCAAGGTAAT |
| | | ACTCTGCCGTGGACGTTCGGACAGGGCACCAAGGTGGAGATCAAAGGCGGCGGCGGAAG |
| | | TGGAGGAGGAGGCTCAGGCGGAGGAGGGAGCGAGGTTCAGCTGGTGGAGTCTGGCGGTG |
| | | GCCTGGTGCAGCCAGGGGGCTCACTCCGTTTGTCCTGTGCAGCTTCTGGCTACTCCTTTAC |
| | | CGGCTACACTATGAACTGGGTGCGTCAGGCCCCAGGTAAGGGCCTGGAATGGGTTGCACT |
| | | GATTAATCCTTATAAAGGTGTTAGTACCTACAACCAGAAGTTCAAGGACCGTTTCACTAT |
| | | AAGCGTAGATAAATCCAAAAACACAGCCTACCTGCAAATGAACAGCCTGCGTGCTGAGG |
| | | ACACTGCCGTCTATTATTGTGCTAGAAGCGGATACTACGGCGATAGTGACTGGTATTTTGA |
| | | CGTGTGGGGTCAAGGAACCCTGGTCACCGTCTCCTCGACATCTGGCGGCGGAGGATCTCT |
| | | GGAATCTGGACAGGTGCTGCTGGAAAGCAACATCAAGGTGCTGCCCACCTGGTCTACCCC |
| | | AGTTCAGCCTATGGCTCTGATTGTGCTTGGCGGAGTTGCCGGCCTGCTGCTCTTTATCGGC |
| | | CTGGGCATCTTCTTTTGCGTGCGGTGCAGACATCGGCGGAGACAGGCTGAGAGAATGAGC |
| | | CAGATCAAGCGGCTGCTGAGCGAGAAGAAAACCTGTCAGTGCCCTCACCGGTTCCAGAA |
| | | AACATGCAGCCCCATC |
| 64 | Amino Acid | MALPVTALLLPLALLLHAARPDIVMSQSPSSLAVSVGERVTMNCKSSQSLLYSSNQKNYLAW |
| | | YQQKPGQSPKLLIYWASTRESGVPDRFTGSVSGTDFTLTISSVKAEDLAVYYCQQYYNYPLTF |
| | | GAGTKLELKGSTSGSGKPGSGEGSTKGEVQLVETGGGLVQPEGSLKLSCATSGFNFNTNAMN |
| | | WVRQAPGKGLEWVARVRNKTNNYATYYADSVKDRFTISRDDSQRMVFLQMNNLKTEDTAI |
| | | YYCVAGNSFAYWGQGTLVTVSPEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPYKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 65 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGGCAGCGGCGGCGGCCTGGTGCAGCCCGAGGGCAGCCTGAAG |
| | | CTGAGCTGCGCCGCCAGCGGCTTCACCTTCAAGACCAACGCCATGAACTGGGTGAGGCAG |
| | | GCCCCCGGCAAGGGCCTGGAGTGGGTGGCCAGGATCAGGAACAAGACCAACAACTACGC |
| | | CACCTACTACGCCGACAGCGTGAAGGACAGGTTCACCATCAGCAGGGACGACAGCCAGA |
| | | GCATGCTGTACCTGCAGATGAACAACCTGAAGACCGAGGACACCGCCATGTACTTCTGCG |
| | | TGGCCGGCAACAGCTTCGCCTACTGGGGCCAGGGCACCCTGGTGACCGTGAGCGCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAAGGTGACCGTGAACTGC |
| | | AAGAGCAGCCAGAGCCTGCTGTACAGCAACAACCAGAAGAACTACCTGGCCTGGTACCA |
| | | GCAGAAGCCCGGCCAGAGCCCCAAGCTGCTGATCTACTGGGCCAGCACCAGGGAGTACG |
| | | GCGTGCCCGACAGGTTCACCGGCAGCGGCAGCGGCACCGACTTCACCCTGACCATCAGCA |
| | | GCGTGAAGGCCGAGGACCTGGCCGTGTACTACTGCCAGCAGTACTACAACTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTACCAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 66 | Amino Acid | MALPVTALLLPLALLLHAARPEVQLVGSGGGLVQPEGSLKLSCAASGFTFKTNAMNWVRQA |
| | | PGKGLEWVARIRNKTNNYATYYADSVKDRFTISRDDSQSMLYLQMNNLKTEDTAMYFCVAG |
| | | NSFAYWGQGTLVTVSAGSTSGSGKPGSGEGSTKGDIVMSQSPSSLAVSVGEKVTVNCKSSQS |
| | | LLYSNNQKNYLAWYQQKPGQSPKLLIYWASTREYGVPDRFTGSGSGTDFTLTISSVKAEDLA |
| | | VYYCQQYYNYPLTFGAGTKLELKEQKLISEEDLNPGGGGGSGGGGSGGGGSGGGGSGSMDI |
| | | QMTQSPSSLSASVGDRVTITCRASQDIRNYLNWYQQKPGKAPKLLIYYTSRLESGVPSRFSGS |
| | | GSGTDYTLTISSLQPEDFATYYCQQGNTLPWTFGQGTKVEIKGGGGSGGGGSGGGGSEVQLV |
| | | ESGGGLVQPGGSLRLSCAASGYSFTGYTMNWVRQAPGKGLEWVALINPTKGVSTYNQKFKD |
| | | RFTISVDKSKNTAYLQMNSLRAEDTAVYYCARSGYYGDSDWYFDVWGQGTLVTVSSTSGGG |
| | | GSLESGQVLLESNIKVLPTWSTPVQPMALIVLGGVAGLLLFIGLGIFFCVRCRHRRRQAERMSQ |
| | | IKRLLSEKKTCQCPHRFQKTCSPI |
| 67 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAAGGTG |
| | | ACCGTGAACTGCAAGAGCAGCCAGAGCCTGCTGTACAGCAACAACCAGAAGAACTACCT |
| | | GGCCTGGTACCAGCAGAAGCCCGGCCAGAGCCCCAAGCTGCTGATCTACTGGGCCAGCA |
| | | CCAGGGAGTACGGCGTGCCCGACAGGTTCACCGGCAGCGGCAGCGGCACCGACTTCACC |
| | | CTGACCATCAGCAGCGTGAAGGCCGAGGACCTGGCCGTGTACTACTGCCAGCAGTACTAC |
| | | AACTACCCCCTGACCTTCGGCGCCGGCACCAAGCTGGAGCTGAAGGGGTCAACCTCTGGT |
| | | AGCGGTAAGCCTGGCTCCGGCGAAGGCTCCACAAAGGGTGAGGTGCAGCTGGTGGGCAG |
| | | CGGCGGCGGCCTGGTGCAGCCCGAGGGCAGCCTGAAGCTGAGCTGCGCCGCCAGCGGCT |
| | | TCACCTTCAAGACCAACGCCATGAACTGGGTGAGGCAGGCCCCCGGCAAGGGCCTGGAG |
| | | TGGGTGGCCAGGATCAGGAACAAGACCAACAACTACGCCACCTACTACGCCGACAGCGT |
| | | GAAGGACAGGTTCACCATCAGCAGGGACGACAGCCAGAGCATGCTGTACCTGCAGATGA |
| | | ACAACCTGAAGACCGAGGACACCGCCATGTACTTCTGCGTGGCCGGCAACAGCTTCGCCT |
| | | ACTGGGGCCAGGGCACCCTGGTGACCGTGAGCGCCGAGCAGAAACTGATCAGCGAGGAA |
| | | GATCTGAATCCTGGCGGAGGCGGAGGAAGTGGTGGCGGAGGTTCTGGTGGCGGTGGATC |
| | | AGGCGGTGGCGGATCTGGATCTATGGATATCCAGATGACCCAGTCCCCGAGCTCCCTGTC |
| | | CGCCTCTGTGGGCGATAGGGTCACCATCACCTGCCGTGCCAGTCAGGACATCCGTAATTA |
| | | TCTGAACTGGTATCAACAGAAACCAGGAAAAGCTCCGAAACTACTGATTTACTATACCTC |
| | | CCGCCTGGAGTCTGGAGTCCCTTCTCGCTTCTCTGGTTCTGGTTCTGGGACGGATTACACT |
| | | CTGACCATCAGCAGTCTGCAACCGGAAGACTTCGCAACTTATTACTGTCAGCAAGGTAAT |
| | | ACTCTGCCGTGGACGTTCGGACAGGGCACCAAGGTGGAGATCAAAGGCGGCGGCGGAAG |
| | | TGGAGGAGGAGGCTCAGGCGGAGGAGGGAGCGAGGTTCAGCTGGTGGAGTCTGGCGGTG |
| | | GCCTGGTGCAGCCAGGGGGCTCACTCCGTTTGTCCTGTGCAGCTTCTGGCTACTCCTTTAC |
| | | CGGCTACACTATGAACTGGGTGCGTCAGGCCCCAGGTAAGGGCCTGGAATGGGTTGCACT |
| | | GATTAATCCTACCAAAGGTGTTAGTACCTACAACCAGAAGTTCAAGGACCGTTTCACTAT |
| | | AAGCGTAGATAAATCCAAAAACACAGCCTACCTGCAAATGAACAGCCTGCGTGCTGAGG |
| | | ACACTGCCGTCTATTATTGTGCTAGAAGCGGATACTACGGCGATAGTGACTGGTATTTTGA |
| | | CGTGTGGGGTCAAGGAACCCTGGTCACCGTCTCCTCGACATCTGGCGGCGGAGGATCTCT |
| | | GGAATCTGGACAGGTGCTGCTGGAAAGCAACATCAAGGTGCTGCCCACCTGGTCTACCCC |
| | | AGTTCAGCCTATGGCTCTGATTGTGCTTGGCGGAGTTGCCGGCCTGCTGCTCTTTATCGGC |
| | | CTGGGCATCTTCTTTTGCGTGCGGTGCAGACATCGGCGGAGACAGGCTGAGAGAATGAGC |
| | | CAGATCAAGCGGCTGCTGAGCGAGAAGAAAACCTGTCAGTGCCCTCACCGGTTCCAGAA |
| | | AACATGCAGCCCCATC |
| 68 | Amino Acid | MALPVTALLLPLALLLHAARPDIVMSQSPSSLAVSVGEKVTVNCKSSQSLLYSNNQKNYLAW |
| | | YQQKPGQSPKLLIYWASTREYGVPDRFTGSGSGTDFTLTISSVKAEDLAVYYCQQYYNYPLTF |
| | | GAGTKLELKGSTSGSGKPGSGEGSTKGEVQLVGSGGGLVQPEGSLKLSCAASGFTFKTNAMN |
| | | WVRQAPGKGLEWVARIRNKTNNYATYYADSVKDRFTISRDDSQSMLYLQMNNLKTEDTAM |
| | | YFCVAGNSFAYWGQGTLVTVSAEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPTKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 69 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGAGACCGGCGGCGGCCTGGTGCAGCCCAAGGGCAGCCTGAAG |
| | | CTGAGCTGCGCCGCCAGCGGCTTCACCTTCAACACCAACGCCATGAACTGGGTGAGGCAG |
| | | GCCCCCGGCAAGGGCCTGCAGTGGGTGGCCAGGGTGAGGAACAAGACCAACAACTACGC |
| | | CACCTACTACGCCGACAGCGTGAAGGACAGGTTCACCATCAGCAGGGACGACAGCCAGA |
| | | GCATGCTGTACCTGCAGATGAACAACCTGAAGACCGAGGACACCGCCATCTACTACTGCG |
| | | TGGGCGGCAACAGCTTCGCCTACTGGGGCCAGGGCACCCTGGTGACCGTGAGCGCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAAGGTGACCATGAGCTGC |
| | | AAGAGCAGCCAGAGCCTGCTGTACAGCAACAACCAGAAGAACTACCTGGCCTGGTACCA |
| | | CCAGAAGCCCGGCCAGAGCCCCAAGCTGCTGATCTACTGGGCCAGCACCAGGGAGAGCG |
| | | GCGTGCCCGACAGGTTCACCGGCAGCGGCAGCGGCACCGACTTCACCCTGACCATCAGCA |
| | | GCGTGAAGGCCGAGGACCTGGCCGTGTACTACTGCCAGCAGTACTACAGCTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTACCAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 70 | Amino Acid | MALPVTALLLPLALLLHAARPEVQLVETGGGLVQPKGSLKLSCAASGFTFNTNAMNWVRQA |
| | | PGKGLQWVARVRNKTNNYATYYADSVKDRFTISRDDSQSMLYLQMNNLKTEDTAIYYCVG |
| | | GNSFAYWGQGTLVTVSAGSTSGSGKPGSGEGSTKGDIVMSQSPSSLAVSVGEKVTMSCKSSQ |
| | | SLLYSNNQKNYLAWYHQKPGQSPKLLIYWASTRESGVPDRFTGSGSGTDFTLTISSVKAEDLA |
| | | VYYCQQYYSYPLTFGAGTKLELKEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPTKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 71 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAAGGTG |
| | | ACCATGAGCTGCAAGAGCAGCCAGAGCCTGCTGTACAGCAACAACCAGAAGAACTACCT |
| | | GGCCTGGTACCACCAGAAGCCCGGCCAGAGCCCCAAGCTGCTGATCTACTGGGCCAGCAC |
| | | CAGGGAGAGCGGCGTGCCCGACAGGTTCACCGGCAGCGGCAGCGGCACCGACTTCACCC |
| | | TGACCATCAGCAGCGTGAAGGCCGAGGACCTGGCCGTGTACTACTGCCAGCAGTACTACA |
| | | GCTACCCCCTGACCTTCGGCGCCGGCACCAAGCTGGAGCTGAAGGGGTCAACCTCTGGTA |
| | | GCGGTAAGCCTGGCTCCGGCGAAGGCTCCACAAAGGGTGAGGTGCAGCTGGTGGAGACC |
| | | GGCGGCGGCCTGGTGCAGCCCAAGGGCAGCCTGAAGCTGAGCTGCGCCGCCAGCGGCTT |
| | | CACCTTCAACACCAACGCCATGAACTGGGTGAGGCAGGCCCCCGGCAAGGGCCTGCAGT |
| | | GGGTGGCCAGGGTGAGGAACAAGACCAACAACTACGCCACCTACTACGCCGACAGCGTG |
| | | AAGGACAGGTTCACCATCAGCAGGGACGACAGCCAGAGCATGCTGTACCTGCAGATGAA |
| | | CAACCTGAAGACCGAGGACACCGCCATCTACTACTGCGTGGGCGGCAACAGCTTCGCCTA |
| | | CTGGGGCCAGGGCACCCTGGTGACCGTGAGCGCCGAGCAGAAACTGATCAGCGAGGAAG |
| | | ATCTGAATCCTGGCGGAGGCGGAGGAAGTGGTGGCGGAGGTTCTGGTGGCGGTGGATCA |
| | | GGCGGTGGCGGATCTGGATCTATGGATATCCAGATGACCCAGTCCCCGAGCTCCCTGTCC |
| | | GCCTCTGTGGGCGATAGGGTCACCATCACCTGCCGTGCCAGTCAGGACATCCGTAATTAT |
| | | CTGAACTGGTATCAACAGAAACCAGGAAAAGCTCCGAAACTACTGATTTACTATACCTCC |
| | | CGCCTGGAGTCTGGAGTCCCTTCTCGCTTCTCTGGTTCTGGTTCTGGGACGGATTACACTC |
| | | TGACCATCAGCAGTCTGCAACCGGAAGACTTCGCAACTTATTACTGTCAGCAAGGTAATA |
| | | CTCTGCCGTGGACGTTCGGACAGGGCACCAAGGTGGAGATCAAAGGCGGCGGCGGAAGT |
| | | GGAGGAGGAGGCTCAGGCGGAGGAGGGAGCGAGGTTCAGCTGGTGGAGTCTGGCGGTGG |
| | | CCTGGTGCAGCCAGGGGGCTCACTCCGTTTGTCCTGTGCAGCTTCTGGCTACTCCTTTACC |
| | | GGCTACACTATGAACTGGGTGCGTCAGGCCCCAGGTAAGGGCCTGGAATGGGTTGCACTG |
| | | ATTAATCCTACCAAAGGTGTTAGTACCTACAACCAGAAGTTCAAGGACCGTTTCACTATA |
| | | AGCGTAGATAAATCCAAAAACACAGCCTACCTGCAAATGAACAGCCTGCGTGCTGAGGA |
| | | CACTGCCGTCTATTATTGTGCTAGAAGCGGATACTACGGCGATAGTGACTGGTATTTTGAC |
| | | GTGTGGGGTCAAGGAACCCTGGTCACCGTCTCCTCGACATCTGGCGGCGGAGGATCTCTG |
| | | GAATCTGGACAGGTGCTGCTGGAAAGCAACATCAAGGTGCTGCCCACCTGGTCTACCCCA |
| | | GTTCAGCCTATGGCTCTGATTGTGCTTGGCGGAGTTGCCGGCCTGCTGCTCTTTATCGGCC |
| | | TGGGCATCTTCTTTTGCGTGCGGTGCAGACATCGGCGGAGACAGGCTGAGAGAATGAGCC |
| | | AGATCAAGCGGCTGCTGAGCGAGAAGAAAACCTGTCAGTGCCCTCACCGGTTCCAGAAA |
| | | ACATGCAGCCCCATC |
| 72 | Amino Acid | MALPVTALLLPLALLLHAARPDIVMSQSPSSLAVSVGEKVTMSCKSSQSLLYSNNQKNYLAW |
| | | YHQKPGQSPKLLIYWASTRESGVPDRFTGSGSGTDFTLTISSVKAEDLAVYYCQQYYSYPLTF |
| | | GAGTKLELKGSTSGSGKPGSGEGSTKGEVQLVETGGGLVQPKGSLKLSCAASGFTFNTNAMN |
| | | WVRQAPGKGLQWVARVRNKTNNYATYYADSVKDRFTISRDDSQSMLYLQMNNLKTEDTAI |
| | | YYCVGGNSFAYWGQGTLVTVSAEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPTKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 73 | Nucleotide | ATGGCTCTGCCTGTGACAGCTCTGTTGCTGCCTCTGGCTCTGCTGCTGCATGCTGCTAGAC |
| | | CTGAGGTGCAGCTGGTGGAGACCGGCGGCGGCCTGGTGCAGCCCGAGGGCAGCCTGAAG |
| | | CTGAGCTGCGCCACCAGCGGCTTCAACTTCAACACCAACGCCATGAACTGGGTGAGGCAG |
| | | GCCCCCGGCAAGGGCCTGGAGTGGGTGGCCAGGGTGAGGAACAAGACCAACAACTACGC |
| | | CACCTACTACGCCGACAGCGTGAAGGACAGGTTCACCATCAGCAGGGACGACAGCCAGA |
| | | GGATGGTGTTCCTGCAGATGAACAACCTGAAGACCGAGGACACCGCCATCTACTACTGCG |
| | | TGGCCGGCAACAGCTTCGCCTACTGGGGCCAGGGCACCCTGGTGACCGTGAGCCCCGGGT |
| | | CAACCTCTGGTAGCGGTAAGCCTGGCTCCGGCGAAGGCTCCACAAAGGGTGACATCGTGA |
| | | TGAGCCAGAGCCCCAGCAGCCTGGCCGTGAGCGTGGGCGAGAGGGTGACCATGAACTGC |
| | | AAGAGCAGCCAGAGCCTGCTGTACAGCAGCAACCAGAAGAACTACCTGGCCTGGTACCA |
| | | GCAGAAGCCCGGCCAGAGCCCCAAGCTGCTGATCTACTGGGCCAGCACCAGGGAGAGCG |
| | | GCGTGCCCGACAGGTTCACCGGCAGCGTGAGCGGCACCGACTTCACCCTGACCATCAGCA |
| | | GCGTGAAGGCCGAGGACCTGGCCGTGTACTACTGCCAGCAGTACTACAACTACCCCCTGA |
| | | CCTTCGGCGCCGGCACCAAGCTGGAGCTGAAGGAGCAGAAACTGATCAGCGAGGAAGAT |
| | | CTGAATCCTGGCGGAGGCGGAGGAAGTGGTGGCGGAGGTTCTGGTGGCGGTGGATCAGG |
| | | CGGTGGCGGATCTGGATCTATGGATATCCAGATGACCCAGTCCCCGAGCTCCCTGTCCGC |
| | | CTCTGTGGGCGATAGGGTCACCATCACCTGCCGTGCCAGTCAGGACATCCGTAATTATCT |
| | | GAACTGGTATCAACAGAAACCAGGAAAAGCTCCGAAACTACTGATTTACTATACCTCCCG |
| | | CCTGGAGTCTGGAGTCCCTTCTCGCTTCTCTGGTTCTGGTTCTGGGACGGATTACACTCTG |
| | | ACCATCAGCAGTCTGCAACCGGAAGACTTCGCAACTTATTACTGTCAGCAAGGTAATACT |
| | | CTGCCGTGGACGTTCGGACAGGGCACCAAGGTGGAGATCAAAGGCGGCGGCGGAAGTGG |
| | | AGGAGGAGGCTCAGGCGGAGGAGGGAGCGAGGTTCAGCTGGTGGAGTCTGGCGGTGGCC |
| | | TGGTGCAGCCAGGGGGCTCACTCCGTTTGTCCTGTGCAGCTTCTGGCTACTCCTTTACCGG |
| | | CTACACTATGAACTGGGTGCGTCAGGCCCCAGGTAAGGGCCTGGAATGGGTTGCACTGAT |
| | | TAATCCTACCAAAGGTGTTAGTACCTACAACCAGAAGTTCAAGGACCGTTTCACTATAAG |
| | | CGTAGATAAATCCAAAAACACAGCCTACCTGCAAATGAACAGCCTGCGTGCTGAGGACA |
| | | CTGCCGTCTATTATTGTGCTAGAAGCGGATACTACGGCGATAGTGACTGGTATTTTGACGT |
| | | GTGGGGTCAAGGAACCCTGGTCACCGTCTCCTCGACATCTGGCGGCGGAGGATCTCTGGA |
| | | ATCTGGACAGGTGCTGCTGGAAAGCAACATCAAGGTGCTGCCCACCTGGTCTACCCCAGT |
| | | TCAGCCTATGGCTCTGATTGTGCTTGGCGGAGTTGCCGGCCTGCTGCTCTTTATCGGCCTG |
| | | GGCATCTTCTTTTGCGTGCGGTGCAGACATCGGCGGAGACAGGCTGAGAGAATGAGCCAG |
| | | ATCAAGCGGCTGCTGAGCGAGAAGAAAACCTGTCAGTGCCCTCACCGGTTCCAGAAAAC |
| | | ATGCAGCCCCATC |
| 74 | Amino Acid | MALPVTALLLPLALLLHAARPEVQLVETGGGLVQPEGSLKLSCATSGFNFNTNAMNWVRQA |
| | | PGKGLEWVARVRNKTNNYATYYADSVKDRFTISRDDSQRMVFLQMNNLKTEDTAIYYCVA |
| | | GNSFAYWGQGTLVTVSPGSTSGSGKPGSGEGSTKGDIVMSQSPSSLAVSVGERVTMNCKSSQ |
| | | SLLYSSNQKNYLAWYQQKPGQSPKLLIYWASTRESGVPDRFTGSVSGTDFTLTISSVKAEDLA |
| | | VYYCQQYYNYPLTFGAGTKLELKEQKLISEEDLNPGGGGGSGGGGSGGGGSGGGGSGSMDI |
| | | QMTQSPSSLSASVGDRVTITCRASQDIRNYLNWYQQKPGKAPKLLIYYTSRLESGVPSRFSGS |
| | | GSGTDYTLTISSLQPEDFATYYCQQGNTLPWTFGQGTKVEIKGGGGSGGGGSGGGGSEVQLV |
| | | ESGGGLVQPGGSLRLSCAASGYSFTGYTMNWVRQAPGKGLEWVALINPTKGVSTYNQKFKD |
| | | RFTISVDKSKNTAYLQMNSLRAEDTAVYYCARSGYYGDSDWYFDVWGQGTLVTVSSTSGGG |
| | | GSLESGQVLLESNIKVLPTWSTPVQPMALIVLGGVAGLLLFIGLGIFFCVRCRHRRRQAERMSQ |
| | | IKRLLSEKKTCQCPHRFQKTCSPI |
| 75 | Nucleotide | ATGGCTCTGCCTGTGACAGCTCTGTTGCTGCCTCTGGCTCTGCTGCTGCATGCTGCTAGAC |
| | | CTGACATCGTGATGAGCCAGAGCCCCAGCAGCCTGGCCGTGAGCGTGGGCGAGAGGGTG |
| | | ACCATGAACTGCAAGAGCAGCCAGAGCCTGCTGTACAGCAGCAACCAGAAGAACTACCT |
| | | GGCCTGGTACCAGCAGAAGCCCGGCCAGAGCCCCAAGCTGCTGATCTACTGGGCCAGCA |
| | | CCAGGGAGAGCGGCGTGCCCGACAGGTTCACCGGCAGCGTGAGCGGCACCGACTTCACC |
| | | CTGACCATCAGCAGCGTGAAGGCCGAGGACCTGGCCGTGTACTACTGCCAGCAGTACTAC |
| | | AACTACCCCCTGACCTTCGGCGCCGGCACCAAGCTGGAGCTGAAGGGGTCAACCTCTGGT |
| | | AGCGGTAAGCCTGGCTCCGGCGAAGGCTCCACAAAGGGTGAGGTGCAGCTGGTGGAGAC |
| | | CGGCGGCGGCCTGGTGCAGCCCGAGGGCAGCCTGAAGCTGAGCTGCGCCACCAGCGGCT |
| | | TCAACTTCAACACCAACGCCATGAACTGGGTGAGGCAGGCCCCCGGCAAGGGCCTGGAG |
| | | TGGGTGGCCAGGGTGAGGAACAAGACCAACAACTACGCCACCTACTACGCCGACAGCGT |
| | | GAAGGACAGGTTCACCATCAGCAGGGACGACAGCCAGAGGATGGTGTTCCTGCAGATGA |
| | | ACAACCTGAAGACCGAGGACACCGCCATCTACTACTGCGTGGCCGGCAACAGCTTCGCCT |
| | | ACTGGGGCCAGGGCACCCTGGTGACCGTGAGCCCCGAGCAGAAACTGATCAGCGAGGAA |
| | | GATCTGAATCCTGGCGGAGGCGGAGGAAGTGGTGGCGGAGGTTCTGGTGGCGGTGGATC |
| | | AGGCGGTGGCGGATCTGGATCTATGGATATCCAGATGACCCAGTCCCCGAGCTCCCTGTC |
| | | CGCCTCTGTGGGCGATAGGGTCACCATCACCTGCCGTGCCAGTCAGGACATCCGTAATTA |
| | | TCTGAACTGGTATCAACAGAAACCAGGAAAAGCTCCGAAACTACTGATTTACTATACCTC |
| | | CCGCCTGGAGTCTGGAGTCCCTTCTCGCTTCTCTGGTTCTGGTTCTGGGACGGATTACACT |
| | | CTGACCATCAGCAGTCTGCAACCGGAAGACTTCGCAACTTATTACTGTCAGCAAGGTAAT |
| | | ACTCTGCCGTGGACGTTCGGACAGGGCACCAAGGTGGAGATCAAAGGCGGCGGCGGAAG |
| | | TGGAGGAGGAGGCTCAGGCGGAGGAGGGAGCGAGGTTCAGCTGGTGGAGTCTGGCGGTG |
| | | GCCTGGTGCAGCCAGGGGGCTCACTCCGTTTGTCCTGTGCAGCTTCTGGCTACTCCTTTAC |
| | | CGGCTACACTATGAACTGGGTGCGTCAGGCCCCAGGTAAGGGCCTGGAATGGGTTGCACT |
| | | GATTAATCCTACCAAAGGTGTTAGTACCTACAACCAGAAGTTCAAGGACCGTTTCACTAT |
| | | AAGCGTAGATAAATCCAAAAACACAGCCTACCTGCAAATGAACAGCCTGCGTGCTGAGG |
| | | ACACTGCCGTCTATTATTGTGCTAGAAGCGGATACTACGGCGATAGTGACTGGTATTTTGA |
| | | CGTGTGGGGTCAAGGAACCCTGGTCACCGTCTCCTCGACATCTGGCGGCGGAGGATCTCT |
| | | GGAATCTGGACAGGTGCTGCTGGAAAGCAACATCAAGGTGCTGCCCACCTGGTCTACCCC |
| | | AGTTCAGCCTATGGCTCTGATTGTGCTTGGCGGAGTTGCCGGCCTGCTGCTCTTTATCGGC |
| | | CTGGGCATCTTCTTTTGCGTGCGGTGCAGACATCGGCGGAGACAGGCTGAGAGAATGAGC |
| | | CAGATCAAGCGGCTGCTGAGCGAGAAGAAAACCTGTCAGTGCCCTCACCGGTTCCAGAA |
| | | AACATGCAGCCCCATC |
| 76 | Amino Acid | MALPVTALLLPLALLLHAARPDIVMSQSPSSLAVSVGERVTMNCKSSQSLLYSSNQKNYLAW |
| | | YQQKPGQSPKLLIYWASTRESGVPDRFTGSVSGTDFTLTISSVKAEDLAVYYCQQYYNYPLTF |
| | | GAGTKLELKGSTSGSGKPGSGEGSTKGEVQLVETGGGLVQPEGSLKLSCATSGFNFNTNAMN |
| | | WVRQAPGKGLEWVARVRNKTNNYATYYADSVKDRFTISRDDSQRMVFLQMNNLKTEDTAI |
| | | YYCVAGNSFAYWGQGTLVTVSPEQKLISEEDLNPGGGGGSGGGGSGGGGSGGGGSGSMDIQ |
| | | MTQSPSSLSASVGDRVTITCRASQDIRNYLNWYQQKPGKAPKLLIYYTSRLESGVPSRFSGSGS |
| | | GTDYTLTISSLQPEDFATYYCQQGNTLPWTFGQGTKVEIKGGGGSGGGGSGGGGSEVQLVES |
| | | GGGLVQPGGSLRLSCAASGYSFTGYTMNWVRQAPGKGLEWVALINPTKGVSTYNQKFKDRF |
| | | TISVDKSKNTAYLQMNSLRAEDTAVYYCARSGYYGDSDWYFDVWGQGTLVTVSSTSGGGGS |
| | | LESGQVLLESNIKVLPTWSTPVQPMALIVLGGVAGLLLFIGLGIFFCVRCRHRRRQAERMSQIK |
| | | RLLSEKKTCQCPHRFQKTCSPI |
| 77 | Amino Acid | TNAMN |
| 78 | Amino Acid | RIRNKTNNYATYYADSVKD |
| 79 | Amino Acid | GNSFAY |
| 80 | Amino Acid | KSSQSLLYSNNQKNYLA |
| 81 | Amino Acid | WASTREY |
| 82 | Amino Acid | QQYYNYPLT |
| 83 | Amino Acid | GFTFKTNA |
| 84 | Amino Acid | IRNKTNNYAT |
| 85 | Amino Acid | VAGNSFAY |
| 86 | Amino Acid | QSLLYSNNQKNY |
| 87 | Amino Acid | WAS |
| 88 | Amino Acid | RVRNKTNNYATYYADSVKD |
| 89 | Amino Acid | WASTRES |
| 90 | Amino Acid | QQYYSYPLT |
| 91 | Amino Acid | GFTFNTNA |
| 92 | Amino Acid | VRNKTNNYAT |
| 93 | Amino Acid | VGGNSFAY |
| 94 | Amino Acid | KSSQSLLYSSNQKNYLA |
| 95 | Amino Acid | GFNFNTNA |
| 96 | Amino Acid | QSLLYSSNQKNY |
| 97 | Amino Acid | EVQLVGSGGGLVQPEGSLKLSCAASGFTFKTNAMNWVRQAPGKGLEWVARIRNKTNNYAT |
| | | YYADSVKDRFTISRDDSQSMLYLQMNNLKTEDTAMYFCVAGNSFAYWGQGTLVTVSAGSTS |
| | | GSGKPGSGEGSTKGDIVMSQSPSSLAVSVGEKVTVNCKSSQSLLYSNNQKNYLAWYQQKPG |
| | | QSPKLLIYWASTREYGVPDRFTGSGSGTDFTLTISSVKAEDLAVYYCQQYYNYPLTFGAGTKL |
| | | ELK |
| 98 | Amino Acid | DIVMSQSPSSLAVSVGEKVTVNCKSSQSLLYSNNQKNYLAWYQQKPGQSPKLLIYWASTREY |
| | | GVPDRFTGSGSGTDFTLTISSVKAEDLAVYYCQQYYNYPLTFGAGTKLELKGSTSGSGKPGSG |
| | | EGSTKGEVQLVGSGGGLVQPEGSLKLSCAASGFTFKTNAMNWVRQAPGKGLEWVARIRNKT |
| | | NNYATYYADSVKDRFTISRDDSQSMLYLQMNNLKTEDTAMYFCVAGNSFAYWGQGTLVTV |
| | | SA |
| 99 | Amino Acid | EVQLVETGGGLVQPKGSLKLSCAASGFTFNTNAMNWVRQAPGKGLQWVARVRNKTNNYAT |
| | | YYADSVKDRFTISRDDSQSMLYLQMNNLKTEDTAIYYCVGGNSFAYWGQGTLVTVSAGSTS |
| | | GSGKPGSGEGSTKGDIVMSQSPSSLAVSVGEKVTMSCKSSQSLLYSNNQKNYLAWYHQKPG |
| | | QSPKLLIYWASTRESGVPDRFTGSGSGTDFTLTISSVKAEDLAVYYCQQYYSYPLTFGAGTKL |
| | | ELK |
| 100 | Amino Acid | DIVMSQSPSSLAVSVGEKVTMSCKSSQSLLYSNNQKNYLAWYHQKPGQSPKLLIYWASTRES |
| | | GVPDRFTGSGSGTDFTLTISSVKAEDLAVYYCQQYYSYPLTFGAGTKLELKGSTSGSGKPGSG |
| | | EGSTKGEVQLVETGGGLVQPKGSLKLSCAASGFTFNTNAMNWVRQAPGKGLQWVARVRNK |
| | | TNNYATYYADSVKDRFTISRDDSQSMLYLQMNNLKTEDTAIYYCVGGNSFAYWGQGTLVTV |
| | | SA |
| 101 | Amino Acid | EVQLVETGGGLVQPEGSLKLSCATSGFNFNTNAMNWVRQAPGKGLEWVARVRNKTNNYAT |
| | | YYADSVKDRFTISRDDSQRMVFLQMNNLKTEDTAIYYCVAGNSFAYWGQGTLVTVSPGSTS |
| | | GSGKPGSGEGSTKGDIVMSQSPSSLAVSVGERVTMNCKSSQSLLYSSNQKNYLAWYQQKPGQ |
| | | SPKLLIYWASTRESGVPDRFTGSVSGTDFTLTISSVKAEDLAVYYCQQYYNYPLTFGAGTKLE |
| | | LK |
| 102 | Amino Acid | DIVMSQSPSSLAVSVGERVTMNCKSSQSLLYSSNQKNYLAWYQQKPGQSPKLLIYWASTRES |
| | | GVPDRFTGSVSGTDFTLTISSVKAEDLAVYYCQQYYNYPLTFGAGTKLELKGSTSGSGKPGSG |
| | | EGSTKGEVQLVETGGGLVQPEGSLKLSCATSGFNFNTNAMNWVRQAPGKGLEWVARVRNK |
| | | TNNYATYYADSVKDRFTISRDDSQRMVFLQMNNLKTEDTAIYYCVAGNSFAYWGQGTLVTV |
| | | SP |

What is claimed is:

1. A nucleic acid encoding a Glypican 3 (GPC3) T cell-antigen coupler (GPC3-TAC) polypeptide, the nucleic acid comprising:
(a) a first polynucleotide encoding an antigen-binding domain that binds GPC3;
(b) a second polynucleotide encoding an antigen-binding domain that binds a protein associated with a TCR complex; and
(c) a third polynucleotide encoding a TCR co-receptor cytosolic domain and transmembrane domain;
wherein components encoded by (a), components encoded by (b), and components encoded by (c) are fused directly to each other, or joined by at least one linker.

2. The nucleic acid of claim 1, wherein the first polynucleotide, the second polynucleotide, and the third polynucleotide are in order.

3. The nucleic acid of claim 1 or 2, wherein the antigen-binding domain that binds GPC3 is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

4. The nucleic acid of any one of claims 1-3, wherein the antigen-binding domain that binds GPC3 is a designed ankyrin repeat (DARPin) polypeptide, a single chain variable fragment (scFv), or a nanobody.

5. The nucleic acid of any one of claims 1-4, wherein the antigen-binding domain that binds GPC3 is a nanobody.

6. The nucleic acid of any one of claims 1-4, wherein the antigen-binding domain that binds GPC3 comprises an antigen-binding domain derived from an antibody selected from codrituzumab/GC33, GPC3-C02, A1836A, 1G12, YP8, YP9, or YP9.1.

7. The nucleic acid of any one of claim 1-4 or 6, wherein the antigen-binding domain that binds GPC3 comprises an amino acid sequence having at least 80%, at least 85%, at least 90%, at least 95%, at least 96%, at least 97%, at least 98%, at least 99%, or 100% sequence identity with the amino acid sequence of the antigen-binding domain(s) of codrituzumab/GC33, GPC3-C02, A1836A 1G12, YP8, YP9, or YP9.1.

8. The nucleic acid of any one of claim 1-4, 6, or 7, wherein the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1.

9. The nucleic acid of any one of claim 1-4 or 6-8, wherein the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1, and the non-CDR sequences of the antigen-binding domain that binds GPC3 have at least 80%, at least 85%, at least 90%, at least 95%, at least 96%, at least 97%, at least 98%, or at least 99% sequence identity with the non-CDR sequences of the amino acid sequence of codrituzumab/GC33, GPC3-C02, A1836A, 1G12, YP8, YP9, or YP9.1.

10. The nucleic acid sequence of any one of claim 1-4 or 6-9, wherein the antigen-binding domain comprises:
(a) a heavy chain complementarity-determining region 1 (HCDR1) of SEQ ID NO: 77, a heavy chain complementarity-determining region 2 (HCDR2) of SEQ ID NO: 78, and a heavy chain complementarity-determining region 3 (HCDR3) of SEQ ID NO: 79; and a light-chain variable domain comprising a light chain complementarity-determining region 1 (LCDR1) of SEQ ID NO: 80, a light chain complementarity-determining region 2 (LCDR2) of SEQ ID NO: 81, and a light chain complementarity-determining region 3 (LCDR3) of SEQ ID NO: 82;
(b) an HCDR1 of SEQ ID NO: 83, an HCDR2 of SEQ ID NO: 84, and an HCDR3 of SEQ ID NO: 85; and a light-chain variable domain comprising an LCDR1 of SEQ ID NO: 86, an LCDR2 of SEQ ID NO: 87, and an LCDR3 of SEQ ID NO: 82;
(c) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 80, an LCDR2 of SEQ ID NO: 89, and an LCDR3 of SEQ ID NO: 90;
(d) an HCDR1 of SEQ ID NO: 91, an HCDR2 of SEQ ID NO: 92, and an HCDR3 of SEQ ID NO: 93; and a light-chain variable domain comprising an LCDR1 of SEQ ID NO: 86, an LCDR2 of SEQ ID NO: 87, and an LCDR3 of SEQ ID NO: 90;
(e) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 94, an LCDR2 of SEQ ID NO: 89, and an LCDR3 of SEQ ID NO: 82; or
(f) an HCDR1 of SEQ ID NO: 95, an HCDR2 of SEQ ID NO: 92, and an HCDR3 of SEQ ID NO: 85; and a light-chain variable domain comprising an LCDR1 of SEQ ID NO: 96, an LCDR2 of SEQ ID NO: 87, and an LCDR3 of SEQ ID NO: 82.

11. The nucleic acid sequence of any one of claim 1-4 or 6-10, wherein the antigen-binding domain comprises an amino acid sequence having at least 80%, at least 85%, at least 90%, at least 95%, at least 96%, at least 97%, at least 98%, at least 99%, or 100% sequence identity with an amino acid sequence selected from SEQ ID NOs: 97-102.

12. The nucleic acid of any one of claims 1-11, wherein the protein associated with the TCR complex is a CD3 protein.

13. The nucleic acid of claim 12, wherein the CD3 protein is a CD3γ protein, CD3δ protein and/or CD3ε protein. In some embodiments, the CD3 protein is a CD3ε protein.

14. The nucleic acid of claim 13, wherein the CD3 protein is a CD3ε protein.

15. The nucleic acid of any one of claims 1-14, wherein the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

16. The nucleic acid of any one of claims 1-15, wherein the antigen-binding domain that binds the protein associated with the TCR complex is derived from an antibody selected from UCHT1 OKT3, F6A, and L2K.

17. The nucleic acid of claim 16, wherein the antigen-binding domain that binds the protein associated with the TCR complex is a UCHT1 antigen-binding domain.

18. The nucleic acid of claim 17, wherein the UCHT1 antigen-binding domain is an scFv of UCHT1.

19. The nucleic acid of claim 17 or 18, wherein the UCHT1 antigen-binding domain comprises a Y to T mutation at a position corresponding to amino acid 182 of SEQ ID NO: 32 (Y182T).

20. The nucleic acid of any one of claims 17-19, wherein the UCHT1 antigen-binding domain comprises a humanized variant of UCHT1 (huUCHT1).

21. The nucleic acid of claim 20, wherein the UCHT1 antigen-binding domain comprises a humanized variant of UCHT1 comprising a Y to T mutation at a position corresponding to amino acid 177 of SEQ ID NO: 40 (huUCHT1 (Y177T)).

22. The nucleic acid of any one of claims 17-21, wherein antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

23. The nucleic acid of any one of claims 17-22, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

24. The nucleic acid of any one of claims 17-23, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

25. The nucleic acid of claim 16, wherein the antigen-binding domain that binds the protein associated with the TCR complex is an OKT3 antigen-binding domain.

26. The nucleic acid of claim 25, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3).

27. The nucleic acid of claim 25 or 26, wherein the CDR sequences of the antigen-binding domain that binds the protein associated with the TCR complex have 100% identity with the CDR sequences of the amino acid sequence of SEQ ID NO: 34 (OKT3).

28. The nucleic acid of any one of claims 25-27, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3).

29. The nucleic acid of claim 16, wherein the antigen-binding domain that binds the protein associated with the TCR complex is a F6A antigen-binding domain.

30. The nucleic acid of claim 29, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A).

31. The nucleic acid of claim 29 or 30, wherein the CDR sequences of the antigen-binding domain that binds the protein associated with the TCR complex have 100% identity with the CDR sequences of the amino acid sequence of SEQ ID NO: 36 (F6A).

32. The nucleic acid of any one of claims 29-31, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A).

33. The nucleic acid of claim 16, wherein the antigen-binding domain that binds the protein associated with the TCR complex is a L2K antigen-binding domain.

34. The nucleic acid of claim 33, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K).

35. The nucleic acid of claim 33 or 34, wherein the CDR sequences of the antigen-binding domain that binds the protein associated with the TCR complex have 100% identity with the CDR sequences of the amino acid sequence of SEQ ID NO: 38 (L2K).

36. The nucleic acid of any one of claims 33-35, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K).

37. The nucleic acid of any one of claims 1-36, wherein the transmembrane domain is a CD4 transmembrane domain and the cytosolic domain is a CD4 cytosolic domain.

38. The nucleic acid of claim 37, wherein the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain).

39. The nucleic acid of any one of claims 1-36, wherein the transmembrane domain is a CD8 transmembrane domain and the cytosolic domain is a CD8 cytosolic domain.

40. The nucleic acid of any one of claims 1-39, wherein the component encoded by (a) and the component encoded by (c) are fused to the component encoded by (b).

41. The nucleic acid of any one of claims 1-39, wherein the component encoded by (b) and the component encoded by (c) are fused to the component encoded by (a).

42. The nucleic acid of any one of claims 1-41, wherein at least one linker joins the component encoded by (a) to the component encoded by (b).

43. The nucleic acid of claim 42, wherein the at least one linker is a glycine and/or serine-rich linker, a large protein domain, a long helix structure, or a short helix structure.

44. The nucleic acid of claim 42 or 43, wherein the at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker).

45. The nucleic acid of any one of claims 1-44, wherein the nucleic acid sequence does not encode a co-stimulatory domain.

46. The nucleic acid of any one of claims 1-45, wherein the nucleic acid sequence does not encode an activation domain.

47. The nucleic acid of any one of claims 1-46, wherein the nucleic acid sequence further encodes a leader sequence.

48. The nucleic acid of claim 47, wherein the leader sequence comprises an amino acid sequence having at least 80%, at least 85%, at least 90%, at least 95%, at least 96%, at least 97%, at least 98%, at least 99%, or 100% sequence identity with the amino acid sequence of SEQ ID NO: 2 (muIgG leader), SEQ ID NO: 18 (huIgG leader), SEQ ID NO: 20 (huCD8a-1 leader) or SEQ ID NO: 30 (huCD8a-2 leader).

49. A nucleic acid encoding a GPC3 TAC polypeptide, the nucleic acid sequence comprising a sequence having at least 80% sequence identity with a nucleic acid sequence selected from any one of SEQ ID NOs: 53, 55, 57, 59, 61, 63, 65, 67, 69, 71, 73, or 75.

50. A nucleic acid encoding a GPC3 TAC polypeptide, the nucleic acid sequence comprising a sequence selected from any one of SEQ ID NOs: 53, 55, 57, 59, 61, 63, 65, 67, 69, 71, 73, or 75.

51. An expression vector comprising the nucleic acid of any one of claims 1-50.

52. The expression vector of claim 51, further comprising a promoter functional in a mammalian cell.

53. The expression vector of claim 51 or 52, wherein the expression vector is a lentiviral vector.

54. The expression vector of claim 53, wherein the lentiviral vector is a VSV-G pseudotyped lentivirus.

55. The expression vector of claim 51 or 52, wherein the expression vector is a γ retroviral vector.

56. The expression vector of claim 55, wherein the γ retroviral vector is a GALV pseudotyped γ-retrovirus.

57. A GPC3-TAC polypeptide, comprising:
(a) an antigen-binding domain that binds GPC3;
(b) an antigen-binding domain that binds a protein associated with a TCR complex; and
(c) a TCR co-receptor cytosolic domain and transmembrane domain;
wherein component (a), component (b), and component (c) are fused directly to each other, or joined by at least one linker.

58. The GPC3-TAC polypeptide of claim 57, wherein the first polynucleotide, the second polynucleotide, and the third polynucleotide are in order.

59. The GPC3-TAC polypeptide of claim 57 or 58, wherein the antigen-binding domain that binds GPC3-TAC is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

60. The GPC3-TAC polypeptide of claim 59, wherein the antigen-binding domain that binds GPC3-TAC is a designed ankyrin repeat (DARPin) polypeptide, a single chain variable fragment (scFv), or a nanobody.

61. The GPC3-TAC polypeptide of claim 59, wherein the antigen-binding domain that binds GPC3 is a nanobody.

62. The GPC3-TAC polypeptide of any one of claims 57-59, wherein the antigen-binding domain that binds GPC3 comprises an antigen-binding domain derived from an antibody selected from codrituzumab/GC33, GPC3-C02, A1836A, 1G12, YP8, YP9, or YP9.1.

63. The GPC3-TAC polypeptide of any one of claim 57-59 or 62, wherein the antigen-binding domain that binds GPC3 comprises an amino acid sequence having at least 80%, at least 85%, at least 90%, at least 95%, at least 96%, at least 97%, at least 98%, at least 99%, or 100% sequence identity with the amino acid sequence of the antigen-binding domain(s) of codrituzumab/GC33, GPC3-C02, A1836A, 1G12, YP8, YP9, or YP9.1.

64. The GPC3-TAC polypeptide of any one of claim 57-59, 62, or 63, wherein the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1.

65. The GPC3-TAC polypeptide of any one of claim 57-59 or 62-64, wherein the CDR sequences of the antigen-binding domain that binds GPC3 have 100% identity with the CDR sequences of the amino acid sequence of codrituzumab/GC33, GPC3-C02, A1836A, 1G12, YP8, YP9, or YP9.1, and the non-CDR sequences of the antigen-binding domain that binds GPC3 have at least 80%, at least 85%, at least 90%, at least 95%, at least 96%, at least 97%, at least 98%, or at least 99% sequence identity with the non-CDR sequences of the amino acid sequence of codrituzumab/GC33, GPC3-C02, A1836A, 1G12, YP8, YP9, or YP9.1.

66. The GPC3-TAC polypeptide of any one of claim 57-59 or 62-65, wherein the antigen-binding domain comprises:
(a) an HCDR1 of SEQ ID NO: 77, an HCDR2 of SEQ ID NO: 78, and an HCDR3 of SEQ ID NO: 79; and a light-chain variable domain comprising an LCDR1 of SEQ ID NO: 80, an LCDR2 of SEQ ID NO: 81, and an LCDR3 of SEQ ID NO: 82;
(b) an HCDR1 of SEQ ID NO: 83, an HCDR2 of SEQ ID NO: 84, and an HCDR3 of SEQ ID NO: 85; and a light-chain variable domain comprising an LCDR1 of SEQ ID NO: 86, an LCDR2 of SEQ ID NO: 87, and an LCDR3 of SEQ ID NO: 82;
(c) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 80, an LCDR2 of SEQ ID NO: 89, and an LCDR3 of SEQ ID NO: 90;
(d) an HCDR1 of SEQ ID NO: 91, an HCDR2 of SEQ ID NO: 92, and an HCDR3 of SEQ ID NO: 93; and a light-chain variable domain comprising an LCDR1 of SEQ ID NO: 86, an LCDR2 of SEQ ID NO: 87, and an LCDR3 of SEQ ID NO: 90;
(e) an HCDR1 of SEQ ID NO: 77, an HCDR2 of SEQ ID NO: 88, and an HCDR3 of SEQ ID NO: 79; and a light-chain variable domain comprising an LCDR1 of SEQ ID NO: 94, an LCDR2 of SEQ ID NO: 89, and an LCDR3 of SEQ ID NO: 82; or
(f) an HCDR1 of SEQ ID NO: 95, an HCDR2 of SEQ ID NO: 92, and an HCDR3 of SEQ ID NO: 85; and a light-chain variable domain comprising an LCDR1 of SEQ ID NO: 96, an LCDR2 of SEQ ID NO: 87, and an LCDR3 of SEQ ID NO: 82.

67. The GPC3-TAC polypeptide of any one of claim 57-59 or 62-66, wherein the antigen-binding domain comprises an amino acid sequence having at least 80%, at least 85%, at least 90%, at least 95%, at least 96%, at least 97%, at least 98%, at least 99%, or 100% sequence identity with an amino acid sequence selected from SEQ ID NOs: 97-102.

68. The GPC3-TAC polypeptide of any one of claims 57-67, wherein the protein associated with the TCR complex is a CD3 protein.

69. The GPC3-TAC polypeptide of claim 68, wherein the CD3 protein is a CD3γ protein, CD3δ protein and/or CD3ε protein. In some embodiments, the CD3 protein is a CD3ε protein.

70. The GPC3-TAC polypeptide of claim 69, wherein the CD3 protein is a CD3ε protein.

71. The GPC3-TAC polypeptide of any one of claims 57-70, wherein the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

72. The GPC3-TAC polypeptide of any one of claims 57-71, wherein the antigen-binding domain that binds the protein associated with the TCR complex is derived from an antibody selected from UCHT1, OKT3, F6A, and L2K.

73. The GPC3-TAC polypeptide of claim 72, wherein the antigen-binding domain that binds the protein associated with the TCR complex is a UCHT1 antigen-binding domain.

74. The GPC3-TAC polypeptide of claim 73, wherein the UCHT1 antigen-binding domain is an scFv of UCHT1.

75. The GPC3-TAC polypeptide of claim 73 or 74, wherein the UCHT1 antigen-binding domain comprises a Y to T mutation at a position corresponding to amino acid 182 of SEQ ID NO: 32 (Y182T).

76. The GPC3-TAC polypeptide of any one of claims 72-75, wherein the UCHT1 antigen-binding domain comprises a humanized variant of UCHT1 (huUCHT1).

77. The GPC3-TAC polypeptide of claim 76, wherein the UCHT1 antigen-binding domain comprises a humanized variant of UCHT1 comprising a Y to T mutation at a position corresponding to amino acid 177 of SEQ ID NO: 40 (huUCHT1 (Y177T)).

78. The GPC3-TAC polypeptide of any one of claims 73-77, wherein antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

79. The GPC3-TAC polypeptide of any one of claims 73-78, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

80. The GPC3-TAC polypeptide of any one of claims 73-79, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

81. The GPC3-TAC polypeptide of claim 72, wherein the antigen-binding domain that binds the protein associated with the TCR complex is an OKT3 antigen-binding domain.

82. The GPC3-TAC polypeptide of claim 81, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3).

83. The GPC3-TAC polypeptide of claim 81 or 82, wherein the CDR sequences of the antigen-binding domain that binds the protein associated with the TCR complex have 100% identity with the CDR sequences of the amino acid sequence of SEQ ID NO: 34 (OKT3).

84. The GPC3-TAC polypeptide of any one of claims 81-83, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3).

85. The GPC3-TAC polypeptide of claim 72, wherein the antigen-binding domain that binds the protein associated with the TCR complex is a F6A antigen-binding domain.

86. The GPC3-TAC polypeptide of claim 85, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A).

87. The GPC3-TAC polypeptide of claim 85 or 86, wherein the CDR sequences of the antigen-binding domain that binds the protein associated with the TCR complex have 100% identity with the CDR sequences of the amino acid sequence of SEQ ID NO: 36 (F6A).

88. The GPC3-TAC polypeptide of any one of claims 85-87, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A).

89. The GPC3-TAC polypeptide of claim 72, wherein the antigen-binding domain that binds the protein associated with the TCR complex is a L2K antigen-binding domain.

90. The GPC3-TAC polypeptide of claim 89, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K).

91. The GPC3-TAC polypeptide of claim 89 or 90, wherein the CDR sequences of the antigen-binding domain that binds the protein associated with the TCR complex have 100% identity with the CDR sequences of the amino acid sequence of SEQ ID NO: 38 (L2K).

92. The GPC3-TAC polypeptide of any one of claims 89-91, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K).

93. The GPC3-TAC polypeptide of any one of claims 57-92, wherein the transmembrane domain is a CD4 transmembrane domain and the cytosolic domain is a CD4 cytosolic domain.

94. The GPC3-TAC polypeptide of claim 93, wherein the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain).

95. The GPC3-TAC polypeptide of claim 57-92, wherein the transmembrane domain is a CD8 transmembrane domain and the cytosolic domain is a CD8 cytosolic domain.

96. The GPC3-TAC polypeptide of any one of claims 57-95, wherein component (a) and component (c) are fused to component (b).

97. The GPC3-TAC polypeptide of any one of claims 57-95, wherein component (b) and component (c) are fused to component (a).

98. The GPC3-TAC polypeptide of any one of claims 57-97, wherein at least one linker joins component (a) to component (b).

99. The GPC3-TAC polypeptide of claim 98, wherein the at least one linker is a glycine and/or serine-rich linker, a large protein domain, a long helix structure, or a short helix structure.

100. The GPC3-TAC polypeptide of claim 99, wherein the at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker).

101. The GPC3-TAC polypeptide of any one of claims 57-100, wherein the GPC3-TAC does not comprise a co-stimulatory domain.

102. The GPC3-TAC polypeptide of any one of claims 57-101, wherein the GPC3-TAC does not comprise an activation domain.

103. A GPC3-TAC polypeptide comprising an amino acid sequence having at least 80% sequence identity with an amino acid sequence selected from any one of SEQ ID NOs: 54, 56, 58, 60, 62, 64, 66, 68, 70, 72, 74, or 76.

104. A GPC3-TAC polypeptide comprising an amino acid sequence selected from any one of SEQ ID NOs: 54, 56, 58, 60, 62, 64, 66, 68, 70, 72, 74, or 76.

105. A T cell comprising the nucleic acid of any one of claims 1-50.

106. A T cell comprising the expression vector of any one of claims 51-56.

107. A T cell comprising the GPC3-TAC polypeptide of any one of claims 57-104.

108. A pharmaceutical composition comprising the T cell of any one of claims 105-107, and a pharmaceutically acceptable excipient.

109. A method of treating a GPC3-expressing cancer in an individual in need thereof, comprising administering to the individual the pharmaceutical composition of claim 108.

110. The method of claim 109, wherein the cancer is a solid cancer.

111. The method of claim 109 or 110, wherein the cancer is a liver cancer (for example, HCC), gastric carcinoma, ovarian carcinoma (for example, ovarian clear cell carcinoma), melanoma, colorectal carcinoma, thyroid cancer, squamous cell carcinoma of the lung, hepatoblastoma, nephroblastoma (for example, Wilms tumor), yolk sac tumor, sarcoma, liposarcoma, pediatric embryonal tumor, rhabdoid tumor (for example, rhabdomyosarcoma), or neuroblastoma.
